(19) United States
(12) Patent Application Publication
(10) Pub. No.: US 2007/0259809 A1
(43) Pub. Date: Nov. 8, 2007

(54) Methods of Using High Affinity Atiii Variants

(76) Inventor: Susan C. Bock, Salt Lake City, UT (US)

Correspondence Address: NEEDLE & ROSENBERG, P.C., SUITE 1000, 999 PEACHTREE STREET, ATLANTA, GA, 30309-3915, US

(21) Appl. No.: 10/584,640

(22) PCT Filed: Jan. 10, 2005
(86) PCT No.: PCT/US05/00843

§ 371 Date: May 18, 2007

Related U.S. Application Data
(60) Provisional application No. 60/535,360, filed on Jan. 9, 2004.
(60) Provisional application No. 60/618,746, filed on Oct. 14, 2004.

(51) Int. Cl. A61K 38/16; A61P 7/02; C07K 14/00; G01N 33/53

(52) U.S. Cl. 514/8; 435/7.8; 530/395

(57) ABSTRACT

Disclosed are compositions and methods related to binding of ATIII under low and high shear rate conditions.

### I. CROSS REFERENCE TO RELATED APPLICATIONS

This application claims priority to U.S. Provisional Application Nos. 60/535,360, filed on Jan. 9, 2004, and 60/618,746, filed on Oct. 14, 2004. U.S. Provisional Application Nos. 60/535,360 and 60/618,746 are incorporated by reference herein in their entireties.

### II. BACKGROUND

Thrombin is a serine proteinase whose diverse substrates are situated at key points in a large number of physiologically critical pathways. Both the activation of thrombin and its enzymatic activity are highly regulated at multiple levels. The generation of active thrombin from its inactive zymogen prothrombin is mediated by the extrinsic and intrinsic coagulation pathways, which converge on factor Xa, and are under the control of the protein C, TFPI, and antithrombin III systems. Among thrombin's myriad functions are the activation of platelets, the polymerization of fibrin, activation of PAR (protease activated receptor) signaling, and the stimulation of cell proliferation. Significantly, each of these activities contributes to the pathogenesis of thrombotic, occlusive, and restenotic disorders in the native vasculature and on medical implants, which are used with increasing frequency. Thus, thrombin and its common pathway activator, factor Xa (fXa), are important therapeutic targets for efforts to reduce cardiovascular and device thrombosis and their serious impact on morbidity, mortality, and health care costs.

Activation of thrombin and its subsequent catalysis of downstream platelet activation, coagulation, signaling, and proliferative reactions occurs mainly on vascular, cell, and microparticle surfaces and in a narrow diffusion layer extending from them. Low molecular weight direct thrombin inhibitors can access this interfacial compartment, but have limited utility as antithrombotics because antithrombotic benefits require high systemic doses, which can also induce bleeding. Two endogenous plasma proteins, antithrombin III (ATIII) and heparin cofactor II, inhibit thrombin (and fXa in the case of ATIII), and have glycosaminoglyacan binding sites that allow them to bind to and accumulate on vascular surfaces and in underlying tissue. Furthermore, rates of ATIII inhibition of activated coagulation factors are accelerated by binding to heparin and/or heparan sulfate proteoglycans (HSPGs).

Disclosed herein are variants of ATIII that preferentially bind heparin and HSPG under static, low, and high wall shear rate conditions, such as when present in vasculature and mechanical vascular pieces, such as grafts, stents, ventricular assist devices, catheters or tubing. Also disclosed are methods of using these variants in situations where no-flow, low and high wall shear rates are present, as well as methods for identifying improved variants for heparin binding under no-flow, low and high wall shear rate conditions.

### III. SUMMARY

In accordance with the purposes of the disclosed materials, compounds, compositions, articles, and methods, as embodied and broadly described herein, the disclosed subject matter, in one aspect, relates to compounds and compositions and methods for preparing and using such compounds and compositions. In another aspect, the disclosed subject matter relates to methods and compositions related to using variants of ATIII that have high affinity for heparin and heparan sulfate proteoglycans under no-flow, low and high wall shear rate conditions.

The advantages described below will be realized and attained by means of the elements and combinations particularly pointed out in the appended claims. It is to be understood that both the foregoing general description and the following detailed description are exemplary and explanatory only and are not restrictive.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a diagram of an in vitro system for mimicking the conditions of a subject's circulatory system.

FIG. 2 shows the loading of endogenous ATIII isoforms onto heparin-coated surfaces under low and high wall shear rate conditions. Panel A is an SDS gel showing alpha- and beta-ATIII remaining in the fluid phase and bound to the surface of various segments of the in vitro system of FIG. 1 after 0, 3, and 120 minutes of recirculation. Panel B shows quantitation of surface-bound ATIII (ng/segment, avg±SD) for low (light gray) and high (dark gray) wall shear rate sections of the FIG. 1 circuit following 0, 3, and 120 minutes of recirculation with a sample containing 1 μM each alpha and beta ATIII.

FIG. 3 shows flow effects on the loading of recombinant DES.N135A.ATIII onto heparin-coated surfaces. Panels A and B show surface-bound ATIII (ng/segment, avg±SD) for heparin-coated tubing exposed to plasma±DES.N135A ATIII under low (light gray) and high (dark gray) wall shear rate conditions. Panel C shows an SDS-PAGE of ATIIIs loaded onto the lumenal surface of heparin-coated tubing during 3 m recirculation. The “pre” lane contains the pre-circulation sample of diluted plasma (50%) plus 1 μM DES.N135A. Only the most abundant protein components of the sample (*, albumin; #, antitrypsin and Ig light chains; =, haptoglobin β chain) show up on the gel. Low and high wall shear rate segments are marked as in FIG. 1.

FIG. 4 shows a diagram of an in vitro system for mimicking the conditions in a subject's circulatory system.

FIG. 5 shows the relative amounts of ATIII isoforms eluted from various sections of the tubing in the system shown in FIG. 4 after four hours exposure to a sample containing equal parts of the alpha- and beta-isoforms under the indicated flow conditions. Isoform composition of the fluid phase prior to (lane a) and post circulation (lane b) is also shown.

FIG. 6 shows the relative amounts of ATIII alpha and beta isoforms loaded onto tubing surfaces of the FIG. 4 circuit following exposure under different flow conditions and for various times to a sample containing ATIII isoforms in the plasma ratio of >90:<10.

FIG. 7 shows the relative amounts of alpha- and beta-ATIII loaded onto tubing surfaces of the FIG. 4 circuit following 15 minutes exposure at the indicated wall shear rates to a sample containing the ATIII alpha and beta isoforms and DES.N135A (a recombinant ATIII with enhanced binding affinity for heparin) in an about 40:40:20 ratio. Also shown are the ATIII compositions of the fluid phase prior to (lane a) and after (lanes b and c) recirculation.

FIG. 8 shows a CIRCUIT constructed from 1.6 mm ID uncoated and 3.0 mm ID CBAS™ (Carmeda Bioactive Surface) heparin-coated PVC (polyvinylchloride) tubing. The FLOW RATE: was Q=7 mL/min, and the WALL SHEAR RATES were: section A, 44 sec⁻¹ to (venous); section B, 2,000 sec⁻¹ (arterial); section C, 15,000 sec⁻¹ (pathological). This circuit was used for the experiments shown in FIGS. 9 and 10, in which ATIII containing samples flowed through the loop for various lengths of time. After recirculation, the fluid phase containing unbound ATIII was collected and the circuit washed with buffered saline. The CBAS™ tubing sections A, B, and C were then each cut into three 2-cm segments. Surface-bound ATIII was eluted from the tubing pieces, and the isoform content determined by 10% SDS-PAGE.

FIG. 9 shows the results of an experiment in which a sample containing 1 μM each human plasma-derived alpha and beta ATIII isoforms was circulated through the FIG. 8 circuit. The Panel A SDS-PAGE gels shows wall shear rate effects on ATIII isoform binding to heparin-coated surfaces after 3 or 120 minutes of recirculation. The “Fluid phase-pre-circ” lanes show the initial 1:1 alpha:beta ratio of injected sample. The “Surface-bound ATIII lanes” show more rapid binding of beta-ATIII, especially at the higher WSRs encountered during arterial and pathological flow. Progressive depletion of beta-ATIII relative to alpha-ATIII is observed in 3 min and 120 min “fluid phase-post-circ” samples. Panel B shows quantitative analysis of information from the panel A gels. For WSRs of 44 to 15,000 sec⁻¹, beta-ATIII bound to the heparin-coated surface more rapidly than alpha-ATIII. At the arterial and pathological WSRs, initial (3 min) rates of beta isoform loading were twice that at the venous WSR. In contrast, rates of alpha isoform loading were WSR-independent. The panel C plot shows total (alpha plus beta) surface-bound ATIII as function of wall shear rate and exposure time. Initial rates of ATIII binding to the heparin-coated biomaterial surface were faster in higher WSR sections of the circuit. At “equilibrium” (120 min), the amounts of surface-bound ATIII were similar for all wall shear rates.

FIG. 10 shows the results of an experiment in a 50% solution of human plasma containing approximately 1 μM ATIII (about 90% alpha and about 10% beta) supplemented with 1 μM DES.N135A ATIII and recirculated through the FIG. 8 circuit. DES.N135A is a recombinant ATIII that binds heparin with 50 times higher affinity than alpha-ATIII (the major isoform in plasma), and with 10 times higher affinity than beta ATIII (the minor isoform in plasma). The panel A SDS-PAGE gels show that the rate of DES.N135A surface binding exceeded the rate plasma ATIII binding, and that this effect was strongest at the higher wall shear rates. At “equilibrium” (120 min), most of the surface-bound ATIII was DES.N135A ATIII, rather than endogenous plasma-derived ATIII. Panel B is quantitive analysis of data from the panel A gels, and shows the rate of plasma ATIII (mostly alpha ATIII) loading onto the heparin coated biomaterial surface was largely independent of WSR. Recombinant DES.N135A loaded onto the surface 2×, 5×, and 7× faster than plasma ATIII at WSRs of 44, 200, and 15,000 sec⁻¹, respectively. Under venous, arterial, and pathological flow conditions, supplementing plasma with 1 μM recombinant DES.N135A ATIII lead to >10-fold increases in the amount of surface bound ATIII.

FIG. 11 shows an in vitro flow model experiment demonstrating functional inhibition of flowing thrombin by surface-targeted ATIIIs. The experimental protocol involved the injection of 50% human plasma supplemented with saline (control) or ATIII into the circuit shown in panel A followed by recirculation for 15 min at flow rates producing wall shear rates (WSRs) of 150 or 2,000 sec⁻¹. Then the circuit washed with normal saline, and 10 nM human thrombin was injected and recirculated for 15 min. Finally, the fluid phase was recovered and residual thrombin enzymatic activity was measured by chromogenic assay. Panel B shows the results of this experiment including “No addition” controls in which the circuit was exposed to unsupplemented human plasma at WSRs of 150 or 2,000 sec⁻¹ producing, respectively, 40% and 55% thrombin inhibition. Exposure of the circuit to human plasma supplemented with up to 3 μM purified plasma-derived ATIII (triangles) (which is mostly alpha isoform (see gel lane 2)) produced further small increases in the levels of thrombin inhibition. Supplementation with lesser concentrations of beta-ATIII-enriched (squares) (see gel lane 3) or recombinant DES.N135A (round light grey dots) (see gel lane 4) produced more thrombin inhibition than supplementation with higher concentrations plasma-derived ATIII. Supplementation with 1 μM beta-enriched or DES.N135A ATIIIs produced 65% thrombin inhibition at the venous WSR of 150 sec⁻¹. Supplementation with 1 μM beta-enriched or DES.N135A ATIIIs produced >80% thrombin inhibition at arterial and pathological WSRs of 2,000 and 15,000 sec⁻¹.

FIG. 12 shows the structures of native ATIII and an ATIII-pentasaccharide bound activated complex in the region between the heparin binding site and the reactive center loop. Panel A shows native ATIII, AT^{N}, drawn from 1E05i.pdb. The reactive center loop (RCL with P14 serine 380 side chain shown) is inserted between strands 2A/3A and 5A/6A of central beta sheet A. Helix D and the polypeptide amino terminal to it contain heparin binding site residues arg-129, lys-125, phe-122, and lys-114 (shown). A tight cluster composed of residues from helix D and strand 2A is organized around the ring of tyrosine-131, which originates from the polypeptide C-terminal to helix D. Panel B shows pentasaccharide bound and activated ATIII, AT*H, drawn from 1E03i.pdb. Pentasaccharide occupancy of the heparin binding site leads to P helix formation in the polypeptide that is N-terminal to helix D, and the C-terminal extension of helix D by one turn. The associated rotation of L130 and Y131 breaks up the cluster centered around the Y131 ring in native ATIII. The Y131-L140 CA helix D to s2A distance increases from 5.8 to 7.7 Å, with a reciprocal reduction of 7.6 to 4.8 Å in the CA spacing between Y220 and F372, which bridge the s2A/s3A from s5A/s6A gap. The reactive center loop is expelled from the A sheet. Panel C shows a table that documents pentasaccharide mediated disruption of the native Y131 cluster as increases in distances between Y131 distal ring carbons and helix D and strand 2A residues of the ATN and AT*H conformations.

FIG. 13 shows Laemmli gels of surface-bound and post-binding fluid phase samples 3, 30, or 180 minutes after plasma derived t.ATIII or recombinant DES.N135A ATIII loading.

### V. DETAILED DESCRIPTION

The materials, compounds, compositions, articles, and methods described herein may be understood more readily by reference to the following detailed description of specific aspects of the disclosed subject matter and the Examples included therein and to the Figures.

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

A. Definitions

In this specification and in the claims which follow, reference will be made to a number of terms which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15.

References in the specification and concluding claims to parts by weight of a particular element or component in a composition or article denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

“Optional” or “optionally” means that the subsequently described event or circumstance may or may not occur, and that the description includes instances where said event or circumstance occurs and instances where it does not.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

As used herein, by a “subject” or “patient” is meant an individual. Thus, the “subject” or “patient” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” or “patient” can also include a mammal, such as a primate or a human.

Reference will now be made in detail to specific aspects of the disclosed materials, compounds, compositions, articles, and methods, examples of which are illustrated in the accompanying Examples and Figures.

B. Compositions and Methods

Antithrombin III (ATIII) is a 60,000 kDa endogenous serine proteinase inhibitor (serpin) that prevents excessive clotting in blood and on lumenal surfaces of the circulatory system. The anticoagulant activity of pharmaceutical heparin derives from its ability to serve as a cofactor for antithrombin III by greatly accelerating ATIII-mediated inhibition of most coagulation factors, including thrombin and fXa, and factors IXa, XIa, XIIa (and its fragments) and plasma kallikrein-high molecular weight kininogen in the intrinsic pathway, and factor VIIa-TF in the extrinsic pathway. Thus, in addition to inhibiting thrombin as its name implies, ATIII also blocks most serine proteinases generated during activation of the clotting system, and is a powerful anticoaguant because it inhibits not only the enzymatic activity of thrombin, but also its generation. Moreover, in addition to activation of ATIII proteinase inhibitory functions, the binding interaction between heparin and ATIII also serves to target it to HSPG receptors on surfaces at the blood-vessel wall interface where thrombin is generated. As disclosed herein, this interaction plays a role in maintaining patency in high wall-shear-rate regions of the circulatory system.

The disclosed data indicate that ATIII-HSPG interactions are important under low and high shear rate conditions. High wall-shear-rate regions will become prone to thrombosis and inflammation when circulating beta-ATIII concentrations drop to levels that do not support sufficient loading of vascular wall HSPG receptors, and ATIII-dependent surface anticoagulant, anti-proliferation, NF-KB blocking, and prostacyclin releasing activities are reduced. The disclosed high affinity heparin binding ATIII and low-dose super beta-ATIIIs with enhanced-affinity for heparin/HSPG will be useful for maximizing surface-bound ATIII, especially in various high shear procedures such as angioplasty (with and without stent implantation), and during CPB and LVAD support. As used herein, “super beta” or any form of this, refers to ATIII molecules that have an affinity for heparin that is greater than the affinity for heparin of the human plasma-derived beta ATIII isoform, and a beta ATIII is an ATIII that is not gycosylated at N135 or its positional equivalent amino acid in an ATIII. A positional equivalent amino acid is an amino acid that is performing the same function as a specific amino acid in a peptide, but which may have a different position in the primary sequence of the peptide due to deletions or additions that may have occurred N-terminal to the specific amino acid. For example, naturally occurring ATIII has a position, N135, which can be glycosylated. This asparagine typically resides at position 135 of an ATIII, but, if for example, the first N-terminal amino acid was removed, this specific amino acid would now be Asparagine 134. Asparagine 134 in this example would be a positional equivalent amino acid.

Thrombotic and inflammatory reactions are significant clinical problems following balloon angioplasty with and without stent placement. Even using CBAS™ heparin-coated stents about 12% of patients in the Benestent II, Pani/Stent and Tosca trials still failed to achieve sustained patency (Kocsis, et al., (2000) J of Long-Term Effects of Medical Implants 10, 19-45). Outcomes for these patients can be improved by preloading the stents with super beta-ATIII and/or a period of adjunctive treatment with super beta-ATIII.

Another category of patients that can benefit from super beta-ATIII treatment are those who have been implanted with non-pulsatile continuous flow centrifugal or impeller driven left ventricular assist devices that have heparin-coated interior surfaces. Approximately one third (8/22) of patients with Micromed DeBakey LVAD implants developed low blood flow rates and increased power consumption indicative of intrapump thrombosis, and required emergency thrombolytic treatment to restore flow (Rothenburger, et al., (2002) Circulation 106 [suppl I], I-189-92). Adjunctive treatment with low dose, enhanced-heparin-affinity super beta-ATIIIs, especially during the period immediately following implantation, can be useful for boosting antithrombotic and anti-inflammatory activities on the patients own vascular surfaces and on surfaces of the device.

Heparin-coated circuits are widely employed in cardiovascular surgeries where on-pump CPB is utilized. In this context, adjunctive treatment with super beta-ATIII can reduce thrombosis and associated neurocognitive function and stroke problems, and can also decrease intraoperative heparin requirements and hemorrhagic risk. On-pump bypass patients frequently develop ATIII deficiencies in conjunction with systemic inflammation and elastase activity increases (Cohen, et al., (1992) J Invest Surg 5:45-9). Disclosed are recombinant antithrombins that are highly resistant to inactivation by neutrophil elastase and cathepsin G, and these variants can also be made on a super beta-ATIIIs backbone, which are being considered for use in settings that are inflammatory, as well as thrombotic.

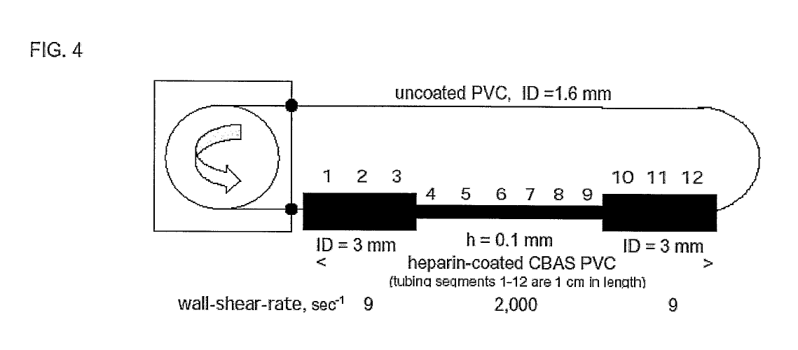

Adjunctive super beta-ATIII can be useful for improving the performance of CBAS™-coated ePTFE grafts in low, as well as high, wall-shear-rate contexts. Although CBAS™ coating of ePTFE vascular grafts improved their performance in a canine carotid artery model, there is still room for improvement above the 50% patency rate observed at 180 days post implantation (Begovac, et al., (2003) Eur J Vasc and Endovasc Surg 25:432-7). The experiment shown in FIG. 4 illustrates dramatic targeting of DES.N135A, a prototypically super beta-ATIII, to the high wall-shear-rate section of a CBAS™-coated circuit. However, this enhanced-affinity ATIII also preferentially bound low wall shear-rate segments as well. This observation demonstrates additional potential benefits of super beta-ATIII in applications targeting low wall-shear-rate targets.

As disclosed herein, antithrombin III isoforms flowing through CBAS™ tubing indicate that evolutionary conservation of the production of two glycoforms facilitates partitioning of ATIII antithrombotic and anti-inflammatory activities between the circulating blood and vascular wall surfaces. The higher heparin affinity of the beta-ATIII isoform allows it to effectively bind to and protect low and high shear rate sections of the circulatory system, despite its relatively low concentration in blood. These findings provide paradigms for investigating and understanding ATIII-vascular surface interactions, as wells as strategies for the development of low dose super beta-ATIII to increase the antithrombotic and anti-inflammatory properties of vascular surfaces and heparin-coated medical devices.

The production of antithrombin III isoforms with different heparin/HSPG affinities is evolutionarily conserved. The beta-ATIII isoform preferentially associates with HSPG receptors on vascular surfaces and may primarily mediate surface anticoagulant, antithrombotic and antiinflammatory reactions, whereas the principal function of the alpha-ATIII isoform may be to prevent stasis-associated (venous) thrombosis in the blood. These considerations suggest that beta-ATIII interactions with vascular surfaces warrant further investigation, and in particular, that studies under physiologically realistic flowing conditions should be performed. Greater understanding of beta-ATIII-vascular surface interactions may lead to the development of beta-ATIII and beta-ATIII derivative-based strategies to more effectively block pathologic thrombotic and inflammatory reactions on the vessel wall and in heparin-coated medical devices.

It is also understood that the data disclosed herein indicates that variant ATIIIs as disclosed herein are useful to be administered to subjects who have high wall shear stress rates in one or more vessels, such as rates greater than about 2000 sec⁻¹, for example.
1. ATIII Binds Vasculature Surfaces

The inhibitory activity of ATIII towards its target enzymes is enhanced by heparin (Rosenberg and Damus, (1973) J Biol Chem 248:6490-6505) and vascular surface heparan sulfate proteoglycans (HSPGs) (Marcum, et al., (1983) Am J Physiol 245:H725-733). The heparin binding property of antithrombin directs ATIII to sites where its target enzymes are generated, and potentiates its activity on these surfaces. Thus heparin upregulates the inhibitory activity of ATIII, and also spatially regulates it so that highest rates of thrombin and factor Xa inhibition are achieved on heparan sulfate proteoglycan (HSPG)-containing vascular surfaces.

It is generally believed that activated ATIII molecules bound on vascular surface HSPG receptors contribute substantively to the anticoagulant and antithrombotic properties of the endothelium (deAgostini, et al., (1990) J Cell Biol 111:1293-1304). This view is supported by a recent report of lethal thrombosis in mice homozygous for an ATIII mutation that blocks binding to heparin/HSPG (Dewerchin, et al., (2003) Circ Res 93). Similarly, the high-affinity fraction of heparin that binds ATIII is required to prevent thrombosis in stents implanted in baboon extracorporeal arterio-venous shunts (Kocsis, et al., (2000) J of Long-Term Effects of Medical Implants 10:19-45). Therefore, antithrombin-vascular surface interactions play a critical role in maintaining circulatory system patency, and a previous contrary report based on the non-thrombotic phenotype of mice deficient for 3-OST-1 (HajMohammadi, et al., (2003) J Clin Invest 111:989-99) may reflect redundancy of enzymes that mediate 3-0 sulfation of the pentasaccharide sequence of HSPG (Weitz, (2003) J Clin Invest 111:952-4).

Although ATIII binding to vascular surfaces has been primarily investigated in the context of coagulation inhibition and thrombosis, recent studies indicate that this interaction also modulates anti-inflammatory properties of the endothelium. ATIII binding to HSPGs on endothelial cells or neutrophils promotes release of anti-inflammatory prostacyclin and blocks activation of proinflammatory NF-KB, which in turn leads to decreased platelet and neutrophil activation, chemotaxis, and interaction with the endothelium. These effects disappeared when the experiments were conducted with antithrombin that had been blocked in the heparin binding domain (Dunzendorfer, et al., (2001) Blood 97:1079-85; Hoffmann, et al., (2002) Crit Care Med 30:218-25; Oelschläger, et al., (2002) Blood 99:4015-20).
2. Glycosylation Isoforms of ATIII

Human plasma contains two ATIII isoforms due to partial glycosylation of asparagine-135 (Peterson and Blackburn, (1985) J Biol Chem 260:610-5; Picard, et al., (1995) Biochemistry 34:8433-40). The minor beta ATIII isoform constitutes approximately 10% of the ATIII in blood and binds heparin with 5-fold greater affinity than the major alpha isoform (Turk, et al., (1997) Biochemistry 36:6682-91). Several kinds of evidence suggest that the beta isoform is primarily 50 responsible for thrombin and activated clotting factor inhibition in the critical blood-vessel wall interfacial region where occlusive restonotic clotting and intimal hyperplasia reactions originate (Carlson, et al., (1985) Biochem J 225:557-64; Witmer and Hatton, (1991) Arteriosclerosis and Thrombosis 11:530-9; Frebelius, et al., (1996) Thromb Vasc Biol 16:1292-7). As disclosed herein, binding of ATIII isoforms to a heparin-coated surface under flowing conditions also imply that the beta isoform plays a critical role in thrombin and fXa inhibition on vascular surfaces.

Under no-flow conditions, differential glycosylation of the isoforms is responsible for a 6-fold difference in their affinity for the ATIII cofactor/receptor, heparin/HSPG (Turk, et al., (1997) Biochemistry 36:6682-91).

The origin of glycosylation differences between the alpha and beta ATIII isoforms is synthetic rather than degradative. Partial modification of asparagine 135 occurs due to the presence of a serine, rather than a threonine, in the third position of its N-glycosylation consensus signal, leading to 50:50 production of alpha and beta molecules (Picard, et al., (1995) Biochemistry 34:8433-40). Two isoforms of ATIII are produced because ATIII has a serine, instead of a threonine, in the third position (S137) of its N135 tripeptide consensus sequence for N-glycosylation. This causes partial glycosylation at asparagine 135, in which N135 is glycosylated for some molecules and not glycosylated for others. Beta-ATIII molecules result from failure to glycosylate on N135. Alpha ATIII molecules are those which have been glycosylated on N135. The N135 glycosylation or the failure to modify N135 occurs on a background of essentially full modification at three other ATIII N-glycosylation sites (N96, N155 and N192). Alpha-ATIII typically has four N-linked oligosaccharides, and beta-ATIII has only three. The body typically does not synthesize any totally “unglycosylated” ATIII. The N-X-S tripeptide consensus sequence encoding the production of alpha and beta glycoforms is conserved evolutionarily in vertebrates having 3 and 4 chambered hearts (mammals, birds, reptiles and amphibians) (Backovic and Gettins (2002) J Proteome Res 1:367-73). This evolutionary conservation suggests that it is advantageous to carry two different antithrombin isoforms, and that each one has a distinct and critical function. In contrast, fish make only the beta-ATIII isoform, which may be related to fundamental differences in fish circulatory systems, which have 2-chambered hearts.

Although alpha- and beta-antithrombin are synthesized in a 50:50 ratio (Picard, et al., (1995) Biochemistry 34:8433-40; Bayston, et al., (1999) Blood 93:4242-7), they circulate in mammalian blood in a ratio of approximately 90alpha:10beta. The beta isoform clears more rapidly from the blood (Carlson, et al., (1985) Biochem J 225:557-64) and occurs at higher §0 concentrations relative to alpha-ATIII in antithrombin eluted from rabbit aorta intima/media (Witmer and Hatton (1991) Arteriosclerosis and Thrombosis 11:530-9). Moreover, beta-ATIII, but not alpha-ATIII, reduced surface thrombin activity following balloon injury of rabbit aorta (Frebelius, et al., (1996) Thromb Vasc Biol 16:1292-7).

Disclosed herein, experiments proving that the beta-isoform preferentially associates with HSPG receptors of the vascular endothelium under high wall shear rates, and beta ATIIIs and other molecules disclosed herein provide the desired activity of binding the heparin and HSPG attached to walls. This data indicate that high affinity heparin binding, such as the beta-isoform possesses, play an important role in anticoagulant, antithrombotic and anti-inflammatory reactions on these surfaces.
3. ATIII is Negatively Regulated by Proteases and Elastases

ATIII is negatively regulated in part by elastases and proteases that cleave ATIII, preventing ATIII from inhibiting thrombin and factor Xa. Human neutrophil elastase cleaves and inactivates ATIII (Jochum, et al., (1981) Hoppe-Seyler's Z Physiol Chem 362:103-12). The reported neutrophil elastase cleavage sites were after the P5-Val and P4-IIe in the reactive loop of ATIII (Carrell and Owen, (1985) Nature 317:730-2). Furthermore, Jordan and colleagues showed that elastase inactivation of ATIII was heparin dependent (Jordan, et al., (1987) Science 237:777-9). It has been hypothesized that elevated elastase (Nuijens, et al., (1992) J Lab Clin Med 119:159-68) is responsible for the inactivation of ATIII in sepsis (Seitz, et al., (1987) Eur J Haematol 38:231-40) and reduced antithrombin levels in septic disseminated intravascular coagulation (DIC) (Bick, et al., (1980) Am J Clin Path 73:577-83; Buller and ten Cate, (1989) Am J Med 87:44 S-48S; Damus and Wallace, (1989) Thromb Res 6:27; Hellgren, et al., (1984) Intensive Care Med 10:23-8; Lammle, et al., (1984) Am J Clin Pathol 82:396-404; Mammen, et al., (1985) Semin Thromb Hemost 11:373-83). Also included is the condition where cardiopulmonary bypass has increased elastase levels. (Cohen, et al., (1992) J Invest Surg 5:45-9, which is herein incorporated by reference at least for material related to heparin and bypass surgery.)

Originally, the term protease referred to enzymes that cleaved the peptide bonds of low molecular weight polypeptides, and the term proteinase referred to enzymes that cleaved the peptide bonds of higher molecular weight proteins. More recently, the distinction between these two terms has become blurred in practical usage. In accordance with modern usage, this application also uses the term protease to refer to an enzyme that cleaves peptide bonds of proteins.

There are a variety of proteases that cleave the reactive loop of ATIII without the production of stable inhibitory complexes. These proteases can potentiate the expression of thrombin and fXa enxymatic activity by cleaving and inactivating the primary inhibitor of these coagulation factors, antithrombin III. Human neutrophil elastase (HNE) can cleave and inactivate ATIII. The primary cleavage sites for HNE are in the ATIII reactive loop, and their location can be described using the standard nomenclature of Schechter and Berger (Schechter and Berger, (1967) Biochem Biophys Res Commun 27:157-162, which is herein incorporated by reference at least for material related to ATIII cleavage and amino acid designations), wherein the amino acids of the reactive loop are referred to based on their location relative to the P1-P1′ peptide bond that is cleaved by the thrombin or factor Xa during inhibitory complex formation. Residues amino terminal to this bond are designated P2, P3, etc, and those on C terminal to it are designated P2′, P3′, etc. HNE inactivates ATIII by cleavage after its P5-Val and P4-IIe residues (Carrell and Owen, (1985) Nature 317:730-2, which is herein incorporated by reference at least for material related to ATIII cleavage and amino acid designations).

Those of skill in the art understand that different allelic variants of ATIII and different species variants of ATIII for example, have an analogous site, such as a positional equivalent amino acid, that is cleaved during inhibitory complex formation, and that this can readily be determined. Because the absolute position of this site in the numbered sequences of different ATIIIs can change, a standard nomencature is employed to designate the relationship of reactive loop amino acids to the point of cleavage during inhibitory complex formation. (Schechter and Berger, (1967) Biochem Biophys Res Commun 27:157-62). As discussed herein, antithrombin sequences disclosed herein can address this issue by referring to specific amino acids as positional equivalent amino acids as discussed herein.
4. Heparin/HSPG Activation of ATIIIs

In the absence of activating cofactors, ATIIIs are less efficient inhibitors of these target enzymes. The basal rate of inhibition in the absence of cofactors is referred to as “progressive” activity. Second order rate constants for progressive ATIII inhibition of thrombin and factor Xa are typically in the 10³ to 10⁴ M⁻¹sec⁻¹ range. These rates, however, typically are accelerated by a factor of tuting a thousand (i.e., into the 0.5×10⁶ to 10⁷ M⁻¹sec⁻¹ range) when certain kinds of sulfataed glycosaminoglycan cofactors (heparin or heparan sulfate proteoglycans (HSPG), low molecular weight heparins and synthetic heparins) bind to ATIII. Heparins are widely used pharmaceuticals that have been administered as anticoagulants since the 1940's, while heparan sulfate proteoglycans (HSPGs) serve as the physiological cofactor for ATIII. HSPGs on the vascular endothelim and in the underlying matrix present heparin-like molecules to circulating blood and serve to localize and activate ATIII on surfaces where coagulation enzymes are generated.

As noted, ATIII is an endogenous anticoagulant serpin that inhibits activated blood coagulation enzymes using a suicide substrate mechanism. The process of proteinase inhibition by serpins is initiated when a target enzyme cleaves the exposed reactive center loop (RCL) of the serpin to generate a covalent acyl-enzyme complex in which the cleaved RCL polypeptide becomes incorporated into the central A beta sheet of the serpin (Lawrence, et al., (1995) J Biol Chem 270:25309-12; Wilczynska, et al., (1995) J Biol Chem 270:29652-5). In the inhibitory complex, the target proteinase has been translocated some 70 Å from its original docking site on the serpin, and is inactivated by catalytic triad and general structural distortion, which prevent deacylation (Huntington, et al., (2000) Nature 407:923-6). Most serpins inhibit their target enzymes at essentially diffusion-limited rates (about 10⁷ M⁻¹sec⁻¹), however, antithrombin III is an exception. In the absence of cofactors ATIII inhibits its target enzymes at rates that are 3-4 orders of magnitude slower than the rapid inhibition rates achieved by most other serpins Olson, et al., (2004) Thrombosis and Haemostasis 92(5):929-39. The poor inhibitory function of ATIII is due to a structural idiosyncrasy. In contrast to other serpins that have fully exposed reactive loops, native ATIII is a self-constrained molecule (Schreuder, et al., (1994) Struct Biol 1:48-54; Jin, et al., (1997) Proc Natl Acad Sci USA 94:14683-8; Skinner, et al., (1997) J Mol Biol 266:601-9). Its reactive loop is partially inserted into its central A beta sheet, preventing target enzyme access to the scissile bond that is cleaved during suicide inhibition. This constraint is relieved by an uncatalyzed equilibrium between the native molecule and a cofactor-independent activated ATIII conformation that results in thrombin and fXa inhibition rates in the 10³ to 10⁴ M⁻¹sec⁻¹ range. The reactive loop constraint may also be released by native ATIII binding to a specific pentasaccharide component of pharmaceutical heparin and vascular wall heparan sulfate proteoglycans. Cofactor binding generates an activated, RCL-expelled, binary antithrombin-heparin complex (AT*H) that inhibits ATIII target enzymes at about 10⁷ M⁻¹sec⁻¹.

The crystal structures for native ATIII and AT*H conformations show that pentasaccharide binding is associated with elongation of alpha helix D, which contains several heparin binding residues (Desai, et al., (2000) J Biol Chem 275:18967-84; Schedin-Weiss, et al., (2002) Biochemistry 41:4779-88; Jairajpuri, et al., (2003) J Biol Chem 278:15941-50; Arocas, et al., (2000) Biochemistry 39:85 12-8; Meagher, et al., (1996) J Biol Chem 271:29353-8). Functional studies of helix D-s2A linker polypeptide deletion mutants (Meagher, et al., (2000) J Biol Chem 275:2698-704) and a K133P mutant (Belzar, et al., (2002) J Biol Chem 277:8551-8) also support a role for helix D (hD) elongation in heparin activation of ATIII anticoagulant activity. Factor Xa inhibition rates of the hD-s2A deletion mutants and K133P were similar to the parental control in the absence of cofactor, but about 10-fold lower than control in the presence of pentasaccharide or heparin.
5. Exemplary Types of ATIII
   a) ATIIIs with high affinity for bound heparin or HSPGs

According to the composition and methods disclosed herein, it can, in one aspect, be desirable to have ATIIIs which bind heparin or HSPGs with high affinity when the HSPG or heparin are bound to a surface, such as a vasculature surface or a mechanical surface, such as a stent or tubing. For example, the measurements of alpha, beta, and recombinant antithrombin loading onto surface bound heparin at different shear rates, disclosed herein, are useful for developing an improved and more physiologically accurate understanding of ATIII-HSPG regulatory interactions. In addition, knowledge of how ATIII binding affinities vary over the range of physiologically and pathologically relevant flow conditions can expedite the design and development of strategies utilizing low-dose, high-affinity recombinant antithrombins to efficiently block thrombin generation and inflammatory reactions on vascular and biomedical device surfaces. Physiological wall shear rates in the human circulatory system range from near to <50 sec⁻¹ in sinuses and some veins, to 500 to 5000 sec⁻¹ in arterioles of the normal circulation. Pathological wall shear rates (e.g., at top of plaques in 50% occluded arteries) are in the 3000 to 10,000 sec⁻¹ range. The calculations in Table 1 show the range of wall shear rates that can be achieved in in vitro flow model experiments utilizing reasonable amounts of antithrombins using 0.8 mm ID CBAS™ heparin-coated PVC tubing and experimentally accessible flow rates. It is of basic and therapeutic relevance to understand the kinetics of ATIII isoform and recombinant ATIII variant loading onto heparin/HSPG coated surfaces under flow conditions producing wall-shear-rates in the 50 to 3500 sec⁻¹ range.

TABLE 1
Calculation of wall shear rates as a function of of tubing
internal diameter and volumetric flow rate

| pump tubing ID | 1.6 mm | 3.2 mm | | |
|---|---|---|---|---|
| Q range, ml/min | 05-2 | 0.5-15 | | |
| CBAS ID | radius | Q | Q | γw |
| mm | mm | ml/min | mm\hat{ }3/sec | sec\hat{ }−1 |
| 0.8 | 0.4 | 0.2 | 3 | 66 |
| 0.8 | 0.4 | 2 | 33 | 663 |
| 0.8 | 0.4 | 5 | 83 | 1659 |
| 0.8 | 0.4 | 12 | 200 | 3981 |

shear rates in Ruggieri platelet studies: 50, 630, 1500 sec⁻¹
highest shear rates in normal ciculation = arterioles: 500-5000 sec⁻¹
shear rates at top of plaques in 50% occluded artery: 3000-10,000 sec⁻¹
wall-shear-rate = γw = 4Q/π R\hat{ }3
Q = volumetric flow rate,
R = radius at wall of tube

The K_{d}s for any ATIII molecule and the surface bound heparin or HSPG can be achieved by complementary equilibrium binding and association/dissociation rate strategies. The K_{d}s can be measured at wall-shear-rates of, for example, 50, 630 and 1500 sec⁻¹, which are the standard values used by Ruggieri's group for investigations of platelet adhesion under flow. Measurements can also be conducted at, for example, 3500 sec⁻¹ since wall-shear-rates of this magnitude have been measured in 50% stenosed arteries, which are also of interest as potential super beta-ATIII targets. The disclosed calculations indicate that rheologically relevant studies can be conducted using 0.8 mm ID CBAS™ tubing and quantities of plasma-derived ATIII isoforms and recombinant ATIIIs that can be realistically produced (see Table 1).
(1) Super Beta ATIIIs

Certain variant ATIIIs with increased affinity for heparin are disclosed in U.S. Pat. No. 5,700,663, which is herein incorporated by reference at least for ATIII variants. Variants disclosed are those that contain amino acid substitutions at position 49, 96, 135, 155, 192, 393, or 394 of SEQ ID NO:1.

Certain other variant ATIIIs with increased affinity for heparin are disclosed in U.S. Pat. No. 5,420,252 which is herein incorporated by reference at least for ATIII variants. Variants disclosed are those that contain amino acid substitutions in ATIII at positions 11 to 14, 41 to 47, 125 to 133, and 384 to 398 are substituted by another amino acid(s) such as Ala, Gly, Trp, Pro, Leu, Val, Phe, Tyr, IIe, Glu, Ser, Gln, Asn, and Arg.

Also disclosed are variants of ATIII and the use of these variants where the K_{d} for heparin is less than or equal to 0.01 nM, 0.05 nM, 0.1 nM, 0.5 nM, 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 51 nM, 52 nM, 53 nM, 54 nM, 55 nM, 56 nM, 57 nM, 58 nM, 59 nM, 60 nM, 61 nM, 62 nM, 63 nM, 64 nM, 65 nM, 66 nM, 67 nM, 68 nM, 69 nM, 70 nM, 71 nM, 72 nM, 73 nM, 74 nM, 75 nM, 76 nM, 77 nM, 78 nM, 79 nM, 80 nM, 81 nM, 82 nM, 83 nM, 84 nM, 85 nM, 86 nM, 87 nM, 88 nM, 89 nM, 90 nM, 91 nM, 92 nM, 93 nM, 94 nM, 95 nM, 96 nM, 97 nM, 98 nM, 99 nM, 100 nM, 101 nM, 102 nM, 103 nM, 104 nM 105 nM, 106 nM, 107 nM, 108 nM, 109 nM, 110 nM, 111 nM, 112 nM, 113 nM, 114 nM, 115 nM, 116 nM, 117 nM, 118 nM, 119 nM, 120 nM, 121 nM, 122 nM, 123 nM, 124 nM, 125 nM, 126 nM, 127 nM, 128 nM, 129 nM, 130 nM, 131 nM, 132 nM, 133 nM, 134 nM, 135 nM, 136 nM, 137 nM, 138 nM, 139 nM, 140 nM, 141 nM, 142 nM, 143 nM, 144 nM, 145 nM, 146 nM, 147 nM, 148 nM, 149 nM, 150 nM, 151 nM, 152 nM, 153 nM, 154 nM, 155 nM, 156 nM, 157 nM, 158 nM, 159 nM, 160 nM, 161 nM, 162 nM, 163 nM, 164 nM, 165 nM, 166 nM, 167 nM, 168 nM, 169 nM, 170 nM, 171 nM, 172 nM, 173 nM, 174 nM, 175 nM, 176 nM, 177 nM, 178 nM, 179 nM, 180 nM, 181 nM, 182 nM, 183 nM, 184 nM, 185 nM, 186 nM, 187 nM, 188 nM, 189 nM, 190 nM, 191 nM, 192 nM, 193 nM, 194 nM, 195 nM, 196 nM, 197 nM, 198 nM, 199 nM, 200 nM, 201 nM, 202 nM, 203 nM, 204 nM, 205 nM, 206 nM, 207 nM, 208 nM, 209 nM, 210 nM, 211 nM, 212 nM, 213 nM, 214 nM, 215 nM, 216 nM, 217 nM, 218 nM, 219 nM, 220 nM, 221 nM, 222 nM, 223 nM, 224 nM, 225 nM, 226 nM, 227 nM, 228 nM, 229 nM, 230 nM, 231 nM, 232 nM, 233 nM, 234 nM, 235 nM, 236 nM, 237 nM, 238 nM, 239 nM, 240 nM, 241 nM, 242 nM, 243 nM, 244 nM, 245 nM, 246 nM, 247 nM, 248 nM, 249 nM, 250 nM, 251 nM, 252 nM, 253 nM, 254 nM, 255 nM, 256 nM, 257 nM, 258 nM, 259 nM, 260 nM, 261 nM, 262 nM, 263 nM, 264 nM, 265 nM, 266 nM, 267 nM, 268 nM, 269 nM, 270 nM, 271 nM, 272 nM, 273 nM, 274 nM, 275 nM, 276 nM, 277 nM, 278 nM, 279 nM, 280 nM, 281 nM, 282 nM, 283 nM, 284 nM, 285 nM, 286 nM, 287 nM, 288 nM, 289 nM, 290 nM, 291 nM, 292 nM, 293 nM, 294 nM, 295 nM, 296 nM, 297 nM, 298 nM, 299 nM, 300 nM, 301 nM, 302 nM, 303 nM, 304 nM, 305 nM, 306 nM, 307 nM, 308 nM, 309 nM, 310 nM, 311 nM, 312 nM, 313 nM, 314 nM, 315 nM, 316 nM, 317 nM, 318 nM, 319 nM, 320 nM, 321 nM, 322 nM, 323 nM, 324 nM, 325 nM, 326 nM, 327 nM, 328 nM, 329 nM, 330 nM, 331 nM, 332 nM, 333 nM, 334 nM, 335 nM, 336 nM, 337 nM, 338 nM, 339 nM, 340 nM, 341 nM, 342 nM, 343 nM, 344 nM, 345 nM, 346 nM, 347 nM, 348 nM, 349 nM, 350 nM, 351 nM, 352 nM, 353 nM, 354 nM, 355 nM, 356 nM, 357 nM, 358 nM, 359 nM, 360 nM, 361 nM, 362 nM, 363 nM, 364 nM, 365 nM, 366 nM, 367 nM, 368 n™, 369 nM, 370 nM, 371 nM, 372 nM, 373 nM, 374 nM, 375 nM, 376 nM, 377 nM, 378 nM, 379 nM, 380 nM, 381 nM, 382 nM, 383 nM, 384 nM, 385 nM, 386 nM, 387 nM, 388 nM, 389 nM, 390 nM, 391 nM, 392 nM, 393 nM, 394 nM, 395 nM, 396 nM, 397 nM, 398 nM, 399 nM, 400 nM, 401 nM, 402 nM, 403 nM, 404 nM, 405 nM, 406 nM, 407 nM, 408 nM, 409 nM, 410 nM, 411 nM, 412 nM, 413 nM, 414 nM, 415 nM, 416 nM, 417 nM, 418 nM, 419 nM, 420 nM, 421 nM, 422 nM, 423 nM, 424 nM, 425 nM, 426 nM, 427 nM, 428 nM, 429 nM, 430 nM, 431 nM, 432 nM, 433 nM, 434 nM, 435 nM, 436 nM, 437 nM, 438 nM, 439 nM, 440 nM, 441 nM, 442 nM, 443 nM, 444 nM, 445 nM, 446 nM, 447 nM, 448 nM, 449 nM, 450 nM, 451 nM, 452 nM, 453 nM, 454 nM, 455 nM, 456 nM, 457 nM, 458 nM, 459 nM, 460 nM, 461 nM, 462 nM, 463 nM, 464 nM, 465 nM, 466 nM, 467 nM, 468 nM, 469 nM, 470 nM, 471 nM, 472 nM, 473 nM, 474 nM, 475 nM, 476 nM, 477 nM, 478 nM, 479 nM, 480 nM, 481 nM, 482 nM, 483 nM, 484 nM, 485 nM, 486 nM, 487 nM, 488 nM, 489 nM, 490 nM, 491 nM, 492 nM, 493 nM, 494 nM, 495 nM, 496 nM, 497 nM, 498 nM, 499 nM, or 500 nM.

The K_{d} values can be measured at physiological or non-physiological conditions. Traditionally, ATIIIs K_{d} values are reported a physiological ionic strength, pH, and temperature. In some instances, such as with high affinity ATIIIs, as disclosed herein, K_{d}s can be measured under non-physiological conditions. For example, due to the ionic component of the ATIII-heparin binding interaction, salt can be an important factor in the K_{d} measurement. Thus, an ionic strength of 0.3 can be used because it is a condition where comparison of alpha, beta, and super-beta measured K_{d}s, rather than extrapolated K_{d}s, are possible. In one aspect, the K_{d}s disclosed herein can be measured at pH 7.4, I=0.3, and 25° C.

In one aspect, the affinity of the ATIII disclosed herein can be at least 5×, at least 50×, at least 100×, or at least 250× that of the major plasma ATIII-alpha isoforms.

It is also understood that ATIIIs which are produced in insect expression systems produce ATIIIs having higher affinities for heparin than ATIIIs produced in other recombinant systems or from native plasma. For example, ATIII alpha from Hamster Cho expression system has a K_{d} of 63 nM and a K_{d} of beta of 18 nM. The K_{d}s of alpha and beta from an insect system are 8 and 1 nM respectively. This is because the insect expressed ATIIIs have smaller N-linked oligosaccharides than the other expression systems and other than plasma ATIII. The effect of these smaller chains is to increase binding affinity.

Also, as the shear rate increases the effective binding to wall bound heparin decreases more quickly for alpha-ATIII than for beta-ATIII due to mass transport and molecular binding affinity considerations

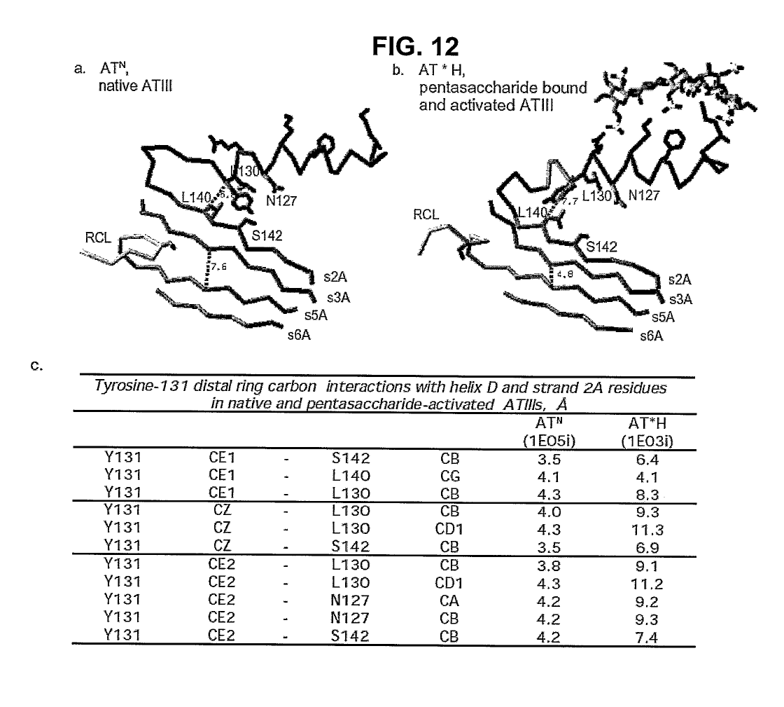

Also disclosed herein, the affinity of ATIII for heparin can be enhanced by disrupting structural interactions between helix D and strand 2A of its native conformation, which shifts the position of the equilibrium between the native reactive center loop-inserted conformation, ATN (FIG. 12a), and a cofactor-independent activated conformation. The equilibrium can be driven towards this activated conformation, which resembles AT*H, the heparin-bound and activated conformation of ATIII (FIG. 12b), by substituting non-phenylalanine amino acids for tyrosine-131 of human ATIII, or its positional equivalent in other antithrombins. As a result of the shifted conformational equilibrium, basal inhibition of fXa also increases. The increased heparin affinity of these variants can increase the efficiency of ATIII loading onto heparin- and HSPG-coated surfaces under static, and low and high wall shear rate flow conditions. The increased fXa inhibition activity of these variants can also provide improved regulation of systemic activated fXa.

“Super beta” ATIIIs are antithrombin molecules that bind heparin with greater affinity than the human plasma-derived beta ATIII isoform. The increased heparin affinities of recombinant super beta ATIIIs result from several kinds of modifications including, but not limited to: (1) disabling of N-glycosylation consensus sequences at N135, N96, N155, and/or N192, (2) synthesis by insect and yeast expression systems, which modify the protein with N-linked oligosaccharides that are smaller than those on human plasma-derived ATIII, and/or (3) modifications of ATIII RCL loop sequences (Jairajpuri, et al., (2002) J Biol Chem 277:24460; Rezaie, (2002) J Biol Chem 277:1235; Kato patent, P14, E380). The heparin affinities of such super beta ATIIIs can be even further enhanced by disrupting structural interactions between helix D and strand 2A of the native molecules to achieve additional improvements in ATIII surface targeting properties.
(2) Exemplary Super Beta ATIIIs

In some examples, disclosed herein are high affinity ATIIIs or super beta ATIIIs that have a substitution at tyrosine 131, or its positional equivalent in other antithrombins. Tyrosine 131 (Y131) is located in a polypeptide segment that is C-terminal to hD and that becomes alpha helical upon heparin binding. FIG. 12 shows that it undergoes a large rotation and dramatic shift in environment during the cofactor mediated activation process. Analysis of Y131 interactions in the crystal structures of native ATIII and the activated AT*H complex suggest two distinct roles for Y131 in the conformational regulation of ATIII by heparin. First, hydrophobic interactions of Y131 and surrounding hD and s2A residues (FIG. 12a) can contribute to stabilization of the native ATIII conformation. Secondly, heparin dependent rotation of the Y131 sidechain (FIG. 12b) can promote its use as a hD and s2A “spacer,” which in turn promotes closure of the s3A/s5A gap, and RCL expulsion.

Disclosed herein are ATIIIs variants with a substitution at Y131 or its positional equivalent amino acid where the substituted amino acid is capable of forming at least one contact (e.g., van der Waals or hydrophobic) with asn-127 and/or leucine-130, or their positional equivalent amino acid, in helix D. In another example, disclosed are ATIIIs with a substitution at Y131 or its positional equivalent amino acid where the substituted amino acid is capable of forming at least one contact with leucine-140 and/or serine-142, or their positional equivalents, in strand 2A. In still another example, disclosed are ATIIIs with a substitution at Y131 or its positional equivalent amino acid where the substituted amino acid is capable of forming a contact with asn-127, leucine-130, leucine-140, and serine-142, or their positional equivalents, including any combination thereof.

A contact as used herein means any position between two atoms, typically one atom of one amino acid to another atom of another amino acid in the disclosed compositions, such as between the Y131 and as-127 or its positional equivalent amino acid, that when positioned by an energy minimization program, for example, are less than 5 Å, 4 Å, 3 Å, 2 Å, or 1 Åapart. Thus, a contact can for example, correlate with, for example, non-covalent interactions, such as a hydrogen bonds, vander Waals interactions, hydrophobic interactions, and electrostatic interactions, between two atoms. Typically a contact will add to the binding energy between two atoms, but it can also be repulsive, typically more repulsive the closer the two atoms become. Although a contact is defined herein as being a relationship of two atoms, the molecules, amino acid residues, components, and compounds of which the atoms are a part can be referred to as having “contacts” with each other. Thus, for example, an amino acid having an atom that forms a contact with an atom in the strand 2A domain or helix D can be said to have a contact with the strand 2A domain or helix D. The contacts involved are the contacts between the atoms as described above.

A contact between atoms, molecules, components, or compounds is a form of interaction between the atom, molecules, components and compounds involved in the contact. Thus, an atom, molecule, component or compound can be said to “interact with” another atom, molecule, component, or compound. Such an interaction can be referred to at any level. Thus, for example, an interaction (or contact) between two atoms in two different molecules results in a relationship between the two molecules that can be referred to as an interaction between the two molecules containing the atoms. Similarly, an interaction between, for example, an inhibitor and an amino acid of a protein results in a relationship between the inhibitor and the protein that can be referred to as an interaction between the inhibitor and the protein. Unless the context clearly indicates otherwise, reference to an interaction between atoms, molecules, components or compounds is not intended to exclude the existence of other, unstated interactions between the atoms, molecules, components or compounds at issue or with other atoms, molecules, components or compounds. Thus, for example, reference to an interaction between one amino acid and another in ATIII does not indicate that there are not other interactions or contacts between the amino acids with other atoms, molecules, components, or compounds.

Unless the context clearly indicates otherwise, reference to the capability of atoms, molecules, components or compounds to interact with other atoms, molecules, components or compounds refers to the possibility of such an interaction should the atoms, molecules, components or compounds be brought into contact and not to any actual, presently existing interaction. Thus, for example, a statement that an inhibitor “can interact with” an amino acid of a protein refers to the fact that the inhibitor and amino acid would interact if brought into contact not that the inhibitor and amino acid are presently interacting.

In another aspect, the substituted amino acid at Y131, or its positional equivalent amino acid, can be oriented in the cleft between helix D and strand 2A in the native, cleaved, latent, latent-bound-to-pentasaccharide, and/or peptide complexed form, and flipped out of the pocket in the pentasaccharide-activated stated.

As described herein, the substitution at Y131, or its positional equivalent can be, any amino acid. In some examples, the substituted amino acid at position 131 (or its positional equivalent amino acid) can be alanine, arginine, asparagine, aspartic acid, cysteine, glutamic acid, glutamine, glycine, histidine, isoleucine, lysine, leucine, methionine, serine, threonine, tryptophan, or valine. It is also possible, in some aspects, to use phenylalanine and proline. In other examples, the substituted amino acid at position 131 (or its positional equivalent amino acid) can be a leucine, isoleucine, alanine, valine, or tryptophan.
6. Other Variants of ATIII

Other variants of ATIII disclosed herein are variants that have improved elastase and/or protease resistance while still retaining thrombin and/or fXa inhibition. These variants can also have improved heparin binding. These mutants can be found in U.S. Patent Application Nos. 60/085,197, 60/384,599, Ser. Nos. 09/305,588, 10/014,658, and PCT applications PCT/US99/10549 and PCT/US03/17506, which are herein incorporated by reference at least for material related to variants of ATIII. Disclosed are substitutions, wherein the substitution made at position P2, alone or collective with substitutions at either P3, P4, P5, P6, P7, and/or P8 or any other variants disclosed herein, is P.

Disclosed are substitutions, wherein the substitution made at position P3, alone or collective with substitutions at either P2, P4, P5, P6, P7, and/or P8 or any other variants disclosed herein, is D, E, G, H, I, K, L, N, P, Q, R, S, W, or Y.

Disclosed are substitutions, wherein the substitution made at position P3, alone or collective with substitutions at either P2, P4, P5, P6, P7, and/or P8 or any other variants disclosed herein, is D, E, H, K, L, P, Q, R, W, or Y.

Disclosed are substitutions, wherein the substitution made at position P4, alone or collective with substitutions at either P2, P3, P5, P6, P7, and/or P8 or any other variants disclosed herein, is A, F, G, L, N, P, Q, V, or W.

Disclosed are substitutions, wherein the substitution made at position P4, alone or collective with substitutions at either P2, P3, P5, P6, P7, and/or P8 or any other variants disclosed herein, is L, N, Q, V, or W.

Disclosed are substitutions, wherein the substitution made at position P5, alone or collective with substitutions at either P2, P3, P4, P6, P7, and/or P8 or any other variants disclosed herein, is E, F, G, P, D, S, T, N, Q, H, R, K, or V.

Disclosed are substitutions, wherein the substitution made at position P6, alone or collective with substitutions at either P2, P3, P4, P5, P7, and/or P8 or any other variants disclosed herein, is E, G, L, or T.

Disclosed are substitutions, wherein the substitution made at position P7, alone or collective with substitutions at either-P2, P3, P4, P5, P6, and/or P8 or any other variants disclosed herein, is E, N, Q, V, L, F, S, T, or H.

Disclosed are substitutions, wherein the substitution made at position P8, alone or collective with substitutions at either P2, P3, P4, P5, P6, P7, and/or P8, or any other variants disclosed herein, is E.

Disclosed are variants, having at least one substitution at position P2, P3, P4, P5, P6, or P7, wherein the substitution at P2 can be P, wherein the substitution at P3 can be D, E, G, H, I, K, L, N, P, Q, R, S, W, or Y, wherein the substitution at P4 can be A, F, G, L, N, P, Q, V, or W, wherein the substitution at P5 can be E, F, G, or P, wherein the substitution at P6 can be E, G, L, or T, wherein the substitution at P7 can be E or Q.

Disclosed are variants, having at least one substitution at position P2, P3, P4, P5, P6, or P7, wherein the substitution at P2 can be P, wherein the substitution at P3 can be D, E, H, K, L, P, Q, R, W, or Y, wherein the substitution at P4 can be L, N, Q, V, or W, wherein the substitution at P5 can be E or F wherein the substitution at P6 can be G or L, wherein the substitution at P7 can be E.

Disclosed are variants, having at least one substitution at position P2, P3, P4, P5, P6, or P7, wherein the substitution at P2 can be P, wherein the substitution at P3 can be D, E, G, H, I, K, L, N, P, Q, R, S, W, or Y, wherein the substitution at P4 can be A, F, G, L, N, P, Q, V, or W, wherein the substitution at P5 can be D, E, F, G, H, K, N, P, Q, R, S, T, or V wherein the substitution at P6 can be E, G, L, or T, wherein the substitution at P7 can be E, F H, I, L, N, Q, S, T, or V, or wherein the substitution at P8 can be E.

Disclosed are variants, having at least one substitution at position P2, P3, P4, P5, P6, or P7, wherein the substitution at P2 can be P, wherein the substitution at P3 can be D, E, H, K, L, P, Q, R, W, or Y, wherein the substitution at P4 can be L, N, Q, V, or W, wherein the substitution at P5 can be D, H, K, N, Q, R, S, T, or V wherein the substitution at P6 can be G or L, wherein the substitution at P7 can be F, H, L, S, T, V.

Disclosed are variants, having at least one substitution at position P2, P3, P4, P5, P6, or P7, wherein substitution at P2 can be P, wherein the substitution at P3 can be D, E, H, K, L, P, Q, R, W, or Y, wherein the substitution at P4 can be L, N, Q, V, or W.

Disclosed are variants, having at least one substitution at position P7 or P5, wherein the substitution at P7 can be G, V, L, F, S, T, N, Q, H, R, or, K, and wherein the substitution at P5 can be D, S, T, N, Q, H, R, K, V, or G.

Disclosed are variants, having at least one substitution at position P7 or P5, wherein the substitution at P7 can be E, Q, V, L, F, S, T, H, or E, and wherein the substitution at P5 can be E, F, G, P, D, S, T, N, Q, H, R, K, or V.

Disclosed are variants of antithrombin III, comprising a substitution at position P2, wherein the substitution at P2 is a P, along with at least one other substitution disclosed herein.

Disclosed are variants of antithrombin III, comprising a substitution at position P3, wherein the substitution at P3 is a D, E, H, K, L, P, Q, R, W, or Y.

Disclosed are variants of antithrombin III, comprising a substitution at position P4, wherein the substitution at P4 is a L, N, Q, V, or W, and when the substitution of W occurs with at least one other substitution disclosed herein.

Disclosed are variants of antithrombin III, comprising at least one substitution at either position P3 and P4, wherein the substitution at P3 is D, E, H, K, L, P, Q, R, W, or Y, and wherein the substitution at P4 is L, N, Q, V, or W, and at least one substitution at P2, P5, P6, P7, and P8, wherein the substitution at P2 is P, P5 is E, F, G, or P, wherein the substitution at P6 is E, G, L, or T, wherein the substitution at P7 is E or Q, and wherein the substitution at P8 is E.

Disclosed are variants of antithrombin III, comprising at least two substitutions at P3 D and P4, wherein the substitution at P3 is D, E, G, H, I, K, L, N, P, Q, R, S, W, or Y, and wherein the substitution at P4 is L, N, Q, V, or W.

Disclosed are variants of antithrombin III, comprising at least two substitutions at either position P3 and P4, wherein the substitution at P3 is D, E, H, K, L, P, Q, R, W, or Y, and wherein the substitution at P4 is A, F, G, L, N, P, Q, V, or W.

Disclosed are variants of antithrombin III, comprising a substitution at least two substitutions at P2, P3 and P4, wherein the substitution at P2 is P, wherein the substitution at P3 is D, E, G, H, I, K, L, N, P, Q, R, S, W, or Y, and wherein the substitution at P4 is A, F, G, L, N, P, Q, V, or W.

Disclosed are variants of antithrombin III, comprising a substitution at least one substitution at P2, P3 and P4, wherein the substitution at P2 is P, wherein the substitution at P3 is D, E, H, K, L, P, Q, R, S, W, or Y, and wherein the substitution at P4 is L, N, Q, V, or W.

Disclosed are variants of antithrombin III, comprising a substitution at least one substitution at P3 and P4, wherein the substitution at P3 is D, E, H, K, L, P, Q, R, S, W, or Y, and wherein the substitution at P4 is L, N, Q, V, or W.

Disclosed are variants of antithrombin III, wherein the variant antithrombin III has a combined activity greater than or equal to plasma ATIII in a coupled assay.

Disclosed are variants of antithrombin III, wherein the variant antithrombin III if has a combined activity greater than or equal to 2, 5, or 10, times the activity of plasma ATIII in a coupled assay.

Disclosed are variants of antithrombin III, wherein the variant antithrombin III has an increased protease resistance.

Also disclosed are variants in Olson, et al., (1997) Arch Biochem Biophys 341(2):212-21; Bjork, et al., (1992) Biochem J 286 (Pt 3):793-800; and Garone, et al., (1996) Biochemistry 35(27):8881-9, all of which are herein incorporated by reference at least for material related to variant ATIIIs and their sequence and structure.
7. Conditions
   a) Shear Rate Ranges

The disclosed compositions and methods are related to the shear rate that occurs at the surface of, for example, blood vessels, or tubing or medical devices through which body fluids flow. The shear rate is related to the geometry and the flow rate of the liquid flowing through the vessel, tube, or device. Shear rate is determined as disclosed herein. The disclosed methods in certain embodiments include conditions where there is no flow and/or where there are shear rates of at least 20 sec⁻¹, 40 sec⁻¹, 60 sec⁻¹, 80 sec⁻¹, 100 sec⁻¹, 200 sec⁻¹, 300 sec⁻¹, 400 sec⁻¹, 500 sec⁻¹, 600 sec⁻¹, 700 sec⁻¹, 800 sec⁻¹, 900 sec⁻¹, 1000 sec⁻¹, 1100 sec⁻¹, 1200 sec⁻¹, 1300 sec⁻¹, 1400 sec⁻¹, 1500 sec⁻¹, 1600 sec⁻¹, 1700 sec⁻, 1800 sec⁻¹, 1900 sec⁻¹, 2000 sec⁻¹, 2100 sec⁻¹, 2200 sec⁻¹, 2300 sec⁻¹, 2400 sec⁻¹, 2500 sec⁻¹, 2600 sec⁻¹, 2700 sec⁻¹, 2800 sec⁻¹, 2900 sec⁻¹, 3000 sec⁻¹, 3100 sec⁻¹, 3200 sec⁻¹, 3300 sec⁻¹, 3400 sec⁻¹, 3500 sec⁻¹, 3600 sec⁻¹, 3700 sec⁻¹, 3800 sec⁻¹, 3900 sec⁻¹, or 4000 sec⁻¹, 4100 sec⁻¹, 4200 sec⁻¹, 4300 sec⁻¹, 4400 sec⁻¹, 4500 sec⁻¹, 4600 sec⁻¹, 4700 sec⁻¹, 4800 sec⁻¹, 4900 sec⁻¹, 5000 sec⁻¹, 5100 sec⁻¹, 5200 sec⁻¹, 5300 sec⁻¹, 5400 sec⁻¹, 5500 sec⁻¹, 5600 sec⁻¹, 5700 sec⁻¹, 5800 sec⁻¹, 5900 sec⁻¹, 6000 sec⁻¹, 6100 sec⁻¹, 6200 sec⁻¹, 6300 sec⁻¹, 6400 sec⁻¹, 6500 sec⁻¹, 6600 sec⁻¹, 6700 sec⁻¹, 6800 sec⁻¹, 6900 sec⁻¹, 7000 sec⁻¹, 7100 sec⁻¹, 7200 sec⁻¹, 7300 sec⁻¹, 7400 sec⁻¹, 7500 sec⁻¹, 7600 sec⁻¹, 7700 sec⁻¹, 7800 sec⁻¹, 7900 sec⁻¹, or 8000 sec⁻¹8100 sec⁻¹, 8200 sec⁻¹, 8300 sec⁻¹, 8400 sec⁻¹, 8500 sec⁻¹, 8600 sec⁻¹, 8700 sec⁻¹, 8800 sec⁻¹, 8900 sec⁻¹, 9000 sec⁻¹, 9100 sec⁻¹, 9200 sec⁻¹, 9300 sec⁻¹, 9400 sec⁻¹, 9500 sec⁻¹, 9600 sec⁻¹, 9700 sec⁻¹, 9800 sec⁻¹, 9900 sec⁻¹, 10000 sec⁻¹, 10100 sec⁻¹, 10200 sec⁻¹, 10300 sec⁻¹, 10400 sec⁻¹, 10500 sec⁻¹, 10600 sec⁻¹, 10700 sec⁻¹, 10800 sec⁻¹, 10900 sec⁻¹, 11000 sec⁻¹, 11100 sec⁻¹, 11200 sec⁻¹, 11300 sec⁻¹, 11400 sec⁻¹, 11500 sec⁻¹, 11600 sec⁻¹, 11700 sec⁻¹, 11800 sec⁻¹, 11900 sec⁻¹, 12000 sec⁻¹, 12100 sec⁻¹, 12200 sec⁻¹, 12300 sec⁻¹, 12400 sec⁻¹, 12500 sec⁻¹, 12600 sec⁻¹, 12700 sec⁻¹, 12800 sec⁻¹, 12900 sec⁻¹, 13000 sec⁻¹, 13100 sec⁻¹, 13200 sec⁻¹, 13300 sec⁻¹, 13400 sec⁻¹, 13500 sec⁻¹, 13600 sec⁻¹, 13700 sec⁻¹, 13800 sec⁻¹, 13900 sec⁻¹, 14000 sec⁻¹, 14100 sec⁻¹, 14200 sec⁻¹, 14300 sec⁻¹, 14400 sec⁻¹, 14500 sec⁻¹, 14600 sec⁻¹, 14700 sec⁻¹, 14800 sec⁻¹, 14900 sec⁻¹, 15000 sec⁻¹, 15100 sec⁻¹, 15200 sec⁻¹, 15300 sec⁻¹, 15400 sec⁻¹, 15500 sec⁻¹, 15600 sec⁻¹, 15700 sec⁻¹, 15800 sec⁻¹, 15900 sec⁻¹, or 16000 sec⁻¹ are present.
b) Time of Exposure

Also disclosed are aspects of the methods and compositions where the conditions present include varying times of exposure. For example, disclosed are conditions where the compositions are added preloading or by bolus or are added by continuous infusion.
c) Infusion Location

In one aspect disclosed herein are methods of administration of ATIII, for example, ATIII with increased affinity for bound heparin and HSPG under low and high shear rate conditions as disclosed herein. In certain aspects, the infusion is administered immediately upstream of the area where ATIII surface loading is desired, such as a stent. It is shown herein that there is an improved effect of ATIII binding to a heparin or HSPG loaded surface when the ATIII is injected near, upstream of the desired ATIII binding location. For example, in certain aspects, administration of the ATIII, such as a high affinity ATIII, such as an ATIII having the properties of a beta-ATIII, as disclosed herein, through the catheter during or after an angioplasty procedure or through the catheter after the placing of the stent, just upstream of the stent or angioplasty site is a suitable mode of ATIII drug administration.
8. Types of Systems

The disclosed compositions and methods can be used in many different types of systems. For example, the disclosed compositions and methods can be used in a subject in vivo, where the system is the vasculature of the subject, such as the arterial and venous vessels. For example, the compositions and methods can be used in this way after or during an angioplasty procedure. The methods and compositions can also be used in other systems, including heart pumps, stents, vascular grafts and catheters. The ATIII compositions can be applied to the devices before, during or after placing them into the body.

Typically the systems are related in that they include heparin or HSPG attached to a solid surface, such as a vessel or a tube, or metal stent. Thus, systems that are coated with heparin are disclosed systems.
9. Materials Coated with Heparin

On the basis of experimental studies demonstrating rapid inactivation of surface-localized thrombin by ATIII bound to immobilized heparin, heparin coating of artificial surfaces is considered a promising approach for preventing further thrombin generation, and therefore thrombus formation, on medical devices (Blezer, et al., (1997) J Biomed Mater Res 37:108-13). Accordingly, biomaterial and medical device manufacturers have introduced, or are developing, products with heparin-coated surfaces. As indicated above, in theory heparin molecules attached to biomaterial surfaces will recruit endogenous ATIII from the blood, and the bound and activated ATIII will neutralize thrombin and fXa in the vicinity of the device surface, thereby preventing clotting and other pathologic enzymatic reactions and improving implant function and performance. Where information is available, heparin coatings do appear to provide incremental improvements in device function, but fall short of completely solving clotting, occlusion, and restenosis problems.

For example, approximately one third of patients implanted with Micromed DeBakey LVADs (left ventricular assist devices) developed low blood flow and increased power consumption rates indicative of intrapump thrombosis, and required emergency thrombolytic treatment to restore flow (Rothenburger, et al., (2002) Circulation 106 [suppl I], I-189-92). To address this problem, MicroMed produced and implanted at least 38 pumps in which the internal surfaces were coated with end-point attached heparin (CBAS™, Carmeda AB) to prevent pump thrombosis and thromboembolic problems (Goldstein, (2003) Circulation 108 (suppl. II), 272-7). However, it can be inferred from discontinuation of work on these heparin-coated pumps, that the approach probably did not substantially reduce thrombosis rates. As discussed above, the antithrombotic function of heparin coatings requires efficient transfer of endogenous ATIII from bulk, fluid phase blood to the device surface. While not wishing to be bound by theory, it is hypothesized that heparin-coated pump surfaces may not have bound enough ATIII because, as will be demonstrated in the Examples, the alpha ATIII isoform, accounting for ≧90% of the antithrombin in blood, binds poorly to heparin-coated surfaces at the high shear rates that are encountered in VADs.

Small-diameter vascular grafts are another category of implants that have received heparin-coatings. Autologous saphenous vein is the standard for peripheral and coronary artery bypass grafting, however, about 30% of patients do not have suitable veins due to vascular disease or previous harvesting for earlier bypasses. Even when available, recovery of autologous vessels is associated with extra surgical costs and morbidity, and 30 to 50% of these vein grafts become occluded by 10 years. Therefore, there is a real need for synthetic vascular grafts engineered to resist the development of thrombotic and proliferative occlusions. Synthetic grafts have been successful in applications where large diameter (>5-6 mm) conduits are implanted in areas of high blood flow. However, for smaller diameter, lower flow applications, patency is reduced due to thrombogenicity and anastomotic intimal hyperplasia. W. L. Gore and InterVascular manufacture heparin-coated vascular grafts for sale in Europe, but these products have not been approved by the FDA for USA use. Although heparin coating grafts modestly improved their patency rates in a canine carotid artery interposition model (Begovac, et al., (2003) Eur J Vasc and Endovasc Surg 25:432-7) and a prospective, double blind femoropopliteal bypass graft clinical trial (Devine, et al., (2001) J Vasc Surg 33:533-9), in both cases only about 50 to 60% of the heparin-coated implants remained open at the study endpoints (6 months and 3 years, respectively).

There are many different materials and devices that contain heparin and heparan sulfate proteoglycans which bear pentasacharide structures, can be placed on. One type of technology for placing heparin on surfaces is CBAS™ technology (Larm, et al., (1983) Biomaterials Med Devices Artif Organs 11:161, which is herein incorporated by reference at least for material related to heparin loading of surfaces and methods for performing the same). CBAS™ has been used to coat heparin onto surfaces of heart lung bypass machines (Cardiactech Inc.), stents, CBAS™ tubing, CBAS™ instech labs, amd heparin-coated extracorporeal circuits. Various devices such as blood oxygenator circuits for blood oxygenation have been coated with CBAS™ technology produced by, for example, Medtronic Inc. Ventricular assist devices have been coated and produced by, for example, Berlin Heart Inc. Vascular grafts have been coated and produced by, for example, Gore Inc. Coronary stents have been coated and produced by, for example, Cordis Inc. Central venous catheters have been coated and produced by, for example, CCL Inc. Intraarterial blood gas sensors have been coated and produced by, for example, Diametrics Inc. Continuous blood sampling catheters have been coated and produced by, for example, Carmeda Inc. Intraocular lens have been coated and produced by, for example, Pharmacia-Upjohn Inc.

CBAS™ can be used to coat heparin on thermoplastics, such as Delrin (DuPont), nylon, polycarbonate, polyethylene, polysulfone, polyurethane, PET, and PVC; rubbers, such as silicone and latex; metals, such as titantium, stainless steel, nitinol; wovens; and filter media, such as glass.

There are other technologies capable of coating heparin onto a surface. These technologies are discussed in Andersson, et al., (2003) J Bio Med Materials Res 67A (2):458, which is herein incorporated at least for material related to heparin coating and different means for performing the same. For example, there is a hepamed technique developed by Medtronics and the Corline Heparin Surface technique (vanDerGeissen (1999) Curr Interv Cardiology Rep 1:234, which is herein incorporated by reference at least for material related to heparin loading of surfaces and methods for performing the same.)

Thus, any device or material can have heparin coated on it, by any available means.

The disclosed relationship between binding vessel bound heparin and HSPG provides direction as to the concentration of how much heparin or HSPG to coat a device with. The relationship between the naturally occurring binding of the beta isoform of ATIII to a vessel bound heparin or HSPG rather than the alpha isoform is rooted in the differential binding affinities that each have for bound heparin or HSPG, which change under different shear rates.

There are many devices and materials that can have heparin or HSPG coated on them, and these devices and materials are typically brought into contact with the blood of a subject. Devices and materials that are brought into contact with the blood of a subject can be divided into two categories, 1) devices and materials which will be in continuous contact with the blood of the subject because they are being implanted in the subject and are intended for long term use, such as a coronary stent, and 2) devices and materials which are considered transient because the blood is simply circulating through the system for period of time, such as a heart lung machine. The discoveries disclosed herein, that there is an evolutionarily conserved relationship between the alpha and beta forms of ATIII and that this relationship has a very distinct purpose in partitioning one isoform of ATIII to vessel walls and one isoform to circulate in the free flowing blood, provides direction as to the type of heparin placement needed for a) systems that are approximating a blood circulatory system, such as a heart lung machine, and b) systems that are more long term, such as implants.

Thus, disclosed are devices with the concentrations of heparin and HSPG tailored as discussed herein to take advantage of the evolutionarily conserved relationship between the alpha and beta ATIII heparin binding, as well as methods of making these devices, and methods of using these devices using methods well known. For example, the devices can be made using CBAS™ technology.
10. Methods of Identifying Molecules that Bind Heparin/HSPGs with High Affinity in High Wall Shear Rate Conditions

Disclosed are methods for identifying super beta-ATIIIs that have enhanced affinity for vascular wall HSPG receptors and heparin-coated biomaterials, and that are resistant to inflammatory inactivation (wild type ATIII is extremely sensitive to cleavage and inactivation by neutrophil elastase). The structural basis of ATIII heparin binding and activation is discussed in for example, Olson, et al., (2002) Trends Cardiovasc Med 12:198-205; Jairajpuri, et al., (2002) J Biol Chem 277:24460-5; and Jairajpuri, et al., (2003) J Biol Chem 278:15941-50. In certain embodiments, the super beta-ATIIIs can have affinities greater than those published in Jairajpuri, et al., (2002) J Biol Chem 277:24460-5; Ersdal-Badju, et al., (1995) Biochem J 310:323-30, U.S. Pat. No. 5,420,252 (Kato); U.S. Pat. Nos. 5,618,713 and 5,700,663 (Zettlemeissl) high-heparin-affinity antithrombins.

Also disclosed are high-heparin-affinity elastase- and cathepsin G-resistant antithrombins for use in acute inflammatory environments (U.S. Patent Applications 60/085,197, 60/384,599, Ser. Nos. 09/305,588, 10/014,658, and PCT applications PCT/US99/10549 and PCT/US03/17506, which are herein incorporated by reference at least for material related to variant ATIIIs, including specific sequences of variant ATIII).

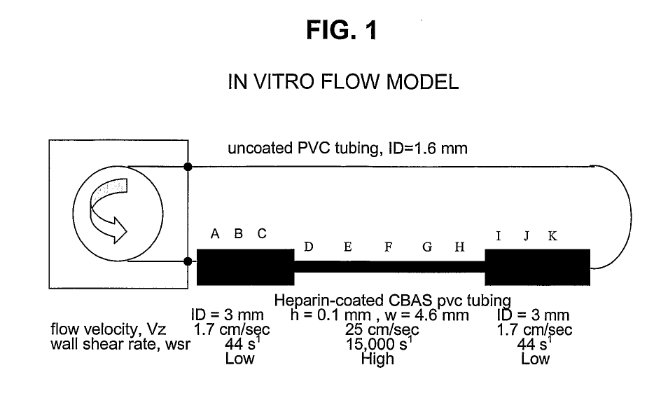

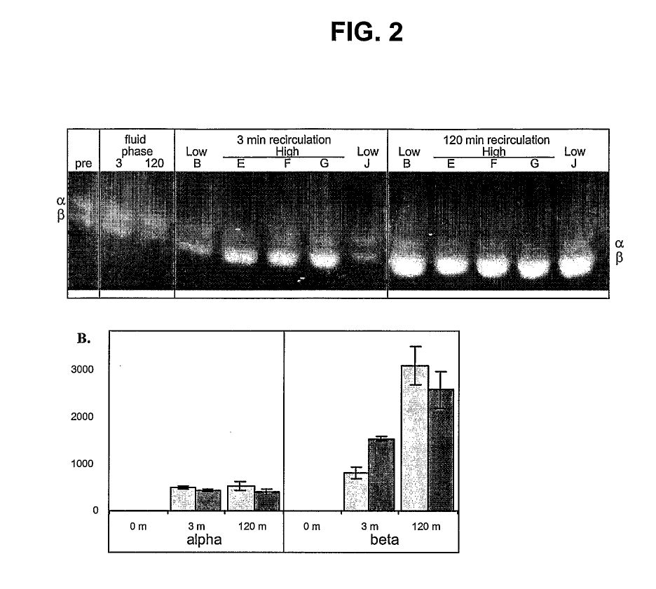

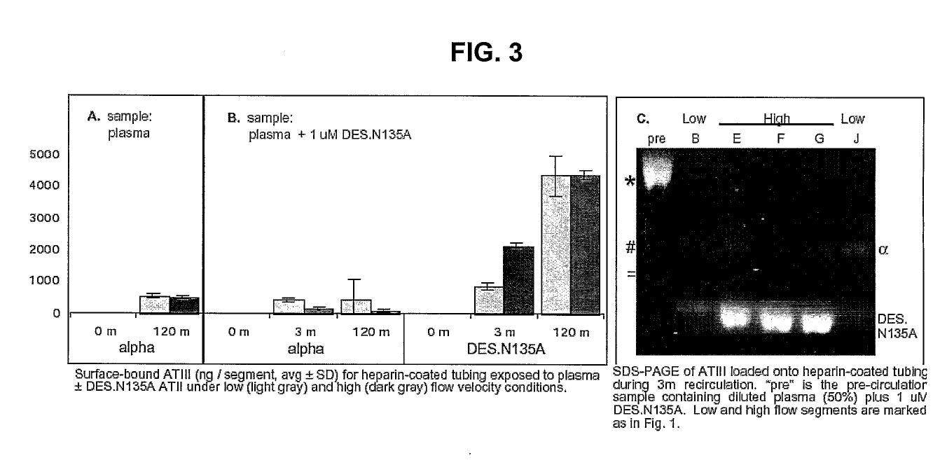

The disclosed methods take advantage of the information that it is preferred that ATIII bind heparin when it is bound to a vessel wall and/or under high shear conditions. The disclosed systems, such as the in vitro circulatory system disclosed in FIG. 1, can be used to screen various ATIII variants for activity. For example, a variant can be tested to determine whether it binds the high shear rate section of the tube, as is disclosed herein. Variants of ATIII can be made using any standard means of introducing variation into a sequence, such as discussed herein, for example, using PCR mutagenesis. It is understood that these mutations can be made on top of the mutations specifically already disclosed herein which, for example, increase heparin binding affinity or elastase or protease resistance. It is also understood that traditional binding assays and screening methods can also be employed to isolate ATIIIs or other molecules such as functional nucleic acids or monoclonal or polyclonal antibodies, which bind heparin in the way that ATIII binds heparin under low and high shear rate conditions.

It is understood that also disclosed are methods of making molecules that can be identified as described herein, by, for example, synthesizing the identified molecules. Also disclosed are the molecules which are so identified as well as methods of using these molecules.
11. Method of Coadministration with ATIII

Disclosed herein are methods of coadministration of ATIII with other compositions. For example, disclosed herein is a method of coadministering any of the ATIIIs disclosed herein with heparin in any form after, for example, a coronary angioplasty procedure or placement of a coronary stent. It is preferred that the ATIII, be high affinity ATIII, such as beta-ATIII or other ATIIIs as disclosed herein. In another example, any of the ATIIIs disclosed herein can be coadministered with low doses of a non-pentasaccharide containing anticoagulant, e.g., systemic anticoagulants such as direct thrombin or fXa inhibitors such as hirudin, argatroban, NAPc2, and the like.

C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular ATIII is disclosed and discussed and a number of modifications that can be made to a number of molecules including the ATIII are discussed, specifically contemplated is each and every combination and permutation of ATIII and the modifications that are possible unless specifically indicated to the contrary. Thus, if a class of molecules A, B, and C are disclosed as well as a class of molecules D, E, and F, and an example of a combination molecule A-D is disclosed, then even if each is not individually recited each is individually and collectively contemplated, meaning combinations A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Characteristics and Techniques for Biological Macromolecules Such as Proteins and Nucleic Acids

There are a number of properties and characteristics of biological macromolecules, such as sequence similarities, hybridizations, sequence variation, and so forth that are applicable to the disclosed ATIII and other molecules.
a) Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman, (1981) Adv Appl Math 2:482, by the homology alignment algorithm of Needleman and Wunsch, (1970) J Mol Biol 48:443, by the search for similarity method of Pearson and Lipman, (1988) Proc Natl Acad Sci USA 85:2444, by computerized implementations of these algorithms (GAP, BESTFIT, FASTA, and TFASTA in the Wisconsin Genetics Software Package, Genetics Computer Group, 575 Science Dr., Madison, Wis., or by inspection.

The same types of homology can be obtained for nucleic acids by for example the algorithms disclosed in Zuker, (1989) Science 244:48-52; Jaeger, et al., (1989) Proc Natl Acad Sci USA 86:7706-10; Jaeger, et al., (1989) Methods Enzymol 183:281-306, which are herein incorporated by reference for at least material related to nucleic acid alignment. It is understood to that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).
b) Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, to stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12° C. to 25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook, et al., Molecular Cloning: A Laboratory Manual, 2nd Ed., Cold Spring Harbor Laboratory, Cold Spring Harbor, N.Y., 1989; Kunkel, et al., (1987) Methods Enzymol 154:367, which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the limiting nucleic acid is bound to the non-limiting nucleic acid. Typically, the non-limiting primer is in, for example, 10 or 100 or 1000 fold excess. This type of assay can be performed under conditions where both the limiting and non-limiting primer are, for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly §0 it is a composition or method that is disclosed herein.
c) Nucleic Acids

There are a variety of molecules disclosed herein that are nucleic acid based, including for example the nucleic acids that encode, for example, ATIII as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.
(1) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger, et al., (1989) Proc Natl Acad Sci USA 86:6553-6),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH₂ or O) at the C6 position of purine nucleotides.
(2) Sequences

There are a variety of sequences related to, for example, ATIII, as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.
(3) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner.

Typically the disclosed primers hybridize with the nucleic acid or region of the nucleic acid or they hybridize with the complement of the nucleic acid or complement of a region of the nucleic acid.
d) Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BR-L, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan, et al., (1988) Proc Natl Acad Sci USA 85:4486; Miller, et al., (1986) Mol Cell Biol 6:2895). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani, et al., (1994) Hum Gene Ther 5:941-8), adeno-associated viral (AAV) vectors (Goodman, et al., (1994) Blood 84:1492-1500), lentiviral vectors (Naidini, et al., (1996) Science 272:263-7), pseudotyped retroviral vectors (Agrawal, et al., (1996) Exper Hematol 24:738-47). Physical transduction techniques can also be used, such as liposome delivery and receptor-mediated and other endocytosis mechanisms (see, for example, Schwartzenberger, et al., (1996) Blood 87:472-8). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, (1997) Hum Gene Ther 8:985-1001; Alvarez and Curiel, (1997) Hum Gene Ther 8:597-613). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.
e) Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.
(1) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers, et al., (1978) Nature, 273:113). The immediate early promoter of the human cytomegalovirus is conveniently obtained as a HindIII E restriction fragment (Greenway, et al., (1982) Gene 18:355-60). Of course, promoters from the host cell or related species also are useful herein.

Enhancer generally refers to a sequence of DNA that functions at no fixed, distance from the transcription start site and can be either 5′ (Laimins, et al., (1981) Proc Natl Acad Sci 78:993) or 3′ (Lusky, et al., (1983) Mol Cell Biol 3:1108) to the transcription unit. Furthermore, enhancers can be within an intron (Banerji, et al., (1983) Cell 33:729) as well as within the coding sequence itself (Osborne, et al., (1984) Mol Cell Biol 4:1293). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, fetoprotein, and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.
(2) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern and Berg, (1982) J Molec Appl Genet 1:327), mycophenolic acid, (Mulligan and Berg, (1980) Science 209:1422) or hygromycin, (Sugden, et al., (1985) Mol Cell Biol 5:410-13). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.
f) Peptides
   (1) Protein Variants

As discussed herein there are numerous variants of the ATIII protein that are known and herein contemplated. In addition, to the known functional ATIII strain variants there are derivatives of the ATIII proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 2 and 3 and are referred to as conservative substitutions.

TABLE 2
Amino Acid Abbreviations

| Amino Acid | Abbreviations |
|---|---|
| alanine | Ala (A) |
| alloisoleucine | AIle |
| arginine | Arg (R) |
| asparagine | Asn (N) |
| aspartic acid | Asp (D) |
| cysteine | Cys (C) |
| glutamic acid | Glu (E) |
| glutamine | Gln (Q) |
| glycine | Gly (G) |
| histidine | His (H) |
| isolelucine | Ile (I) |
| leucine | Leu (L) |
| lysine | Lys (K) |
| phenylalanine | Phe (F) |
| proline | Pro (P) |
| pyroglutamic acid | pGlu |
| serine | Ser (S) |
| threonine | Thr (T) |
| tyrosine | Tyr(Y) |
| tryptophan | Trp (W) |
| valine | Val (V) |

TABLE 3
Amino Acid Substitutions
Original Residue Exemplary Conservative
Substitutions, others are known in the art.

Ala [symbol] ser
Arg [symbol] lys or gln
Asn [symbol] gln or his
Asp [symbol] glu
Cys [symbol] ser
Gln [symbol] asn or lys
Glu [symbol] asp
Gly [symbol] pro
His [symbol] asn or gln
Ile [symbol] leu or val
Leu [symbol] ile or val
Lys [symbol] arg or gln;
Met [symbol] Leu or ile
Phemet [symbol] leu or tyr
Ser [symbol] thr
Thr [symbol] ser
Trp [symbol] tyr
Tyr [symbol] trp or phe
Val [symbol] ile or leu

Substantial changes in function or immunological identity are made by selecting substitutions that are less conservative than those in Table 3, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, IIe, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g., Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 (1983)), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 1 sets forth a particular sequence of ATIII. Specifically disclosed are variants of these and other proteins herein disclosed which have at least 70%, 75%, 80%, 85%, 90%, or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman, (1981) Adv Appl Math 2:482, by the homology alignment algorithm of Needleman and Wunsch, (1970) J Mol Biol 48: 443, by the search for similarity method of Pearson and Lipman, (1988) Proc Natl Acad Sci USA. 85:2444, by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, (1989) Science 244:48-52; Jaeger, et al., (1989) Proc Natl Acad Sci USA 86:7706-10; Jaeger, et al., (1989) Methods Enzymol 183:281-306, which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e., all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:1 is set forth in SEQ ID NO:2. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 2 and Table 3. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson, et al., (1991) Methods in Molec Biol 77:43-73; Zoller, (1992) Current Opinion in Biotechnology, 3:348-54; Ibba, (1995) Biotechnology &Genetic Engineering Reviews 13:197-216; Cahill, et al., (1989) TIBS 14(10):400-3; Benner, (1994) TIB Tech 12:158-63; Ibba and Hennecke, (1994) Biotechnology 12:678-82, all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—. These and others can be found in Spatola, A. F., in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, (1980) Trends Pharm Sci pp. 463-68; Hudson, et al., (1979) Int J Pept Prot Res 14:177-85 (—CH₂NH—, CH₂CH₂—); Spatola, et al., (1986) Life Sci 38:1243-9 (—CH H₂—S); Hann, (1982) J Chem Soc Perkin Trans I 307-14 (—CH—CH—, cis and trans); Almquist, et al., (1980) J Med Chem 23:1392-8 (—COCH₂—); Jennings-White, et al., (1982) Tetrahedron Lett 23:2533 (—COCH₂—); Szelke, et al., European Appln, EP 45665 CA (1982): 97:39405 (1982) (—CH(OH)CH₂—); Holladay, et al., (1983) Tetrahedron Lett 24:4401-4 (—C(OH)CH₂—); and Hruby, (1982) Life Sci 31:189-99 (—CH₂—S—); each of which is incorporated herein by reference. A particularly preferred non-peptide linkage is —CH₂NH—. It is understood that peptide analogs can have more than one atom between the bond atoms, such as beta-alanine, γ-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch, (1992) Ann Rev Biochem 61:387, incorporated herein by reference).
g) Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the disorder being treated and the risk of thrombosis/restenosis, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., (1991) Bioconjugate Chem 2:447-51; Bagshawe, (1989) Br J Cancer 60:275-81; Bagshawe, et al., (1988) Br J Cancer 58:700-3; Senter, et al., (1993) Bioconjugate Chem 4:3-9; Battelli, et al., (1992) Cancer Immunol Immunother 35:421-5; Pietersz and McKenzie, (1992) Immunolog Reviews 129:57-80; and Roffler, et al., (1991) Biochem Pharmacol 42:2062-5). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes, et al., (1989) Cancer Research 49:6214-20; and Litzinger and Huang, (1992) Biochimica et Biophysica Acta, 1104:179-87). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, (1991) DNA and Cell Biology 10(6):399-409).
(1) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflamniatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.
(2) Therapeutic Uses

Effective dosages and schedules for administering the compositions may be determined empirically, and making such determinations is within the skill in the art. The dosage ranges for the administration of the compositions are those large enough to produce the desired effect in which the symptoms or disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith, et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.
h) Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.
i) Computer Readable Mediums

It is understood that the disclosed nucleic acids and proteins can be represented as a sequence consisting of the nucleotides or amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums on which the nucleic acids or protein sequences can be recorded, stored, or saved.

Disclosed are computer readable mediums comprising the sequences and information regarding the sequences set forth herein.
j) Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

D. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta, et al., (1984) Ann Rev Biochem 53:323-56, (phosphotriester and phosphite-triester methods), and Narang, et al., (1980) Methods Enzymol 65:610-20, (phosphotriester method). Protein nucleic acid molecules can be made using known methods such as those described by Nielsen, et al., (1994) Bioconjug Chem 5:3-7.

2. Peptide Synthesis

One method of producing the disclosed proteins is to link two or more peptides or polypeptides together by protein chemistry techniques. For example, peptides or polypeptides can be chemically synthesized using currently available laboratory equipment using either Frnoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY, which are herein incorporated by reference at least for material related to peptide synthesis).

Alternatively, the peptide or polypeptide is independently synthesized in vivo as described herein. For example, the proteins, peptides, and polypeptides, can be produced in systems which produce fully and appropriately glycosylated versions of the ATIII. For example, advances in recombinant glycoprotein production methods, which allow more cost effective production of human glycoproteins by colonies of transgenic rabbits (www.bioprotein.com) or by yeast strains carrying human N-glycosylation system enzymes (Hamilton, et al., (2003) Science 301:1244-6; Gerngross, (2004) Nature Biotechnology 22:1409) can be used.

Once isolated, independent peptides or polypeptides may be linked, if needed, to form a peptide or fragment thereof via similar peptide condensation reactions. For example, enzymatic ligation of cloned or synthetic peptide segments allow relatively short peptide fragments to be joined to produce larger peptide fragments, polypeptides or whole protein domains (Abrahmsen, et al., (1991) Biochemistry 30:4151). Alternatively, native chemical ligation of synthetic peptides can be utilized to synthetically construct large peptides or D polypeptides from shorter peptide fragments. This method consists of a two step chemical reaction (Dawson, et al., (1994) Synthesis of Proteins by Native Chemical Ligation. Science 266:776-9). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini, et al., (1992) FEBS Lett 307:97-101; Clark-Lewis, et al., (1994) J Biol Chem 269:16075; Clark-Lewis, et al., (1991) Biochemistry 30:3128; Rajarathnam, et al., (1994) Biochemistry 33:6623-30).

Alternatively, unprotected peptide segments are chemically linked where the bond formed between the peptide segments as a result of the chemical ligation is an unnatural (non-peptide) bond (Schnolzer, et al., (1992) Science 256:221). This technique has been used to synthesize analogs of protein domains as well as large amounts of relatively pure proteins with full biological activity (deLisle Milton, et al., (1992) Techniques in Protein Chemistry IV. Academic Press, N.Y., pp. 257-67).

### VI. EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### A. Example 1

### Flow Effects on Loading of Endogenous ATIII Isoforms onto Heparin-Coated Surfaces

To investigate the contribution of flow to antithrombin III isoform partitioning between the blood and vascular surfaces, a model flow system was built from a peristaltic pump and pieces of uncoated and heparin-coated polyvinyl chloride (PVC) tubing (FIG. 1). The Carmeda BioActive Surface (CBAS™) 3 mm ID (internal diameter) endpoint-attached heparin-coated tubing (Larm, et al., (1983) Biomater Med Dev Artif Organs 11: 161-3) was kindly provided by Dr. Johan Reisenfeld, Carmeda AB, Stockholm, Sweden. The middle section of the heparin-coated tubing was compressed to generate a gap height of 0.1 mm and width of 4.6 mm. This manipulation allowed ATIII binding to the surface at both low and high flow velocity and wall shear rates to be compared in the same experiment.

FIG. 2 shows an experiment in which the sample was 4 mL of a solution containing 1 μM each purified human plasma ATIII isoforms, as illustrated in the “pre” lane at the left edge of the gel. This sample was recirculated through the flow system for 3 or 120 min at a volumetric flow rate of Q=7 mL/min. This led to a flow velocity of 1.7 cm/sec and a wall shear rate of 44 sec⁻¹ in the “low flow” uncompressed tubing segments, and of 25 cm/sec and 15,000 sec⁻¹ in “high flow” compressed section. Following recirculation of the sample for the indicated duration, circuits were washed with 20 mL 0.05 M Tris-HCl—NaCl, pH 7.4, 0.15 I. Then, the heparin-coated tubing was cut into 11 pieces of equal length. (A through K) Surface-bound protein was eluted from lumenal walls of individual pieces with gel buffer (8% SDS, 10% beta-mercaptoethanol (γ-ME), 0.25M Tris, pH 6.8). Bound proteins were resolved on 9% polyacrylamide Laemmli gels stained with Sypro Red. Binding of ATIII to surfaces in this model was heparin coating dependent; there was no binding to circuit sections made from uncoated PVC tubing.

After 3 minutes of recirculation, (5 passes of the sample through the circuit), heparin-coated tubing segments that had been exposed to low flow (B and J lanes in FIG. 2A, and light gray bars in FIG. 2B) bound a total of about 1300 ng ATIII/segment. The isoform ratio of the surface-bound ATIII was about 60:40 beta:alpha, versus the 50:50 ratio of the “pre” sample. Total surface-bound ATIII for the low flow tubing segments increased by about 3 fold to about 3600 ng per segment at 120 min (210 passes of the sample through the circuit), due to a net increase in beta ATIII, but not alpha ATIII, binding.

For the compressed, high flow, heparin-coated segment of the circuit (E, F, G lanes in FIG. 2A, and dark gray bars in FIG. 2B), total bound ATIII was about 2000 ng at 3 minutes, and about 3000 ng at 120 min. The loading rate of beta ATIII onto surfaces in high flow regions was significantly faster than its loading rate onto surfaces in low flow regions. Surfaces exposed to the sample for 3 minutes at high flow and high wall shear rates bound twice as much beta as surfaces exposed in the same experiment to low flow and low wall shear rates, while the alpha amounts on both surfaces were similar.

The left lanes of the gel show that differential binding of alpha and beta ATIII to the heparin-coated surface led to reciprocal changes in isoform composition of the circulating fluid phase. The isoform ratio in the fluid phase after 3 minutes exhibited a slight increase in alpha isoform content, and by 120 minutes, alpha dominated in the fluid phase due to significant depletion of beta ATIII by way of surface binding.

The results of the experiment shown in FIG. 2 provide an explanation for the low levels of beta ATIII in blood. Further, mass transport and the affinity of the ATIII-pentasaccharide binding interaction influence the efficiency of antithrombin surface loading. Thus, better control of blood-biomaterial and vascular surface interfacial coagulation, signaling, and proliferative pathways can be achieved by administration of the ATIII beta isoform, or recombinant ATIIIs with enhanced affinity for heparin.

1. Low Levels Of Beta ATIII in Blood

Example 1 on the partitioning of purified human plasma-derived ATIII isoforms between a flowing fluid phase and a heparin-coated biomaterial surface can be viewed as a model for the partitioning of endogenous ATIII isoforms between the blood and HSPG-bearing vessel surfaces of the circulatory system. Radio-labeled ATIII clearance studies in rabbits and humans suggest there are three pools of ATIII in the body: a plasma pool containing about 40%, a non-circulating vascular associated pool containing about 10%, and an extravascular pool containing about 50% of the total ATIII (Carlson, et al., (1984) J Clin Invest 74:191-9; Carlson, et al., (1985) Blood 66:13-9). The ATIII isoform content of circulating blood is about 90% alpha and about 10% beta, whereas extracts of vessel wall from normal and injured rabbit aorta (Witmer and Hatton, (1991) Arteriosclerosis and Thrombosis 11:530-9) and human saphenous vein (Frebelius, et al., Thrombosis and Haemostasis 78:433A) were enriched in beta-ATIII content relative to plasma. Against this background, the observed combination of alpha-ATIII predominance in blood and beta-ATIII enrichment on vascular surfaces is due to higher beta-ATIII affinity for the pentasaccharide sequence of heparin and HSPGs (Turk, et al., (1997) Biochemistry 36:6682-91) contributing to its more rapid loading onto vascular surfaces, particularly in regions of high wall shear rates. In contrast to flow-dependent changes in the beta-ATIII rate of loading onto the heparin-coated tubing surface, rates of alpha-ATIII loading varied little under flow conditions corresponding to venous to stenotic wall shear rates. This property of the alpha isoform can serve to insure that adequate concentrations of ATIII remain in the blood for scavenging and neutralization of activated clotting enzymes that have escaped into the bulk fluid phase. The survival and evolutionary advantages associated with isoform specific control of ATIII target enzymes in blood and vessel wall compartments according to the above mechanism can explain why the partial glycosylation signal encoding the production of two ATIII isoforms is highly conserved (Picard, et al., (1995) Biochemistry 34:8433-40; Backovic and Gettins, (2002) J Proteome Res 1:367-73).

2. Mass Transport and ATIII Isoform-Pentasaccharide Binding Affinities Influence the Efficiency of Surface Loading

This work indicates that flow-related and binding affinity parameters greatly influence the loading of the endogenous ATIII isoforms onto heparin-coated surfaces. FIG. 2B data considered in conjunction with fluid dynamic principles show that alpha isoform surface loading is “reaction controlled.” The alpha loading rate did not increase as flow increased, implying that its relatively high K_{d} (lower affinity) for pentasaccharide cannot support efficient loading, even when increased numbers of alpha molecules are delivered to surface heparin receptors at high flow velocities. In contrast, beta ATIII was more efficiently loaded onto the surface at the higher flow rate, suggesting that its interaction with the surface is “transport controlled.” In this case, the lower K_{d} (higher affinity) of beta ATIII for pentasaccharide allows it's efficient binding to heparin surface receptors once it enters the diffusion boundary layer.

3. Beta-ATIII Isoform Administration for Improved Control of Coagulation, Signaling, and Proliferative Reactions Occurring at Blood-Biomaterial and Vascular Surface Interfaces

As discussed above, the beta ATIII concentration of plasma is already “depleted” to <10% under normal conditions, and probably drops even lower in the vicinity of thrombogenic sites where thrombin and fXa are present and increase ATIII consumption. Example 1 indicates that beta-ATIII supplementation can facilitate a faster rate of antithrombin surface loading and more rapid restoration of thrombin and fXa inhibitory activity at thrombogenic sites and can thereby promote better control over coagulation, signaling, and proliferative pathways. Adjunctive purified beta-ATIII can be advantageous over plasma-derived ATIII (primarily comprised of alpha-ATIII) for rapid surface loading of thrombin and fXa inhibitory activity at low and high blood flow regions, however, its benefit would be greater at higher flow rates. The superior loading properties of the endogenous beta isoform relative to the endogenous alpha isoform correlates with a 5-fold increase in its affinity for pentasaccharide. This relationship indicates that even more efficient loading of inhibitory activities onto heparin-coated biomaterials and actively thrombogenic vascular surfaces can be achieved using recombinant ATIIIs with further enhanced pentasaccharide affinity. Example 2 confirms this and demonstrates that increasing heparin affinity beyond that of the endogenous beta isoform produces additional improvements in ATIII loading efficiency.

### B. Example 2

### Highly Efficient Loading of Recombinant DES.N135A ATIII onto Heparin-Coated Surfaces

DES.N135A is a recombinant human ATIII variant in which the asparagine-135 N-glycosylation site was mutated to force production of a beta-like isoform. DES.N135A is expressed using a Drosophia S2 insect cell line, which modifies the three remaining intact N-glycosylation sites with complex oligosaccharides that are smaller than those attached by mammals. Like bv.N135A ATIII expressed in Lepidopteran insect cells (Ersdal-Badju, et al., (1995) Biochem J 310:323-30), DES.N135A binds the pentasaccharide sequence of heparin and HSPGs with 50× higher affinity than the major human plasma-derived alpha isoform, and with 10× higher affinity than the minor plasma-derived beta-ATIII isoform (Turk, et al., (1997) Biochemistry 36:6682-91; and M. Jairajpuri and S. C. Bock, unpublished). The enhanced affinity is due to an increase in the kₒₙ and a decrease in the k_{off} for the binding reaction. In addition to enhancing the pentasaccharide affinity of the recombinant beta-ATIII, the smaller N-linked glycans are responsible for an electrophoretic mobility shift, and DES.N135A runs faster than human plasma-derived alpha and beta-ATIIIs on Laemmli gels (see FIG. 3C).

Example 2 used the in vitro flow system of FIG. 1 to examine flow-dependent binding of ATIII for three different samples. First, diluted plasma (50%) was recirculated through the system at Q=7 mL/min for 120 min (FIG. 3A). Samples of diluted plasma (50%) plus 1 μM DES.N135A were also recirculated through the system for 3 (FIGS. 2B, C) or 120 (FIG. 2B) minutes, also at 7 mL/minute. Plasma (rather than the purified ATIII isoforms of Example 1) was chosen for this work in order to approximate more realistically physiological conditions representative of the proposed therapeutic applications. Plasma contains >200 different proteins, including ATIII at about 4 μM. As previously noted, plasma ATIII contains about 90% alpha and about 10% beta ATIII. So the concentrations of the isoforms in 50% diluted plasma are about 90 μg/mL alpha and about 10 μg/mL beta. At these concentrations, neither is visible by staining in the “pre” sample of the FIG. 3C gel. Only the most abundant plasma proteins (*, albumin; #, antitrypsin and Ig light chains; =, haptoglobin β chain) show up in the photograph.

FIG. 3A shows that after 120 minutes recirculation of the plasma-only sample, surface binding of about 500 ng of alpha ATIII was observed for segments that had been exposed to both low (light gray) and high (dark gray) flow conditions. Supplementation of the plasma with 1 μM DES.N135A (FIG. 3B) approximately doubled the total amount of surface-bound ATIII on the low flow surfaces of the circuit at 3 minutes. This factor was even greater (about 4×) for high flow sections. At 120 minutes, high and low flow surfaces of the circuit exposed to plasma plus DES.N135A contained about 9× more ATIII per segment than circuits exposed to unsupplemented plasma. Thus, Example 2 indicates that DES.N135A-based recombinant ATIIIs can be useful at relatively low doses for loading anti-thrombotic, anti-signalling, and anti-proliferative ATIII proteinase inhibitor activity onto at-risk biomaterial and native vascular surfaces.

### C. Example 3

### Shear Rate Dependent Partitioning of Plasma Derived Antithrombin III Isoforms and High-Heparin-Affinity Recombinant ATIII Between Circulating Blood and Vascular Surfaces

As disclosed herein, the production of two forms of antithrombin III with different heparin/HSPG affinities has been evolutionarily conserved because it promotes favorable partitioning of ATIII between the blood and vascular surfaces in contact with the blood under physiological conditions of flow. This is supported by experiments disclosed herein (e.g., Examples 1-2) in which human plasma derived antithrombin isoforms and recombinant ATIII were pumped through heparin-coated CBAS™ tubing in an effort to model of blood flowing though the HSPG lined circulatory system.

As illustrated in FIG. 4, regions where wall-shear-rates approximated those in the arterial and venous compartments were generated by compressing one half of the length of the CBAS tubing and allowing the other half to remain uncompressed. For example, at a volumetric flow rate of 1.4 mL/min, solutions flowing over lumenal surfaces in the compressed and non-compressed sections of the tubing were exposed to wall-shear-rates of about 2000 sec⁻¹ and about 9 sec⁻¹, respectively. Samples containing various mixtures of plasma-derived alpha and beta-ATIII isoforms and DES.N135A recombinant ATIII, a prototypical super-beta-ATIII, were injected into the circuit and exposed to it for various periods of time, at various flow rates. Then, after washing with normal saline, the tubing was cut into segments corresponding to regions with different shear rates. Surface-bound ATIII was eluted from the tubing pieces with SDS beta-mercaptoethanol gel buffer, and ATIII isoform composition determined by SDS-PAGE.

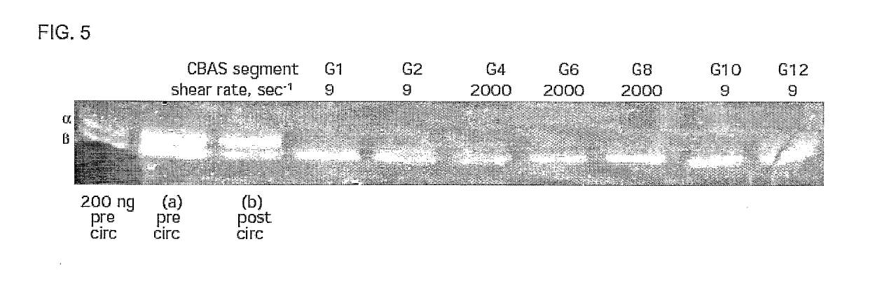

As discussed herein, the circulating ratio of ATIII isoforms is about 90 alpha to 10 beta, however several kinds of evidence suggests they are synthesized at a ratio of about 50:50. A solution composed of 50alpha:50beta was pumped through the FIG. 1 circuit for 4 hours. The total amount of ATIII in the recirculated sample was double the ATIII binding capacity of the CBAS™ tubing. At the end of the recirculation period, the ATIII solution was collected and the circuit washed with normal saline. The CBAS™ tubing from low and high wall-shear-rate sections of the circuit was cut into 2 cm segments, and bound ATIII eluted with SDS-P-ME. The isoform contents of pre- and postcirculation solutions and the CBAS™ tubing eluates were determined by 12% polyacrylamide gel electrophoresis as illustrated in FIG. 5.

FIG. 5 shows that after 4 hours of flow in the in vitro model of the circulatory system, the alpha:beta ratio of the “blood” shifted from 50alpha:50beta (lane a) to about 90alpha: 10beta (lane b), and that the heparin coated CBAS™ tubing representing the vascular endothelium bound mainly beta-ATIII. Also shown are the surface-bound ATIII results which show the isoform ratio of surface-bound ATIII eluted from CBAS™ segments #1-#12 exhibited increased content of the beta-ATIII isoform relative to alpha-ATIII isoform. Low shear rate segments (#1, #2, #10 and #12) appeared to bind more ATIII per unit of lumenal surface area than high shear rate segments (#4, #6 and #8) of the circuit.

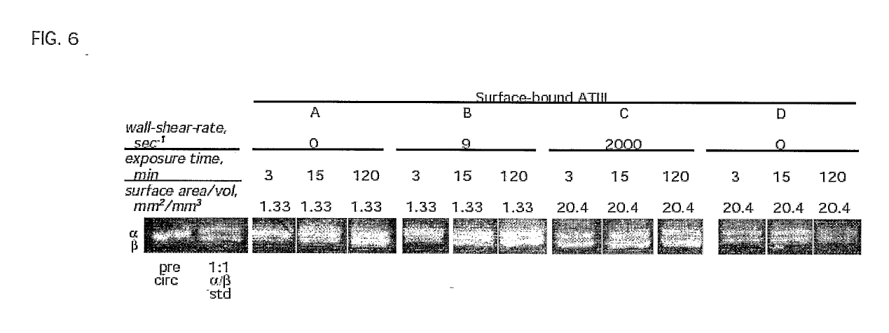

FIG. 6 shows differential binding of ATIII isoforms to the heparin-coated CBAS™ tubing as a function of exposure time under zero-flow, or low or high wall-shear-rate conditions. In this study, circuits were loaded with a sample that had an isoform ratio of >90alpha:<10beta, which is similar to plasma. The “pre” lane on the left side of FIG. 6 shows that beta-antithrombin is barely detectable in this sample. In contrast, significant amounts of beta-ATIII are observed on the tubing surface following exposure to the >90alpha:<10beta sample under various time, flow, and geometry conditions.

Under static, no flow conditions (FIG. 6: Panel A), the surface-bound alpha:beta ratio initially resembled that of the “pre” sample. However, with increased exposure time, the total amount of surfacebound ATIII and its beta:alpha ratio increased, consistent with the greater kₒₙ and reduced k_{off} that have been measured for the beta isoform under solution equilibrium binding conditions (Turk, et al., (1997) Biochemistry 36:6682-91) and progressive displacement of the alpha-isoform from heparin receptors on the wall.

The pattern of isoform and total antithrombin binding to the wall under low wall-shear-rate conditions (FIG. 6: Panel B) was qualitatively similar to the pattern obtained under obtained static conditions. Quantitatively, however, more antithrombin bound to the surface under low wall-shear-rate conditions due to recirculation-associated exposure of the heparin receptors to more molecules of ATIII as a result of mass transport.

The pattern of ATIII isoform surface binding in the high shear rate section of the circuit (FIG. 6: Panel C) is quite different from the patterns obtained under zero-flow and low shear rate conditions. Under high shear rate conditions, minimal alpha-ATIII bound to the surface, whereas beta-ATIII binding increased with time, resulting in a surface-bound ratio of about 80beta:20alpha after 2 hours. Apparently, a large differential in isoform binding affinities develops at high wall-shear-rates. This leads to reduced surface accumulation of alpha-ATIII and to stabile and cumulative binding of beta-ATIII.

Different patterns of isoform binding were observed in the non-compressed vs. compressed sections of the circuit under zero-flow conditions (see panels A versus D of FIG. 6, corresponding to segments 2 versus 6 of FIG. 4). These differences suggest that vessel diameters (which vary greatly through out the circulatory tree and lead to large variations in surface area to blood volume and diffusion distance values) also affect how antithrombin isoforms partition between the blood and the vessel wall.

Results from the FIGS. 5 and 6 experiments, and from FIGS. 1-2, suggest that the difference in alpha-ATIII and beta-ATIII binding affinities for heparin is magnified under flowing conditions, and that this leads to preferential binding of beta-ATIII on vascular surfaces and alpha-ATIII relegation to the blood, as is observed physiologically. Moreover, the experiment shown in FIG. 6 demonstrates that despite its low initial concentration in the circulating sample, beta-ATIII is still efficiently sequestered from the fluid phase to target and bind the heparin-coated surface. This observation indicates that “super” beta-antithrombin molecules with further increases in their heparin affinity can offer additional dosing advantages with respect to loading vascular and heparin-coated medical device surfaces with antithrombotic and anti-inflammatory ATIII. For the earliest 3 min time point of the static experiment, the isoform ratio of surface-bound ATIII from the uncompressed tubing segment #2 (A) resembled that of the input sample. However, as exposure time increased, less alpha-ATIII and more beta-ATIII were recovered. Therefore, under static conditions in the low (1.33) surface area-to-volume, low receptor-to-ligand segment of the circuit, ATIII isoforms initially bound the CBAS surface according to the law of mass action. With increased exposure time, alpha was displaced and beta-ATIII accumulated on the surface, consistent with beta's higher heparin affinity. Surface bound ATIII from compressed tubing segment #6 (D) exhibited a about 50alpha:50beta isoform ratio at all time points of the static, no-flow experiment. In this case, the high (20.4) surface area-to-volume ratio indicates that a reduced number of ligand molecules are available to the same number of receptors. Consequently, beta-ATIII saturation of the surface-bound ATIII profile appears to occur earlier in the time course, and significant alpha-ATIII displacement does not occur due to D the low beta-ATIII content in the fluid phase of the compressed segment.

To investigate this possibility, a solution containing plasma derived alpha-ATIII, plasma derived beta-ATIII and recombinant DES.N135A ATIII was circulated through the in vitro circulatory system model. The measured solution equilibrium binding K_{d}s of plasma alpha-ATIII, plasma beta-ATIII, and recombinant DES.N135A for high affinity heparin at pH 7.4 and 0.3 I are respectively 300 nM, 54 nM, and 6 nM (Turk, et al., (1997) Biochemistry 36:6682-91). This means that under zero-flow conditions, plasma beta-ATIII exhibits a 6-fold higher affinity for heparin than does plasma alpha-ATIII, and that recombinant DES.N135A ATIII has a 50-fold higher affinity for heparin than does plasma alpha-ATIII, and a 9-fold higher affinity for heparin than does plasma beta-ATIII.

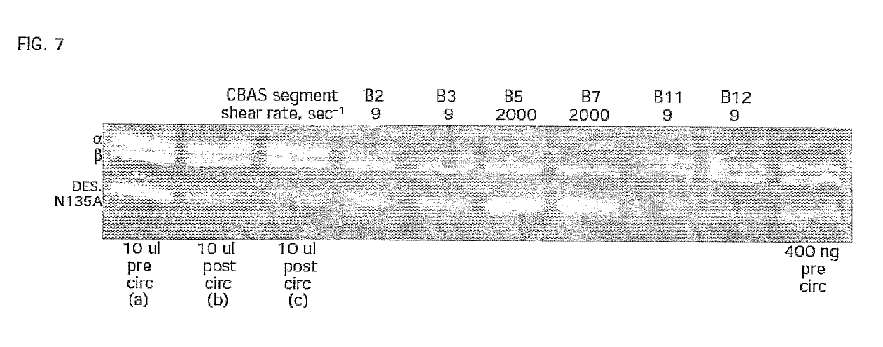

The left lanes of FIG. 7 show a sample initially containing alpha, beta, and DES.N135A antithrombins in an about 40:40:20 ratio before (lane a) and after (lanes b and c) 15 minutes of recirculation through the FIG. 4 circuit. The fluid phase was selectively depleted of the DES.N135A species that has the highest affinity for heparin due to its capture on surfaces of the proximal low wall-shear-rate and high wall-shear-rate sections of the circuit. The species with the next highest heparin affinity, plasma derived beta-antithrombin, bound to surfaces in low and high wall-shear-rate sections of the circuit. The lowest affinity ATIII-alpha isoform bound only to surfaces in low shear rate segments of the circuit. Collectively, these binding patterns indicate that as the wall-shear-rate increases, corresponding increases in ATIII heparin/HSPG binding affinities are required to mediate stabile binding to the surface. Conversely, high wall-shear-rates are non-permissive for binding of lower affinity antithrombins (e.g., alpha-ATIII) to the wall.

Additionally, binding of DES.N135A and beta-ATIII is inferred to occur very rapidly, based on the greater presence of these species on proximal versus distal low wall-shear-rate regions of the circuit. The increased binding of alpha-ATIII observed in distal versus proximal low wall-shear-rate regions of the circuit probably results from increased receptor availability due reduced DES.N135A and beta-ATIII binding in this region.

The experiments shown in FIGS. 5-7 indicate that beta-antithrombins with enhanced affinities for heparin/HSPG are useful at low doses for augmenting ATIII-mediated antithrombotic and anti-inflammatory activities on the vessel surface, especially in regions of the circulation having high wall-shear-rates.

Equilibrium binding measurements of antithrombin affinities for heparin conducted with both the ligand and receptor in solution have been used to establish the relative affinities of plasma ATIII isoforms and recombinant antithrombins for heparin. However, the disclosed studies with antithrombin isoforms flowing through a CBAS™ heparin-coated circuit indicate that several additional factors can be considered in order to properly describe the physiologically relevant interaction of antithrombin III binding to vascular wall heparan sulfate proteoglycans. Among these considerations are that (1) the HSPG receptors are immobilized on the vessel surface, rather than free in solution like pharmaceutical heparin, and that (2) due to variations in flow rates and vessel geometry in different parts of the circulatory tree, the binding reaction actually occurs under a wide range of wall-shear-rates.

In a semi-quantitative way, the 3 mm CBAS™ tubing studies show that high wall-shear-rates are less permissive for ATIII-alpha isoform binding to surface-bound heparin, and that with increasing shear rates, beta-ATIII is preferentially bound. These studies also reveal that due to its higher affinity, the beta-isoform can still effectively target the surface when its concentration in the circulating fluid phase is very low compared to that of the alpha-isoform that predominates in circulating blood. This indicates that at very low doses “super” beta-antithrombins with enhanced heparin affinity can effectively target ATIII antithrombotic and anti-inflammatory activity to the vessel wall.

Kinetic studies of alpha and beta-ATIII binding to a heparin-coated surface suggest that distinct, shear rate-dependent mechanisms influence the isoform composition of surface-bound ATIII in low vs. high shear rate regions of the vasculature. No-flow conditions and low wall shear rates are permissive for the binding of both ATIII isoforms, which are initially recovered from these surfaces in accordance with their proportions in the fluid phase. Then, beta-ATIII progressively displaces alpha-ATIII, due to presumably faster kₒₙ and slower k_{off} values associated with beta-ATIII's enhanced affinity for heparin. At higher wall shear rates, conditions are permissive for the binding of beta-ATIII, but become less permissive for alpha-ATIII binding. Beta-ATIII content on these high shear rate surfaces is therefore initially high and exhibits further increases with time.

This study demonstrates that rheological factors influence ATIII isoform interactions with heparin-coated surfaces. The work further indicates that the high alpha-ATIII content of the blood and the reciprocally high beta-ATIII content of vascular surfaces result from shear rate-dependent partitioning of the isoforms between the blood and vascular surfaces. It was also demonstrated that over a broad range of physiologically and pathologically relevant shear rates, beta-ATIII efficiently bound a heparin-coated surface, despite its initially trace concentration in the fluid phase. This finding suggests that low dose beta-ATIII and beta-ATIII derivatives are particularly well suited for augmenting the anticoagulant and antithrombotic properties of vascular surfaces and heparin-coated medical devices.

### D. Example 4

### Shear Rate-Dependent Partitioning of Antithrombin III Isoforms and Variants

Antithrombin III (ATIII) is a plasma proteinase inhibitor and essential endogenous anticoagulant molecule. ATIII inactivates coagulation enzymes such as thrombin and factor Xa (fXa). ATIII binding to pharmaceutical heparin and vessel wall heparan sulfate proteoglycan (HSPG) receptors accelerates its thrombin and fXa inhibition rates about 1000 fold, and targets its anticoagulant activity to the vascular surface compartment where these enzymes are generated.

There are two ATIII isoforms: alpha-ATIII and beta-ATIII. The alpha isoform predominates in circulating blood. Plasma contains about 90% alpha and about 10% beta ATIII. ATIII isoform production is evolutionarily conserved, suggesting that it is advantageous to carry both species, and that alpha and beta have distinct and critical functions. Beta-ATIII binds the heparin/HSPGs with 5× higher affinity than alpha ATIII. Beta ATIII interacts preferentially with the vascular endothelium. HSPG-bound ATIII contributes substantially to anticoagulant and antithrombotic properties of the endothelium.

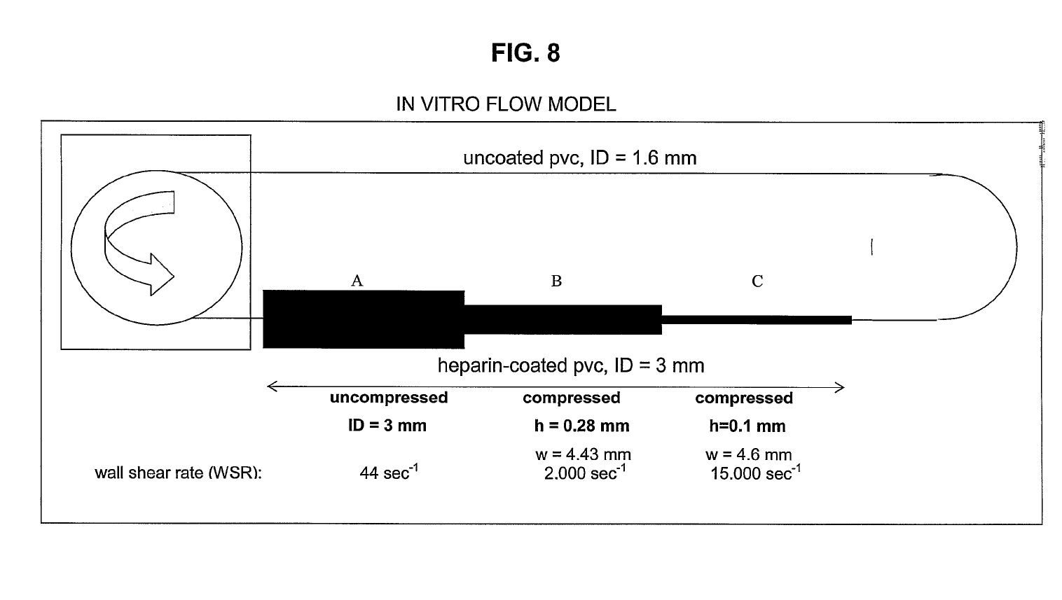

FIGS. 8-11 show the results of experiments with alpha, beta, and variant ATIIIs, such as N135A. The results of FIGS. 8-11 show that rheological factors influence ATIII binding to heparin-coated surfaces. Also, the beta-ATIII isoform binds to heparin-coated surfaces more rapidly than the alpha-ATIII isoform and the differential in loading efficiencies is greater a high wall shear rates. Furthermore, shear rate-dependent partitioning of the isoforms between the blood and vascular surfaces contributes to the high alpha-ATIII/low beta ATIII content of the blood and the reciprocally high beta-ATIII content of vascular surfaces. Results obtained for binding of ATIII isoforms and variants at a wide range of flow conditions indicate that (1) mass transport and (2) molecular binding affinity factors influence overall rates of ATIII loading onto heparin-coated biomaterial and HSPG-bearing vascular surfaces. Enhancing the heparin-binding affinity of a recombinant ATIII promoted more efficient loading onto a heparin-coated biomaterial surface and improved functional inhibition of flowing thrombin.

The results disclosed herein indicate recombinant ATIIIs with enhanced affinity for heparin can be useful for augmenting the anticoagulant, antithrombotic and anti-inflammatory properties of natural vascular surfaces and heparin-coated medical devices.

FIG. 8 shows an in vitro flow system constructed from 1.6 mm ID uncoated and 3.0 mm ID CBAS™ (Carmeda Bioactive Surface) heparin-coated PVC (polyvinylchloride) tubing. The flow rate was: Q=7 mL/min and wall shear rates were: 44 sec⁻¹ (venous) in section A; 2,000 sec⁻¹ (arterial) in section B; and 15,000 sec⁻¹ (pathological) in section B. Following recirculation of ATIII-containing samples through this system, the fluid phase (containing unbound ATIII) was collected and the circuit washed with buffered saline. CBAS™ tubing sections A, B, and C were each cut into three 2-cm segments. Surface-bound ATIII was eluted from the tubing pieces, and the isoform content determined by 10% SDS-PAGE.

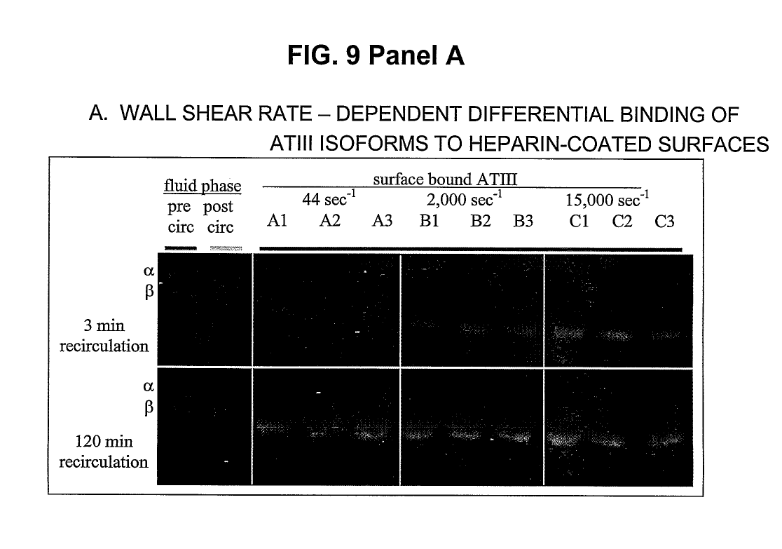

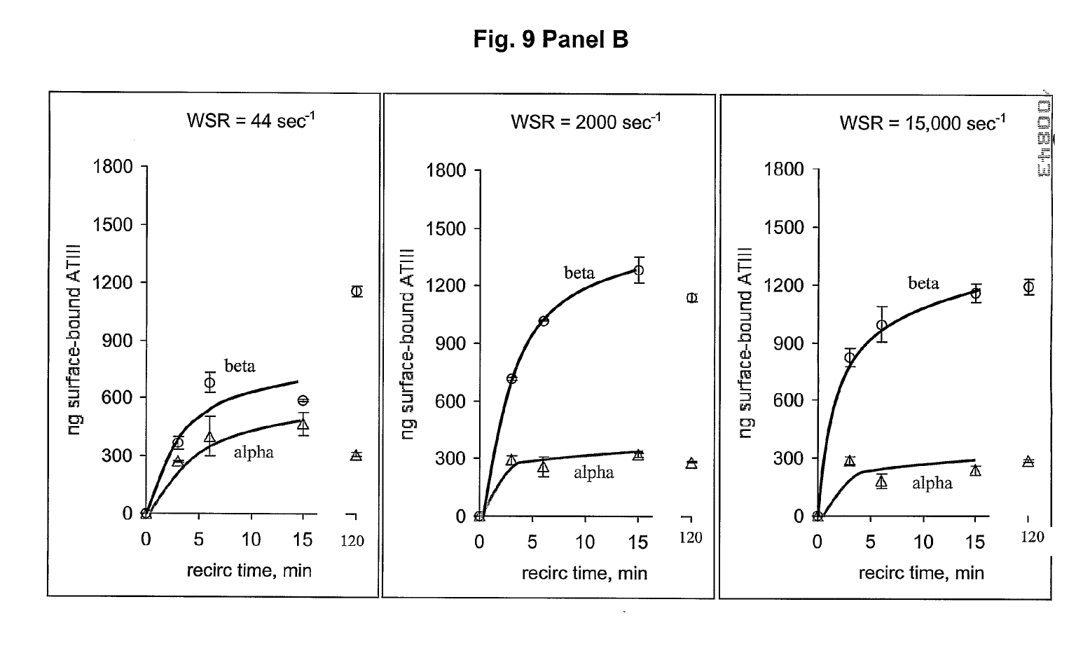

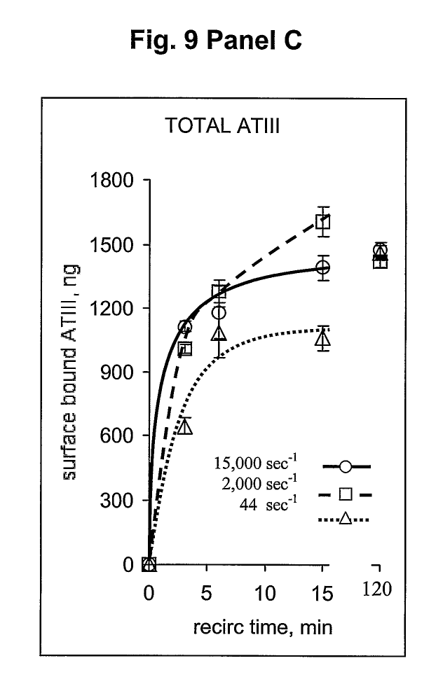

FIG. 9 shows the results of an experiment using the FIG. 8 model system under the above flow conditions and with 3, 6, 15, and 120 min exposures of samples containing 1 μM each of the human plasma-derived alpha and beta ATIII isoforms. The SDS-PAGE gel in FIG. 9A illustrates wall shear rate effects on ATIII isoform binding to the heparin-coated surface after 3 or 120 minutes of recirculation. The “fluid phase-pre-circ” lanes show the initial 1:1 alpha:beta ratio of the injected sample. The “surface-bound ATIII lanes” show more rapid 3 binding of beta-ATIII, especially at the higher wall shear rates encountered during arterial and pathological flow. Progressive depletion of beta-ATIII relative to alpha-ATIII is observed in 3 min and 120 min “fluid phase-post-circ” samples. FIG. 9B shows presents quantitative analysis of the experiment. At WSRs of 44 to 15,000 sec⁻¹, beta-ATIII bound to the heparin-coated surface more rapidly than alpha-ATIII. At the arterial and pathological WSRs, initial (3 min) rates of beta isoform loading were twice that at the venous WSR. In contrast, rates of alpha isoform loading were WSR-independent. FIG. 9C shows total ATIII bound as a function of time at different wall shear rates. Initial rates of ATIII binding to the heparin-coated biomaterial surface were faster in higher WSR sections of the circuit. At “equilibrium” (120 min), the amounts of surface-bound ATIII were similar for all wall shear rates.

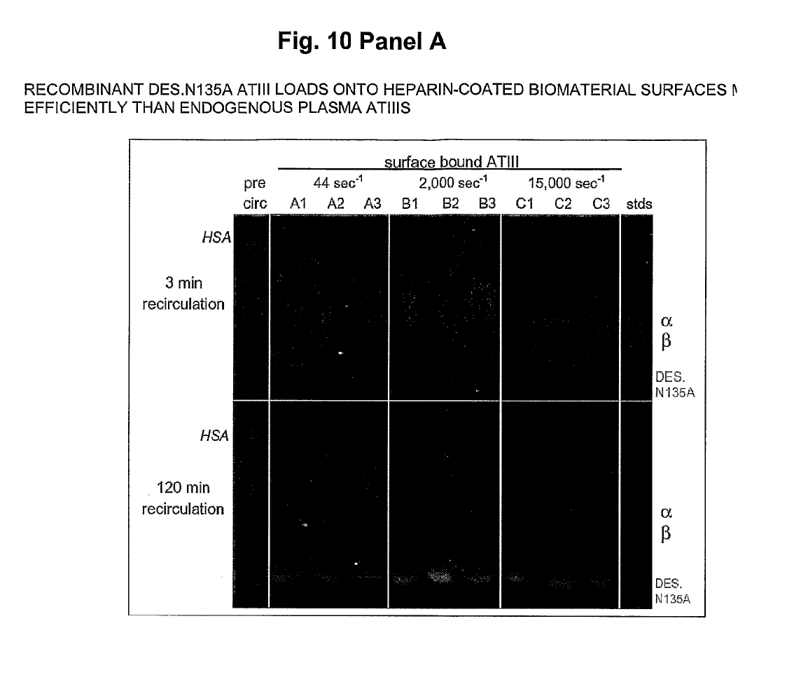

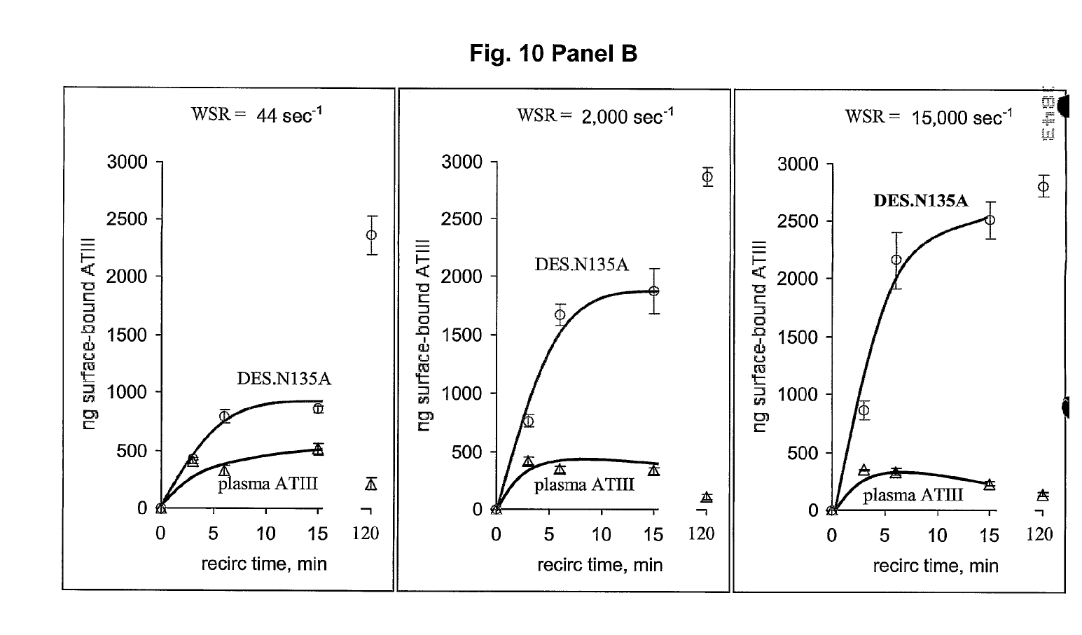

The FIG. 10 study used the same set-up and overall experimental design as the FIG. 9 study, but the sample was diluted human plasma (50%) supplemented with 1 μM recombinant DES.N135A ATIII. DES.N135A is a recombinant ATIII that binds heparin with 50 times higher affinity than alpha-ATIII (the major isoform in plasma), and with 10 times higher affinity than beta ATIII (the minor isoform in plasma). The diluted plasma (50%) contained about 1 μM ATIII of which about 90% was alpha isoform and about 10% was beta isoform. Plasma, rather than purified ATIII isoforms, was utilized in this experiment in order to model the clinical situation of super-beta ATIII administration to a patient. The FIG. 10A SDS-PAGE gels show that rate of DES.N135A surface loading exceeds the rate plasma ATIII (mostly alpha isoform) loading, and this difference was most notable at higher wall shear rates. At 3 “equilibrium” (120 min) under all flow conditions, most of the surface-bound ATIII was DES.N135A ATIII, rather than endogenous plasma-derived ATIII. FIG. 10B shows quantitative analysis of the data. The rate of plasma ATIII (mostly alpha ATIII) loading onto the heparin-coated biomaterial surface was largely independent of wall shear rate. Recombinant DES.N135A loaded onto the surface 2×, 5×, and 7× faster than plasma ATIII at wall shear rates of 44, 200, and 15,000 sec⁻¹, respectively. Under venous, arterial, and pathological flow conditions, supplementing plasma with 1 μM recombinant DES.N135A ATIII lead to >10-fold increases in the amount of surface bound ATIII at 120 minutes. Of particular interest is that the rate (i.e., efficiency) of DES.N135A surface loading still improved increasing from arterial flow conditions to pathological flow conditions. This contrasts with the behavior of the plasma beta ATIII isoform, which loaded at similar rates at the arterial and pathological wall shear rates (see FIG. 9B). Improved performance of the super-beta ATIII at pathological shear rates can be a valuable property for targeting anticoagulant and antithrombotic activity to thrombosis-prone stenotic and bifurcation regions, where there is elevated thrombin and fXa generation. The demonstrated in vitro behavior predicts that that super-beta ATIII will function more efficiently than plasma beta ATIII for replenishing antithrombin molecules on vacated heparin/HSPG receptors that develop as inhibitory complexes of ATIII with its target enzymes form and dissociate from the surface.

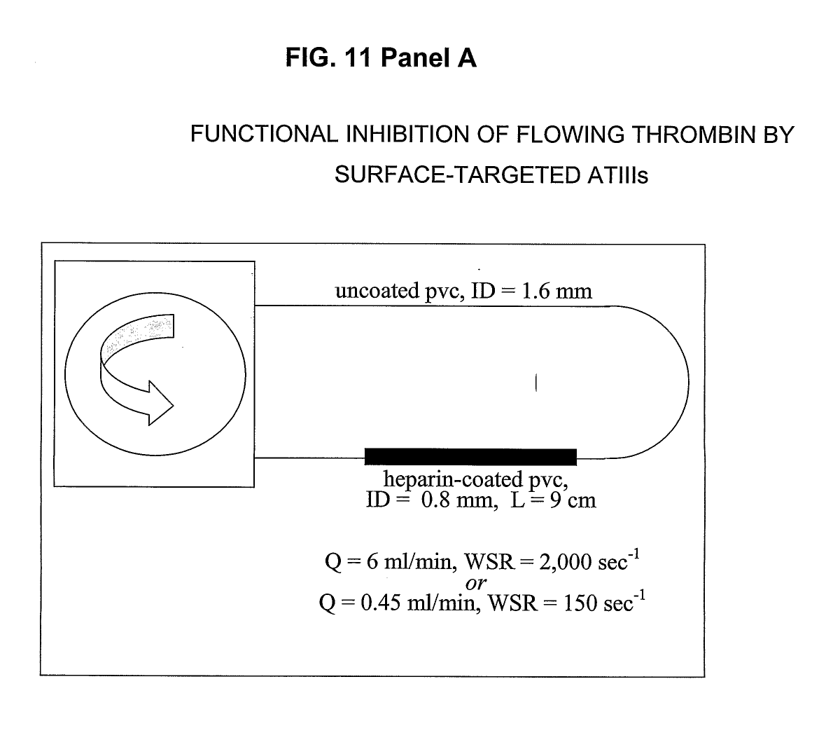

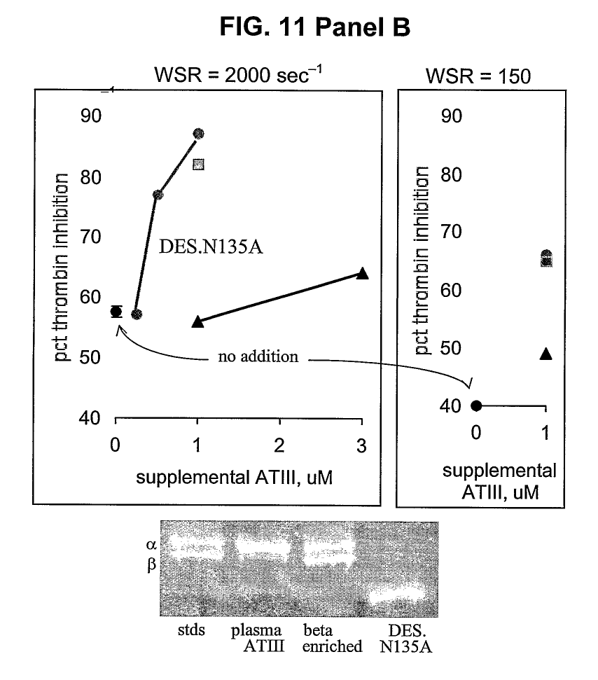

The FIG. 11 experiment compares functional inhibition of flowing thrombin by different ATIIIs loaded onto a heparin-coated surface under different flow conditions. Panel A shows the in vitro system used to measure inhibition of flowing thrombin by the surface-targeted ATIIIs this study. The experimental protocol involved the injection of 50% human plasma supplemented with saline (control) or ATIII, followed by recirculation for 15 min at flow rates producing wall shear rates of 150 or 2,000 sec⁻¹. Then the circuit washed with normal saline, and 10 nM human thrombin was injected and recirculated for 15 min. Finally, the fluid phase was recovered and residual thrombin activity measured by chromogenic assay. The FIG. 11B plots summarize data from this study, including “no addition” controls in which the circuit was exposed to unsupplemented 50% plasma at wall shear rates of 150 or 2,000 sec⁻¹, leading to basal 40% and 55% thrombin inhibition values, respectively. Exposure of the circuit to human plasma supplemented with up to 3 μM purified plasma-derived ATIII (triangles) [which is mostly alpha isoform (see gel lane 2)] produced a small increase in the levels of thrombin inhibition. Supplementation with lesser concentrations of beta-ATIII-enriched (squares) (see gel lane 3) or recombinant DES.N135A (round light grey dots) (see gel lane 4) produced more thrombin inhibition than supplementation with even greater concentrations of plasma-derived ATIII. Supplementation with 1 μM beta-enriched or DES.N135A ATIIIs produced 65% thrombin inhibition at the venous wall shear rate of 150 sec⁻¹, and 80% thrombin inhibition at the arterial wall shear rate of 2000 sec⁻¹. Thrombin inhibition by DES.N135A ATIII was dose dependent, and >85% for a 1 μM dose at the 2,000 sec⁻¹ wall shear rate. Thus, enhancing ATIII heparin-binding affinity increases anticoagulant surface loading and improves functional inhibition of flowing thrombin.

### E. Prophetic Example 5

### Parametric and Mechanistic Investigations of ATIII Isoform and Variant Binding to Heparin- and HSPG-Coated Surfaces Under Flow

Disclosed herein are methods of obtaining parametric and mechanistic information about endogenous ATIII isoform and recombinant ATIII variant binding to heparin-coated and HSPG-bearing surfaces. This information can be used to elucidate fluid dynamic and binding affinity contributions to ATIII surface loading, and to establish a solid understanding of factors that influence inhibitor delivery to and reaction with surfaces in different parts of the vascular tree and in different biomedical devices and cardiovascular pathologies.

### 1. Example 5a

### Loading of ATIII Molecules of Varying Heparin Affinities Onto Heparin-Coated Surfaces Under Steady Flow at Venous, Arterial and Pathological Wall Shear Rates

Examples 1-4 were conducted with a peristaltic pump that generated flow with poorly defined pulsatility, and using several different wall shear rates representative of venous, arterial, and pathological blood flow (9, 44, 2000, and 15,000 sec⁻¹). A larger, more systematic and better-controlled quantitative study can be performed in which endogenous ATIII isoforms and DES.N135A are exposed to a heparin-coated surface under steady flow at 4 different velocity and wall shear rate combinations. Data from this example can be used to identify what flow conditions are most favorable for surface loading of ATIIIs with different heparin affinities, and for evaluating a mathematical model of the mass transport and molecular binding affinity components of ATIII loading.

For this study, steady flow can be achieved by gravity. The 3 mm ID CBAS tubing used in the Examples 1-4 can be replaced with 20 cm long pieces of 0.8 mm ID CBAS heparin-coated tubing. Use of the narrower bore tubing can allow the production of the desired wall shear rates directly by simple variation of the flow rate. Table 4 summarizes the ATIII samples, flow conditions, and loading times that can be used in this study.

TABLE 4
Design of experiment to characterize ATIII isoform and variant
binding to a Heparin-coated surface under different conditions of steady flow

| | Molecular | Heparin K_{d}¹ | Wall shear rates; | |
|---|---|---|---|---|
| Samples | weight | pH 7.4, 0.3 I | Flow velocities² | Recirculation Times³ |
| alpha ATIII | 60,000 D | 300 nM | 100 sec⁻¹; 1 cm/sec | 3, 30, 120 min |
| | | | 332 sec⁻¹; 3.3 cm/sec | 3, 30, 120 min |
| | | | 2000 sec⁻¹; 20 cm/sec | 3, 30, 120 min |
| | | | 5000 sec⁻¹; 50 cm/sec | 3, 30, 120 min |
| beta ATIII | 57,000 D | 54 nM | 100 sec⁻¹; 1 cm/sec | 2, 4, 8, 16, 32 min |
| | | | 332 sec⁻¹; 3.3 cm/sec | 2, 4, 8, 16, 32 min |
| | | | 2000 sec⁻¹; 20 cm/sec | 2, 4, 8, 16, 32 min |
| | | | 5000 sec⁻¹; 50 cm/sec | 2, 4, 8, 16, 32 min |
| recombinant | 50,000 D | 6 nM | 100 sec⁻¹; 1 cm/sec | 2, 4, 8, 16, 32 min |
| DES.N135A ATIII | | | 332 sec⁻¹; 3.3 cm/sec | 2, 4, 8, 16, 32 min |
| | | | 2000 sec⁻¹; 20 cm/sec | 2, 4, 8, 16, 32 min |
| | | | 5000 sec⁻¹; 50 cm/sec | 2, 4, 8, 16, 32 min |

¹Turk, et al., (1997) Biochemistry 36: 6682-91, from equilibrium binding measurements under static conditions.
²Using 0.8 mm ID tubing, the listed wall shear rate and flow velocity combinations can be generated by setting volumetric flow rates to Q = 0.3, 1, 6, and 15 mL/min, respectively.
³Estimated from loading rates in Eamples 1 and 2.

Flow rates can be selected to produce mean (300 sec⁻¹) and maximal (2000 sec⁻¹) wall shear rates encountered in the femoral artery, as well as wall shear rates typical of veins (100 sec⁻¹) and stenotic vessels (5000 sec⁻¹) (Goldsmith and Turrito, (1986) Thromb Haemost 55:415-35). At the end of the recirculation time, the circuit can be washed with 20 mL of buffered saline, and the heparin-coated tubing segments eluted with SDS gel buffer as previously described. Samples can be run on Laemmli gels, and the quantity of surface bound ATIII for each condition determined by extrapolation from a standard curve included on each gel. Surface bound ATIII (ng) versus time of recirculation can be plotted for each ATIII and flow combination. On the basis of initial results from Examples 1 and 2, it is expected that loading of the endogenous alpha isoform to saturate early and to not increase as flow and wall shear rates increase. In contrast, endogenous beta isoform and DES.N135A surface loading are predicted to exhibit time and flow rate dependent increases, with recombinant DES.N135A ATIII increasing at a faster rate.

To react with heparin receptor molecules that are covalently attached to the tubing surface, ATIII molecules from the flowing bulk fluid phase must cross into the stationary diffusion layer located very close to the lumenal surface of the tubing. Then, once they are within this layer, the binding equilibrium constant for the molecular interaction of the ATIII with pentasaccharide sequences in the heparin can finally determine the extent of surface bound ATIII. Therefore, ATIII surface loading is a function of 1) flux from the flowing phase into the diffusion layer and 2) characteristics of the molecular level binding interaction between an ATIII molecule and its heparin receptor.
a) Modeling ATIII Loading onto the Heparin-Coated Surface of a Tubular Reactor Using a Two-Compartment Transport-Kinetic Model

ATIII loading onto the heparin-coated surface of a tubular reactor can be modeled using a two-compartment transport-kinetic model (Myszka, et al., (1998) Biophysical J 75:583-94). The simple assumption can be made that inside the flow channel, the ligand concentration far away from the reaction surface remains constant and is equal to inject value (C_{T}), whereas the ligand concentration near the surface (C_{S}) is uniform in space, but different from that in the “far-away” region. It can be assumed further that C_{S} rises rapidly over a short period of time after the flow starts, but changes very slowly thereafter (i.e., a quasi-steady-state approximation). The time evolution of ligand binding can be described by a simple ODE (eqn. 1) which has been routinely used for analyzing binding kinetics under the influence of mass transport in a BIAcore flow cell (Myszka, et al., (1998) Biophysical J 75:583-94).

$$ⅆ B \text{/} ⅆ t = \begin{matrix}( \frac{{k}_{a}}{1 + {k}_{a} ( {R}_{T} - B ) \text{/} {k}_{M}} {C}_{T} ( {R}_{T} - B ) ) - \\ ( \frac{{k}_{d}}{1 + {k}_{a} ( {R}_{T} - B ) \text{/} {k}_{M}} B )\end{matrix} \qquad ( eqn 1 )$$

In the above equation, B is bound ligand (i.e., ATIII molecules bound to heparin receptors on the tubing wall); C_{T} and R_{T} are the inject concentration of ligand (ATIII) and total receptor (heparin) concentration, respectively. Both are constants. kₐ and k_{d} are the association and dissociation rate constants for ligand-receptor binding (i.e., alpha or beta isoform or DES.N135A binding to heparin), which are known from equilibrium binding and rapid binding kinetic experiments (Turk, et al., (1997) Biochemistry 36:6682-91; and M. Jairajpuri and S. C. Bock, unpublished). k_{M} is the ligand's mass transfer coefficient, which is a function of the flow velocity of the bulk phase, Vz, and its diffusion constant, D. Since D is inversely related to molecular weight, it will be larger for 50,000 kDa recombinant DES.N135A than for the 60,000 kDa and 57,000 kDa plasma-derived alpha and beta-ATIIIs. Therefore, mass transport will be faster for lower molecular weight DES.N135A ATIII and beta-ATIII than for the major alpha-ATIII isoform. The mass transport coefficient k_{M} will be estimated as previously described (Cussler, E. (1984) Diffusion: Mass transfer in fluid systems. Cambridge University Press, New York, N.Y., p. 306). Hence, solutions to eqn. 1 can be obtained numerically as described (Myszka, et al., (1998) Biophysical J 75:583-94). Surface loading for the ATIIIs and flow conditions listed in Table 4 can be simulated. Comparison of simulation results and experimental data can allow evaluation and further refinement of the model for more accurately describing the contributions of mass transport and molecular binding interactions to ATIII surface loading.

### 2. Example 5b

### Loading of Endogenous Beta ATIII and DES.N135A ATIII Onto Heparin-Coated Surfaces Under Pulsatile, High Flow Conditions

The surface targeting properties of beta-ATIII and DES.N135A can be useful for several arterial applications, including reduction of thrombotic occlusion and proliferative restenosis following small-diameter vascular graft placement (e.g., femoro-popliteal bypasses), hemodialysis vascular access graft or fistula insertion, and angioplasty and/or stent placement. The ability of these ATIIIs to target and load thrombin/fXa inhibitory activity onto heparin/HSPG-coated surfaces can therefore be confirmed and characterized for systems with high pulsatile flow.

For this study a Harvard Apparatus Blood Pump and a Transonic flow meter with tubing flow sensor can be used. The pump simulates the ventricular action of the heart and can be used to conduct the studies described above under conditions of pulsatile, rather then steady flow. The systole/diastole ratio of the blood pump (mouse/rat model) is 35%/65%. The stroke volume can be adjusted from 50 μL to 1.0 mL, and the stroke rate from 20 to 200 per minute. Thus, overall flow rates of 1 to 200 mL/min can be achieved. The heparin-coated CBAS segment of the in vitro model flow system can be inserted into the Transonics tubing sensor to monitor flow rate (cm/sec). Based on the elevated beta to alpha isoform ratios observed in SDS extracts of uninjured and injured rabbit aorta endothelium (Witmer and Hatton, (1991) Arteriosclerosis and Thrombosis 11:530-9), it is expected that ATIIIs load onto the heparin-coated surface under pulsatile flow conditions, and that the higher affinity endogenous beta and recombinant DES.N135A ATIIIs will load more rapidly than the alpha isoform. Changes in the quantity (ng/cm²) of ATIII loading under pulsatile versus steady flow can be expected, but difficult to predict and model due to cyclic changes in the flow velocity, Vz, which is a component of the mass transport coefficient.

The principles shown to govern ATIII surface loading using the above described in vitro models can be further refined to model laminar flow at branch points, flow separations and constrictions, and expansions where occlusion and remodeling frequently occur (Malek, et D al., (1999) JAMA 282:2035-42).

### 3. Example 5c

### Loading of Endogenous ATIII Isoforms onto Native Vascular Surfaces in Different Regions of the Rabbit Vascular Tree

The anatomical distribution of the alpha and beta isoforms on surfaces from arterial versus venous regions of the rabbit circulatory tree can be investigated. The rabbit was the species in which ATIII isoforms were first discovered (Carlson and Atencio, (1982) Thromb Res 27:23-34). This experiment can be designed to obtain information about the radial distribution of the isoforms in different vessel wall layers because several lines of evidence suggest that a considerable portion of non-circulating ATIII is located subendothelially (deAgostini, et al., (1990) J Cell Biol 111:1293-1304; Carlson, et al., (1984) J Clin Invest 74:191-9; Carlson, et al., (1985) Blood 66:13-9; and Frebelius, Zuo, Lu, Swedenborg and Bock, unpublished observations).

Rabbits can be anesthetized with ketamine/xylazine (10 mg/kg ketamine and 3 mg/kg xylazine, i.v.) and perfused with normal saline. In this process they can be exsanguinated and euthanized. The aorta, vena cava, carotid arteries, jugular veins, and femoral arteries and veins can be removed and flushed with additional saline. Vessel segments can be opened longitudinally with scissors, and their lumenal surface area recorded. The endothelial layer can be lifted off with cellulose acetate paper. The intima/inner-media layer can be removed from the outer-media/adventitia layer by splitting through the natural cleavage plane between them. Endothelial cell, intima/inner-media and outer-medial/adventitia samples can then be extracted with 0.5 ml 0.5% SDS per cm² of lumenal surface area. Western blots can be prepared from 9% polyacrylamide Laemmli gels of reduced samples, and reacted with a sheep antibody to human ATIII that cross reacts with rabbit antithrombin.

The FIG. 2 in vitro flow model results indicates that rates of beta ATIII loading in arterial and pathological (high wall shear rate) flow regions are faster than in venous (low wall shear rate) regions, while the rate of alpha loading is slower and does not differ much with flow rate (or anatomical location). However, at “equilibrium” (120 minutes), the total amounts of surface-bound ATIII and the isoform ratios were similar for low and high flow sections of the circuit. Additional factors, beyond those modeled by the flow system can impact on how many ATIII molecules are associated with the wall at a particular site in the circulatory tree. For example, 1) the formation of T-AT and fXa-AT inhibitory complexes and 2) the cleavage of native ATIII by neutrophil elastase can both induce ATIII protein conformational changes that destroy the proteinase inhibitory activity, decrease heparin affinity, and lead to a reduction in the amounts of bound ATIII. Nevertheless, the availability of such information can be helpful for understanding the physiology of ATIII isoforms, and for evaluating potential clinical applications of super-beta-ATIII.

### F. Prophetic Example 6

### In Vivo Evaluation of a Recombinant Neutrophil Elastase-Resistant Beta ATIII for Thrombosis Reduction in Heparin-Coated Biomaterial and Native HSPG-Bearing Vascular Wall Applications

These studies can evaluate the potential of a neutrophil elastase-resistant recombinant beta ATIII (super-beta-ATIII) to decrease blood flow reductions in in vivo 1) biomaterial-based and 2) native thrombosis models. These studies are relevant to occlusion problems that are commonly encountered in small-diameter vascular graft, hemodialysis access graft, and indwelling catheter applications. These studies can use a rabbit arteriovenous shunt model to determine if priming a heparin-coated shunt with super-beta-ATIII, or intravenously administered super-beta ATIII, can reduce or prevent thrombotic occlusion. This can be relevant to reducing thrombotic occlusion in native arterial settings, such as after endarectomy, angioplasty stenting or bypass with natural conduits. These studies can use a rabbit carotid endothelial injury and stenosis model to evaluate the ability of super-beta-ATIII to reduce or eliminate cyclic flow reductions.

As noted above, super-beta-ATIIIs are recombinant ATIII molecules that inhibit thrombin and fXa with equal efficiency to plasma-derived ATIII in the absence and presence of heparin, but 1) bind heparin with 50-times higher affinity than the major alpha isoform of plasma ATIII, and 2) are 10-times more resistant to cleavage and inactivation by neutrophil elastase. Super-beta-ATIII can be used for Example 6 in vivo studies because it can offer an additional therapeutic advantage over the DES.N135A beta-ATIII that was used in the Examples described above. This advantage is related to removal of the neutrophil elastase cleavage site (Carrell and Owen, (1985) Nature 317:730-2) in the reactive center loop of super-beta-ATIII. Elastase cleavage of this site induces a protein conformational change that destroys ATIII proteinase inhibitor function. Focal and/or systemic inflammation, with neutrophil recruitment, activation and elastase production, is frequently encountered in patients with the problems that are being considered for treatment with a high heparin affinity recombinant beta ATIII like DES.N135A. Therefore, to prolong ATIII functional half-life in inflammatory milieus, super-beta-ATIII with the high heparin affinity of DES.N135A, but without its sensitivity to neutrophil elastase cleavage, will be used for these in vivo experiments.

Recombinant super-beta-ATIII and F122L.ATII (a control ATIII with 4000-fold reduced affinity for heparin (Jairajpuri, et al., (2003) J Biol Chem 278:15941-50) can be produced in the Drosophila expression system (DES, InVitrogen), which yields 5-15 mg purified ATIII per liter of induced cells. Pyrogenicity of ATIIIs used for animal experiments can be measured with the Limulus amebocyte assay, and further purification steps introduced if necessary.

### 1. Example 6a

### Investigation of ATIII Effects on Thrombosis in a Heparin-Coated Arteriovenous Shunt Model

Extracorporeal arteriovenous shunt models are widely utilized in thrombosis research for testing of biomaterial thrombogenicity and for evaluation of therapeutics (Harker, et al., (1991) Circulation 83 (Suppl. IV), 41-55). These shunt models are popular because they are technically simple and provide arterial flow of native blood without the confounding effects of anticoagulation. Thrombotic occlusion in shunt systems is due to the formation of platelet-rich, fibrin-containing, arterial-type thrombi within the shunt. A study using the thrombin specific inhibitor hirudin in a baboon shunt model established that the occlusive platelet-dependent thrombotic and hemostatic processes are mediated by thrombin (Kelly, et al., (1991) Blood 77:1006-12). The selective fXa inhibitors rTAP and DX-9065a also prevented thrombosis in a rat AV shunt model (Wong, et al., (1996) Thromb Res 83:117-26). Therefore, it can be appropriate to evaluate the targeted super-beta-ATIII thrombin and fXa inhibitor in an extracorporeal arteriovenous shunt model.
a) Rabbit Arteriovenous Shunt Model

The rabbit extracorporeal arteriovenous shunt model that can be used is a modification of that described by Valentin and coworkers (Valentin, et al., (1997) J Pharmacology and Experimental Therapeutics 280:761-9). In their original model, wall shear rates for 2 mm ID polyethylene carotid artery-jugular vein interposition shunts were about 600 sec⁻¹. Occlusion times were measured from temperature changes associated with initiation and cessation of blood flow in the extracorporeal shunt, and occurred at 13.7±1.3 min for controls.

In the proposed study, male New Zealand White rabbits (about 2.5 kg) can be used. Thirty-cm long shunts can be constructed from three segments of tubing. Two 12.5 cm×1.14 mm ID silicon-treated polyethylene catheters can be used for the end pieces. The central section can consist of a 6 cm×2 mm ID piece of uncoated PVC or heparin-coated PVC tubing (CBAS™ tubing). Blood flow rate through the shunt can be monitored continuously using a Transonics PXL tubing flow sensor attached to a Transonics TS410 tubing flowmeter. This strategy can allow the measurement of occlusion times as well as Fₓ values. Fₓ is the time for reduction of the flow rate to x % its average value during the first minute of flow through the shunt. Measurement of Fₓ rather than occlusion time can be preferable for experiments with heparin-coated shunts, which are expected to have longer baseline patency times than uncoated shunts. Heparin-coated shunts for the work on super-beta-ATIII surface targeting may be desired, but avoiding prolonged baseline occlusion times, which may introduce technical problems related to anesthesia duration and lengthy experiments, may be difficult. Therefore, pilot studies can be conducted 1) to measure the occlusion times of shunts made from 2 mm ID heparin-coated tubing, and 2) to determine the validity of using Fₓ rather than occlusion time (F₀) measurements. If the occlusion time is >30 minutes, 1) smaller bore heparin-coated tubing can be substituted for the central part of the shunt and/or 2) a piece of silk thread can be run through it to serve as a thrombogenic substrate.

For acute shunt experiments, rabbits can be anesthetized with ketamine/xylazine/butorphanol at 35 mg/kg IM, 5 mg/kg IM and 0.1 mg/kg IM, respectively. Duration of anesthesia is 90 minutes, and can be prolonged with an additional half dose of ketamine as needed. A shunt made of polyethylene and PVC tubing can be inserted between the left carotid artery and right jugular vein, and blood flow monitored until occlusion occurs. At the end of the experiment, rabbits can receive euthanasia. Euthanasia at the end of the experiment can be by barbituate overdose with intravenous (ear vein) Beuthanasia D at 1 cc per 10 pounds of body weight.
b) Shunt Pretreatment with ATIIIs

The central segments of the shunts can be made from uncoated or heparin-coated PVC tubing that has been pretreated for various periods of time with normal saline, t.ATIII, super-beta-ATIII, or F122L.ATIII. “t.ATIII” is human plasma-derived ATIII that contains the circulating ratio of ATIII isoforms, i.e., about 90% alpha to about 10% beta (t.ATIII can be isolated from outdated plasma by heparin-affinity and ion exchange chromatography steps. Typical yields are about 10 mg total ATIII from 100 cc outdated plasma). “Super-beta-ATIII” is recombinant neutrophil elastase-resistant beta ATIII as previously described. “F122L.ATIII” is a recombinant ATIII with a 4000-fold reduction in heparin binding affinity (Jairajpuri, et al., (2003) J Biol Chem 278:15941-50). A 10 cm length of tubing can be filled with saline or an ATIII, incubated for the indicated period of time, and washed with 20 mL normal saline. Then 2-cm pieces can be cut from each end of the treated tubing to use for measurement of surface bound ATIII by gel electrophoresis as shown in FIGS. 2 and 3. A tubing flow sensor can be placed around the remaining 6-cm pretreated tubing segment. This central portion of the shunt can be filled with saline and joined to the polyethylene end pieces of the shunt, whose outer termini have been inserted into the left carotid artery and right jugular vein. Clamps on the end pieces can be released to initiate flow through the shunt, and the flow rate continuously recorded by the flow sensor until occlusion (or a predetermined reduction in flow) occurs. Starting times for tubing pretreatment and the carotid artery/jugular vein dissection and catheterization procedures can be coordinated so that they end together, and the central and end segments of the shunt can be immediately joined to establish shunt flow.

To address whether pretreatment of heparin-coated shunts with ATIIIs prolong occlusion times, an experiment can be conducted with three groups of rabbits that receive heparin-coated shunts pretreated for 3 hours with saline or 1 μM t.ATIII or 1 μM super-beta-ATIII. The initial experiment can be conducted with 10 animals per group, and the means and standard deviations of occlusion times for the control and 2 experimental groups determined. Power analysis (95%) can be conducted using StatMate (GraphPad Software, Inc.) to estimate the number of animals required to detect a 50% increase in the occlusion time (or Fx) at p<0.05. Subsequent experiments can utilize these group sizes. In a previous rabbit study of thrombotic occlusion in silk thread-containing polyethylene shunts, a sample size of n=8 was sufficient to detect drug-dependent 50% increases in occlusion time at p<0.05 (Valentin, et al., (1997) J Pharmacology and Experimental Therapeutics 280:761-9). The occlusion time for saline pretreated shunts can be predicted to be faster than occlusion times of ATIII-pretreated shunts, and occlusion times for t.ATIII pretreated shunts can be predicted to be faster than those of super-beta-ATIII-pretreated shunts. Results of this sort would suggest that the inhibitory activities of surface-bound ATIII molecules, and ATIII molecules released into the blood-shunt surface interfacial zone, effectively neutralize thrombin, fXa and other activated coagulation factor enzymes that support occlusive platelet thrombosis and fibrin formation. Such results would suggest that early blockade of thrombotic amplification processes by surface bound enzyme inhibitors is a viable strategy for inhibiting occlusion of heparin-coated biomaterials.

To determine if super-beta-ATIII is more effective than plasma-derived ATIII for prolonging shunt occlusion times, and if the degree to which patency is extended is related to the degree of ATIII surface loading, the next experiment can be conducted with 9 groups. Shunts for these groups can be pretreated with saline, 1 μM t.ATIII or 1 μM super-beta-ATIII for 3 min, 30 min or 3 hours. Super-beta-ATIII can exhibit superior performance versus t.ATIII for loading shunts to high surface concentrations because, for any pretreatment time or concentration, its lower K_{d} for pentasaccharide can support a higher level of binding to the shunt surface.

For equivalent pretreatment times, it is expected that gel measurements of surface-bound ATIII will show super-beta-ATIII >>t.ATIII >>saline (0) (as in FIGS. 2 and 3), and that the occlusion times will also be in the order super-beta-ATIII >>t.ATIII >>saline. For a given ATIII, the surface-bound amounts should increase with longer pretreatment times, as should the occlusion times. Results of this sort indicate that more efficient loading of heparin-coated shunt surfaces with ATIII leads to more efficient inhibition of thrombotic occlusion pathways.

The shunt pretreatment experiment can addresses whether the occlusion time reduction requires a binding interaction between ATIII and heparin molecules attached to the shunt surface. This experiment is designed to interrupt the ATIII-heparin interaction from 2 different directions (both ligand and receptor), and will have 8 different groups. The 8 groups can receive shunts made of uncoated or heparin-coated PVC, which have been pretreated for equivalent times with saline or 1 μM t.ATIII or 1 μM super-beta-ATIII or 1 μM F122.ATIII. Again, surface bound ATIII and shunt occlusion times can be determined for each group. Predicted results for this experiment are tabulated below and would confirm that ATIII bound to heparin molecules on the surface of the tubing is responsible for increased shunt patency.

TABLE 5

| | Predicted | | |
|---|---|---|---|
| | surface | | Predicted |
| | bound ATIII/ | | surface |
| | occlusion | | bound ATIII/ |
| Tubing/ATIII | time | Tubing/ATIII | occlusion time |
| uncoated/saline | 0/<control | heparin-coated/saline | 0/control |
| uncoated/t.ATIII | 0/<control | heparin-coated/t.ATIII | +/>control |
| uncoated/super- | 0/<control | heparin-coated/super- | ++/>>control |
| beta | | beta | |
| uncoated/ | 0/<control | heparin-coated/ | 0/control |
| F122.ATIII | | F122.ATIII | |

This shunt experiment can be conducted to determine if simple pretreatment with high affinity ATIIIs represents a viable strategy for improving the patency of heparin-coated small-diameter vascular grafts, hemodialysis access grafts and indwelling catheters. This study can also provide dose response and mechanism information. Finally, the pretreatment approach can also be applicable to natural bypass graft conduits (e.g., saphenous vein), which bear pentasaccharide-containing HSPGs on their lumenal surfaces.
c) Effects of Intravenous ATIII Administration on Occlusion of Heparin-Coated Shunts

All shunts for Example 6 (3) can be made from heparin coated PVC and can not be pretreated with ATIIIs. Instead ATIIIs can be intravenously administered and required to bind to the shunt surface under arterial flow conditions. ATIIIs can be infused from sites just upstream of, or remote from, the shunt. The infusion site experiments can address the effects of hemodilution and competitive vascular wall HSPG binding of the adjunctive ATIII on shunt loading efficiency. Local infusion sites immediately upstream of the shunt can be prepared by inserting a polyethylene catheter into the cranial thyroid artery collateral of the left carotid artery. The catheter can be advanced until the ostium of the carotid artery is reached. Remote femoral vein infusion sites can be prepared by inserting a polyethylene catheter into the right femoral vein. To maintain patency of these catheters, saline can be continuously infused at a rate of 40 μL/min.

There can be 3 groups in the experiment which addresses 1) the ability of supplementary ATIII to prolong shunt occlusion time, and 2) the relative efficacy of super-beta-ATIII versus plasma-derived ATIIIs. These three groups can receive saline or 2 mg/mL t.ATIII or 2 mg/mL super-beta-ATIII administered proximally at 40 μL/min from the cranial thyroid artery site. Infusion can be switched from saline to the ATIII sample (or continued with saline in the case of the control) at 30 seconds prior to unclamping the shunt, and can continue for 12 minutes before switching back to saline. Thus, one milligram of ATIII can be delivered during this period. This one milligram dose is estimated to be about 5% of the ATIII amount which circulates freely in the blood of a 2.5 kg rabbit. As in previous experiments, flow can be continuously monitored with a tubing flow sensor mounted on the central heparin-coated PVC segment of the shunt, and used to determine occlusion time (or Fₓ). One half mL venous blood samples can also be collected from a marginal ear vein into one tenth volume of 3.8% sodium citrate prior to surgery and at 15 minutes post unclamping, and used for APTT and ATIII activity measurements.

Longer occlusion times for the ATIII infused groups versus the saline control group would indicate that a useful number of ATIII molecules were able to bind to the heparin-coated surface of the shunt under arterial flow conditions and delay the formation of occlusive thrombus. Longer occlusion times for the super-beta-ATIII group versus the t.ATIII group would support predictions from the faster loading of DES.N135A versus endogenous ATIII as observed in the FIG. 3 experiment, in which the recombinant beta-ATIII loaded onto the heparin-coated surface of the in vitro flow model about 9× more efficiently at high flow velocities than did endogenous ATIII. However, paradoxically a reciprocal relationship between ATIII activity levels in the circulating blood and shunt antithrombotic activity is expected. The combination of lower plasma ATIII activity levels and longer occlusion times for the super-beta-ATIII group would suggest that surface-bound and diffusion boundary layer thrombin and fXa molecules play a larger role in shunt thrombosis than do bulk fluid phase thrombin and fXa molecules. Alternatively, other patterns would force reconsideration of this hypothesis.

The degree of in vivo shunt loading should theoretically depend on several factors including: 1) the ATIII concentration of the infusate, 2) uptake of ATIII onto HSPG-bearing vascular surfaces of the circulatory tree, 3) consumption of ATIII via inhibitory complex formation with target enzymes and cleavage by neutrophil elastase, and 4) the ATIII concentration of blood flowing through the shunt. Therefore, proximity of the infusion site to the shunt can be considered. This experiment can have 4 groups receiving saline or 2 mg/mL super-beta-ATIII from remote femoral vein or proximal cranial thyroid artery infusion sites. It is expected that there will not be a significant differences in the occlusion times for the remote and proximal saline controls. Rabbits receiving super-beta-ATIII by femoral vein infusion can exhibit longer occlusion times than saline controls if the concentration of ATIII molecules making it into the shunt is not significantly depleted by hemodilution and binding to vascular surface HSPGs in the circulatory tree between the infusion site and the shunt. The occlusion times for the group with remote infusion are predicted to be shorter than for the group proximal infusion, and the degree of the difference can be an important factor in determining the site of administration in potential clinical applications. Large differences in occlusion times obtained with distal and proximal delivery would indicate that super-beta-ATIII needs to be infused near to its intended site of action. Modest differences in the occlusion times would indicate that pentasaccharide binding sites on vascular surface HSPG receptors in most of the circulatory system are fairly well saturated with ATIII and that super-beta-ATIII can be used as a systemic therapeutic.

To establish that antitthrombotic doses of super-beta-ATIII do not impair normal hemostasis (as occurs for example in the case of hirudin (Kelly, et al., (1991) Blood 77:1006-12), an additional experiment with 2 groups of rabbits can be conducted to determine if super-beta-ATIII promotes bleeding. One group can receive 1 mg of super-beta-ATIII (2 mg/mL×0.5 mL) by ear vein injection, and the other group can receive an equivalent volume of saline. Ear bleeding times (Herbert, et al., (1996) Circulation Res. 79:590-600) can be measured at 15 min post ATIII/saline dosing.

### 2. Example 6b

### Investigation of ATIII Effects on Thrombosis in a Carotid Stenosis and Endothelial Injury Model

These experiments can address the possibility of using super-beta-ATIII to reduce thrombotic occlusion in native arterial settings, such as after endarectomy, angioplasty, stenting or bypass with natural saphenous vein conduits. In these settings, the benefits of super-beta-ATIII would be derived from its ability to target and achieve better loading of endogenous HSPG-bearing vascular walls, and to provide early blockade of thrombin and fXa activities on lumenal surfaces and in the blood-vascular surface interfacial region where platelet activation, coagulation, signaling, and cell proliferative reactions are initiated.

A rabbit carotid endothelial injury and stenosis model can be used to evaluate the ability of super-beta-ATIII to reduce or eliminate thrombosis-induced cyclic flow reductions. The Folts cyclic flow reduction (CFR) model approximates the high shear rate conditions of a pathologically stenosed artery with endothelial damage. Thrombus formation is platelet-dependent and mediated by thrombin, as demonstrated by the ability of the thrombin specific inhibitor hirudin to reduce CFR frequency and increase blood flow (Rubsamen, et al., (1995) Thromb Haemost 74:1353-60). Therefore, like the arteriovenous shunt model, the Folts CFR model represents a valid experimental strategy for evaluating a surface-targeted thrombin/fXa inhibitor such as super-beta-ATIII.

A rabbit Folts model can be used for Example 6b. In this model, mechanical injury of the endothelium combined with critical stenosis induces cyclic flow reductions with pre-stenosis shear rates of about 600 sec⁻¹ and post-stenosis shear rates in the 20,000 to 60,000 sec⁻¹ range (Valentin, et al., (1997) J Pharmacology and Experimental Therapeutics 280:761-9; Lott, et al., (1998) J of Thrombosis and Thrombolysis 5:15-23). Rabbits can be prepared as described for the shunt model of Example 5a, with an infusion catheter inserted into the cranial thyroid artery and delivering normal saline at 40 μL/min. For monitoring blood flow velocity, a Transonics 2SB 2 mm perivascular flow probe (attached to a Transonics TS420 perivascular flowmeter) can be placed on the carotid artery proximal to the cranial thyroid artery. Endothelial injury can be induced in a carotid artery segment distal to the flow probe and the cranial thyroid artery by repeated clamping with hemostatic forceps. Then an inflatable vascular occluder (FST) can be placed around the injured segment. While blood flow is monitored, the occluder can be inflated until the vessel lumen cross sectional area is constricted enough that cyclic flow reductions are observed. Then blood flow can be recorded for 5 CFRs (about 30 min) to establish the basal CFR frequency. Technique and model function can be validated in several control rabbits. Epinephrine (0.5 μg/min) can be administered as a positive control to sensitize platelets to other agonists and accelerate CFR formation. Then the antiplatelet agents aspirin (12.5 mg) and ketanserin (0.625 mg) can be administered to abolish CFRs and serve as a negative control.

Initially, three experimental groups (n=8) can be used for Example 6b. Sample size calculations (Statmate, GraphPad Software, Inc.) using means and standard deviations of CFRs from a published rabbit Folts model study (Lott, et al., (1998) J of Thrombosis and Thrombolysis 5:15-23) indicate that a sample size of n=8 can allow detection of changes of >20% in the CFR frequency with 95% confidence at p<0.05. After recording 5 baseline CFRs, rabbits in each of these groups can receive 12.5 minute infusions of saline or 2 mg/mL t.ATIII or 2 mg/mL super-beta-ATIII at 40 μL/min through the intracranial thyroid artery catheter, followed by saline infusion when the sample infusion ends. Monitoring of blood flow patterns can continue during the period of infusion and for about 1 hour thereafter. Average CFR frequency and slope can be calculated for baseline CFRs obtained during the pre-infusion period, and compared with CFRs and flow patterns obtained during and after sample infusion. For the control group, the frequency and slopes of CFRs during and after saline infusion should be similar to the baseline pattern. If the administered doses of ATIII do not diminish stenosis-generating thrombin and fXa activities significantly, CFR patterns during and after ATIII infusion can also be similar to the baseline period, as observed for the control saline sample.

If ATIII administration reduces thrombin and fXa enzymatic activities in the region of the stenosis to levels supporting platelet aggregate and fibrin formation less well, CFRs during and for an interval after sample infusion will be longer and have shallower slopes. The time required for return to the baseline CFR frequency and slope will reflect how long effective concentrations of ATIII are maintained at the stenosis. If the improved surface loading and neutrophil elastase properties of super-beta-ATIII allow it to target thrombin/fXa enzymatic activities in the stenosis better, one will observe, compared to an equivalent dose of t.ATIII, a CFR pattern with shallower slopes and increased durations that can or cannot be sustained for a longer period of time after sample infusion. While not wishing to be bound by theory, the enhanced surface loading property of super-beta-ATIII is expected to promote more efficient thrombin and fXa inhibition in the region of the stenosis, which should immediately reduce the slopes and increase the durations of CFRs. However, paradoxically, the interval until return to the baseline CFR pattern can be shortened since rapid uptake of super-beta-ATIII by vascular surfaces in the rest of the body can rapidly deplete it from the blood. Therefore, less super-beta-ATIII can be available for binding during subsequent passes through the stenosis. These considerations suggest that proximal intravenous infusion of super-beta-ATIII can be a suitable way to administer it therapeutically.

Example 6b can use the Folts model of endothelial damage and stenosis induced high shear rate thrombus formation to determine whether the improved surface loading and neutrophil elastase-resistance properties of super-beta-ATIII are advantageous in an application where the receptors are native HSPG-bearing surfaces, rather than heparin-coated biomaterials. The Folts model is relevant to clinical thrombosis based on the observation of CFRs following coronary angioplasty in humans (Anderson, et al., (1994) J Am Coll Cardiol 23:1031-7), and it has been widely used for testing antithrombotic drugs in vivo. In this model, thrombosis is induced by two factors, mechanical damage of the endothelium and vessel constriction. The endothelial damage step exposes the subendotheial layer of the vessel wall, which has been demonstrated to contain a higher density of ATIII binding sites than the uninjured lumenal endothelial surface by clearance (Carlson, et al., (1984) J Clin Invest 74:191-9; Carlson, et al., (1985) Blood 66:13-9) and ¹²⁵I-ATIII perfusion (Agostini, et al., (1990) J Cell Biol 111:1293-1304) studies. Thus, as a consequence of endothelial damage, there may be an opportunity for increased vascular surface loading of ATIIIs, and especially super-beta-ATIII. Endothelial damage and subendothelium exposure are inherent in endarectomy, angioplasty and stenting procedures and in bypass graft saphenous vein harvest and preparation. Although these interventions effectively restore cardiovascular blood flow, they are also associated with significant reocclusion, rethrombosis and restenosis rates. Therefore a vessel wall targeted therapeutic that could inhibit acute thrombotic occlusion (and longer term restenosis) would be of clinical significance for many cardiovascular patients.

Rabbits can be anesthetized as above for Example 6a. An infusion catheter can be inserted into the cranial thyroid artery to deliver normal saline at 40 μL/min. For monitoring blood flow velocity, a perivascular flow probe can be placed on the carotid artery proximal to the cranial thyroid artery. Endothelial injury can be induced in a carotid artery segment distal to the flow probe and the cranial thyroid artery by repeated clamping with hemostatic forceps. Then an inflatable vascular occluder can be placed around the injured segment. While blood flow is monitored, the occluder can be inflated until the vessel lumen cross sectional area is constricted enough that cyclic flow reductions are observed. Blood flow can be recorded for 5 CFRs (about 30 min) to establish the basal CFR frequency. Animals can next receive 12.5 minute infusions of saline or ATIII at 40 μL/min through the intracranial thyroid artery catheter, followed by saline infusion when the sample infusion ends. Monitoring of blood flow patterns can continue during the period of infusion and for about 1 hour thereafter. Euthanasia at the end of acute experiments can be by barbituate overdose with intravenous (ear vein) Beuthanasia D at 1 cc per 10 pounds of body weight. This method is consistent with the recommendations of the Panel on Euthanasia of the American Veterinary Medical Association.
a) Comment on Super-Beta-ATIII Restenosis Applications

Thrombin is an important serine proteinase with diverse substrates situated at key regulatory points in a large number of physiologically critical pathways. With regard to cardiovascular occlusion and stenosis, thrombin contributes to platelet activation, fibrin polymerization, PAR signaling, and smooth muscle cell proliferation. The surface targeted anti-thrombin and anti-fXa activities of super-beta-ATIII can allow early intervention in all of the above pathways and attenuate associated pathologies by reducing thrombin enzymatic activity and thrombin generation in the critical blood-vascular surface interfacial region. The disclosure herein, in one aspect, focused on evaluation of super-beta-ATIII for the treatment of acute occlusive thrombosis.

### G. Example 7

### Disruption of Structural Interactions Between Helix D and Strand 2A of Native ATIII to Produce Further Enhancement of ATIII Heparin Affinity and Basal fXa Inhibition

Heparin cofactor binding to native ATIII extends helix D C-terminally by one turn. Extension of the alpha helix in AT*H repositions tyrosine-131, and breaks up native van der Waals and hydrophobic interactions of its sidechain ring with asn-127 and leucine-130 in helix D and leucine-140 and serine-142 in strand 2A (FIG. 12). Heparin-mediated helix D extension and associated protein conformational changes occurring through-out the ATIII molecule lead to activation of fXa inhibitory activity and increased affinity for heparin.

Genetic disruption of native conformation Y131 ring interactions with hD and strand 2A similarly increase ATIII fXa inhibition and affinity for heparin, but do so independently of cofactor binding. Relative to their DES.N135A super-beta ATIII parent molecule, Y131A and Y131L substitution mutants exhibit 30-fold increased rates of progressive (heparin-independent) fXa inhibition and about 5-fold increased affinity for heparin. Y131A and Y131L also exhibit an about 8% increase in basal fluorescence and correspondingly reduced heparin-dependent fluorescence enhancement. These properties suggest that disruption of the native conformation Y131-N127-L130-L140-S145 cluster shifts the position of the native to activated conformational equilibrium towards the activated conformation. The heparin-independent factor Xa inhibition rate, heparin binding affinity, basal fluorescence and heparin-dependent fluorescence enhancement of the Y131F mutant and the DES.N135A super-beta ATIII parent molecule were similar. Together, these data indicate that the Y131 ring, but not its hydroxyl group, is critical for stabilizing the native, reactive center loop-inserted conformation of ATIII that is a poor factor Xa inhibitor.

Tyrosine-131 substitution with leucine or alanine on a DES.N135A super-beta ATIII background increased heparin binding affinity by 5-fold relative to the recombinant super-beta ATIII parent, by 50-fold relative to the minor plasma-derived beta ATIII: isoform, and by 250-fold relative to the major plasma-derived alpha ATIII isoform. The extremely enhanced affinity of these ATIII variants can facilitate even better targeting of antithrombins anticoagulant, antiproliferative and antiinflammatory activities to heparin- and HSPG-coated surfaces under static and low and high wall shear rate flow conditions. The increased fXa inhibition activity of these variants can also provide improved regulation of systemic activated fXa.

### H. Example 8

### Preloading Medical Devices Prior to Implantation with Super Beta ATIII

This example determines if ATIII pretreatment of extracorporeal rabbit carotid artery-jugular vein shunts prolongs patency and investigates the ATIII dose dependence and heparin binding affinity dependence of the time to thrombotic occlusion.

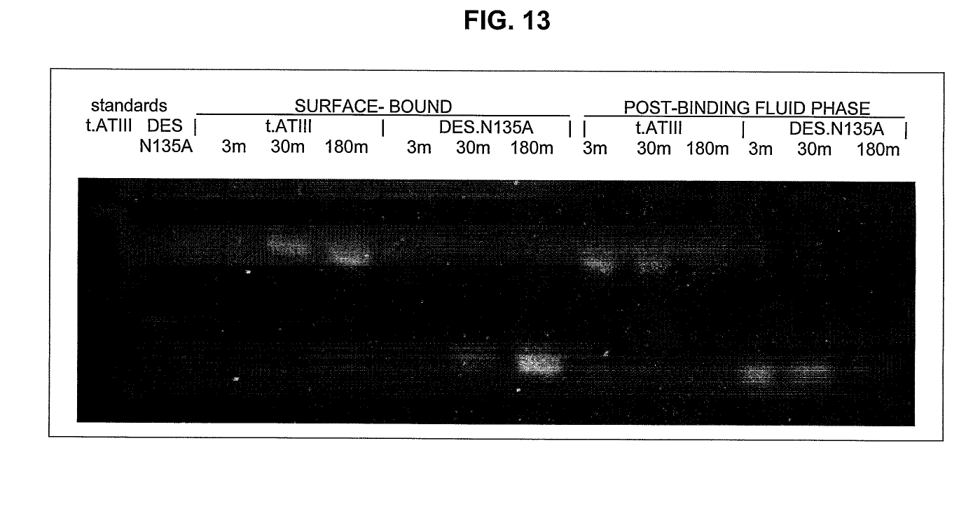

A study was conducted to test the proposed strategy of preloading ATIII onto shunt surfaces under static, no-flow conditions. Segments of heparin-coated 3 mm ID CBAS™ tubing were filled with 1 μM solutions of plasma-derived t.ATIII or recombinant DES.N135A ATIII. After 3 min, 30 min, or 3 hours, the ATIII solutions were recovered. The tubing pieces were washed with normal saline, then lumenal surface-bound protein was eluted with SDS gel buffer. Surface-bound and post-binding fluid phase samples were analyzed by Laemmli gel electrophoresis as shown in FIG. 13.

The amount of surface-bound ATIII increased as a function of the pretreatment time and was about 3-fold higher for tubing segments that had been treated with the enhanced affinity recombinant ATIII rather than plasma-derived ATIII. Reciprocal depletion of ATIII from the post-binding fluid phase was also observed. These data illustrate a simple and efficient strategy for preloading the surfaces of heparin-coated vascular grafts (or saphenous vein bypass grafts) with a thrombin and fXa inhibitor.

### I. Specific Embodiments

In one aspect, disclosed herein are methods of decreasing coagulation or thrombosis in a system, comprising administering an ATIII molecule to the system, wherein the ATIII molecule has an increased affinity for heparin or heparan sulfate proteoglycans bound to a solid surface, and wherein the ATIII binds the heparin or heparan sulfate proteoglycans under high wall shear rate conditions with a higher affinity than alpha ATIII. In one example, the no methods disclosed herein can farther comprise a determination that high wall shear rates on the ATIII will occur.

In another aspect, disclosed herein are methods of inhibiting coagulation under low and high wall shear rate conditions comprising administering an ATIII molecule, wherein the ATIII molecule binds heparin or heparan sulfate proteoglycans under low and high wall shear rate conditions with an affinity higher than alpha ATIII.

In yet another aspect, disclosed herein are methods of inhibiting coagulation or thrombosis during or following a cardiovascular procedure on a subject comprising administering high affinity ATIII molecules to the subject, wherein the ATIII molecules bind heparin or heparan sulfate proteoglycans under low and high wall shear rate conditions with an affinity higher than alpha ATIII. In one example, the ATIII can be administered upstream of the area where ATIII loading is desired. In another example, the method can further comprise administering heparin or heparan sulfate proteoglycans.

In a further aspect, disclosed herein are methods of preconditioning a heparin or heparan sulfate polyglycan coated material comprising incubating the material with a solution comprising ATIII molecules, such that the ATIII molecules bind to the heparin or heparan sulfate proteoglycans under low and high wall shear rate conditions with an affinity higher than alpha ATIII.

In still a further aspect, disclosed herein are methods of determining an amount of heparin or HSPG on a surface, comprising contacting the surface with a composition comprising an ATIII molecule at a shear rate, wherein the ATIII molecule has an increased affinity for heparin or heparan sulfate proteoglycans; and assaying the amount of the ATIII molecule bound to the surface, the amount of the ATIII bound to the surface being the minimum amount of heparin or heparan sulfate proteoglycan on the surface. In a specific example, the surface can be contacted with an excess of the ATIII molecule, the amount of ATIII bound to the surface being the amount of heparin or heparan sulfate proteoglycan bound to the surface.

In yet a further aspect, disclosed herein are methods of determining a wall shear rate on a heparin or HSPG coated surface comprising contacting the surface with a composition comprising an ATIII molecule, wherein the ATIII molecule has an increased affinity for heparin or heparan sulfate proteoglycan; and assaying the amount of ATIII bound to the surface, the higher the amount of ATIII bound to the surface the higher the wall shear rate.

In another aspect, disclosed herein are methods of coating a surface with heparin or heparan sulfate proteoglycan comprising determining an amount of ATIII that binds to the surface, where the ATIII has a high affinity for heparin or heparan sulfate proteoglycan; and coating the surface with heparin or heparan sulfate proteoglycan in an amount at least that of ATIII bound to the surface.

In the methods disclosed herein, the systems can comprise heparin or heparan sulfate proteoglycans attached to a solid surface. The solid surface can comprise a biomaterial. In other specific examples, the system can comprise a stent, a heart pump, a heart lung bypass machine, a blood oxygenator, a ventricular assist device, a ventricular graft, a catheter, an extracorporeal circuit, a blood gas sensor, an intraocular lens, or a heparin coated thermoplastic.

In another example of the methods disclosed herein, the ATIII can comprise a beta-ATIII. The dissociation constant of ATIII for heparin can be less than or equal to 54 nM, or 6 nM, or 1 nM. The ATIII can be produced in an insect or yeast expression system.

In yet another example of the methods disclosed herein, the shear rate conditions can comprise shear rates of at or at least 50, 630, 1000, 1500, 2000, 2500, 3000, or 3500 sec⁻¹.

Also disclosed herein is a strategy for increasing ATIII heparin binding affinity and rate of basal factor Xa inhibition by disrupting interactions between helix D and strand 2A of the native molecule.

Also disclosed herein are compositions of antithrombins for which the heparin binding affinity and basal factor Xa rate are increased by disrupting interactions between helix D and sheet A of the native molecule.

### J. REFERENCES

Agostini, A. d., Watkins, S., Slayter, H., Youssoufian, H., and Rosenberg, R. (1990) Localization of anticoagulantly active heparan sulfate proteoglycans in vascular endothelium: Antithrombin binding on cultured endothelial cells and perfused rat aorta J Cell Biol. 111:1293-1304.
Ali, M., Mazur, W., Kleinman, N., Rodgers, G., Abukhalil, J., French, B., and Rainzer, A. (1996) Inhibition of coronary restenosis by antithrombin III in atherosclerotic swine Coronary Artery Dis. 7:851-61.
Anderson, H., Kirkeeide, R., Krishnaswami, A., Weigelt, L., Revana, M., Weisman, H., and Willerson, J. (1994) Cyclic flow variations after coronary angioplasty in humans: clinical and angiographic characteristics and elimination with 7E3 monoclonal antiplatelet antibody J. Am. Coll. Cardiol. 23:1031-7.
Backovic, M., and Gettins, P. (2002) Insight into residues critical for antithrombin function from an expanded database of sequences that includes frog, turtle and ostrich antithrombins J Proteome Res. 1:367-73.
Bayston, T., Tripodi, A., Mannucci, P., Thompson, E., Ireland, H., Fitches, A., Hananeia, L., Olds, R., and Lane, D. (1999) Familial overexpression of beta antithrombin caused by an Asn135Thr substitution Blood 93:4242-7.
Begovac, P., Thomson, R., Fisher, J., Hughson, A., and Gallhagen, A. (2003) Improvements in GORE-TEX vascular graft performance by Carmeda bioactive surface heparin immobilization Eur. J. Vasc. and Endovasc. Surg. 25:432-7.
Blezer R, Fouache B, Willems G M, Lindhout T. (1997) Activation of blood coagulation at heparin-coated surfaces. J Biomed Mater Res. 37:108-13.
Brennan, S., George, P., and Jordan, R. (1987) Physiological variant of antithrombin-III lacks carbohydrate sidechain at Asn 135 FEBS Lett. 219:431-6.
Bjork, I., Ylinenjarvi, K., Olson, S. T., Hermentin, P., Conradt, H. S., Zettlmeissl, G. (1992) Decreased affinity of recombinant antithrombin for heparin due to increased glycosylation Biochem J 286 (Pt 3):793-800.
Carlson, T., Atencio, A., and Simon, T. (1985) Comparison of the behavior in vivo of two molecular forms of antithrombin III Biochem. J. 225:557-64.
Carlson, T., Simon, T., and Atencio, A. (1985) In vivo behavior of human radioiodinated antithrombin III: distribution among three physiologic pools Blood 66:13-9.
Carlson, T., Atencio, A., and Simon, T. (1984) In vivo behavior of radioiodinated rabbit antithrombin III. Demonstration of a noncirculating vascular compartment J Clin Invest. 74:191-9
Carlson, T., and Atencio, A. (1982) Isolation and partial characterization of two distinct types of antithrombin III from rabbit Thromb Res. 27:23-34.
Carrell, R., and Owen, M. (1985) Plakalbumin, alpha1-antitrypsin, antithrombin and the mechanism of inflammatory thrombosis Nature 317:730-2.
Cohen, J., Tenenbaum, N., Sarfati, I., Tyras, D., Graver, L., Weinstein, G., and Wise, L. (1992) In vivo inactivation of antithrombin III is promoted by heparin during cardiopulmonary bypass J. Invest. Surg. 5:45-9.
Cussler, E. (1984) Diffusion: Mass transfer in fluid systems. Cambridge University Press, New York, N.Y.
deAgostini, A., Watkins, S., Slayter, H., Youssoufian, H., and Rosenberg, R. (1990) Localization of anticoagulantly active heparan sulfate proteoglycans in vascular endothelium: Antithrombin binding on cultured endothelial cells and perfused rat aorta J. Cell Biol. 111:1293-1304.
Devine, C., Hons, B., and McCollum, C. (2001) Heparin-bonded dacron or polytetrafluoroethylene for femoropipliteal bypass grafting: a multicenter trial J. Vasc. Surg. 33:533-9.
Dewerchin, M., Hérault, J.-P., Wallays, G., Petitou, M., Schaeffer, P., Millet, L., Weitz, J., Moons, L., Collen, D., Carmeliet, P., and Herbert, J.-M. (2003) Life-threatening thrombosis in mice with targeted Arg48-to-Cys mutation of the heparin-binding domain of antithrombin Circ. Res. 93.
Dunzendorfer, S., Kaneider, N., Rabensteiner, A., Meierhofer, C., Reinisch, C., Römisch, J., and Wiedermann, C. (2001) Cell-surface heparan sulfate proteoglycanmediatedregulation of human neutrophil migration by the serpin antithrombin III Blood 97:1079-85.
Ersdal-Badju, E., Lu, A., Peng, X., Picard, V., Zendehrouh, P., Turk, B., Björk, I., Olson, S., and Bock, S. (1995) Elimination of glycosylation heterogeneity affecting heparin affinity of recombinant human antithrombin III by expression of a beta-like variant in baculovirus-infected insect cells. Biochem. J. 310:323-30.
Frebelius, S., Isaksson, S., and Swedenborg, J. (1996) Thrombin inhibition by antithrombin III on the subendothelium is explained by the isoform AT-beta Thromb. Vasc. Biol. 16:1292-7.
Garone, L., Edmunds, T., Hanson, E., Bernasconi, R., Huntington, J. A., Meagher, J. L., Fan, B., Gettins, P. G. (1996) Antithrombin-heparin affinity reduced by fucosylation of carbohydrate at asparagine 155 Biochemistry 35(27):8881-9.
Goldsmith, H., and Turrito, V. (1986) Rheological aspects of thrombosis and haemostasis: Basic principles and applications Thromb. Haemost. 55:415-35.
Goldstein, D. (2003) Worldwide experience with the MicroMed DeBakey ventricular assist device as a bridge to transplantation Circulation 108 (suppl. II), 272-7.
Grip, L., Blomback, M., Egberg, N., Olsson, A., Svane, B., and Velander, M. (1997) Antithrombin III supplementation for patients undergoing PTCA for unstable angina pectoris. A controlled randomized double-blind pilot study Eur. Heart J 18:443-9.
HajMohammadi, S., Enjyoji, K., Princivalle, M., Christi, P., Lech, M., Beeler, D., Rayburn, H., Schwartz, J., Barzegar, S., Agostini, A. d., Post, M., Rosenberg, R., and Shworak, N. (2003) Normal levels of anticoagulant heparan sulfate are not essential for normal hemostasis J. Clin. Invest. 111:989-99.
Harker, L., Kelly, A., and Hanson, S. (1991) Experimental arterial thrombosis in nonhuman primates Circulation 83 (Suppl. IV), 41-55.
Herbert, J., Herault, J., Bernat, A., vanAmsterdam, R., Vogel, G., Lormeau, J., Petitou, M., and Meuleman, D. (1996) Biochemical and pharmacological properties of SANORG 32701. Comparison with the ‘synthetic pentasaccharide’ (SR 90107/ORG 31540) and standard heparin Circulation Res. 79:590-600.
Hoffmann, J., Vollmar, B., Römisch, J., Inthorn, D., Schildberg, F., and Menger, M. (2002) Antithrombin effects on endotoxin-induced microcirculatory disorders are mediated mainly by its interaction with microvascular endothelium Crit. Care Med. 30, 218-25.
Jairajpuri, M., Lu, A., Desai, U., Olson, S., Bjork, I., and Bock, S. (2003) Antithrombin III phenylalanines 122 and 121 contribute to its high affinity for heparin and its conformational activation J. Biol. Chem. 278:15941-50.
Kelly, A., Marzec, U., Krupski, W., Bass, A., Cadroy, Y., Hanson, S., and Harker, L. (1991) Hirudin interruption of heparin-resistant arterial thrombosis formation in baboons Blood 77:1006-12.
Kocsis, J., Llanos, G., and Holmer, E. (2000) Heparin-coated stents J. of Long-Term Effects of Medical Implants 10: 19-45.
Larm, O., Larsson, R., and Olsson, P. (1983) A new nonthrombogenic surface prepared by selective covalent binding of heparin via a modified reducing terminal residue Biomater. Med. Dev. Artif. Organs 11:161-3.
Lott, F., Nelson, A., and Toombs, C. (1998) Effects of recombinant human megakaryocyte growth and development factor (rHuMGDF) on platelet production, platelet aggregation and thrombosis J. of Thrombosis and Thrombolysis 5:15-23.
Malek, A., Alper, S., and Izumo, S. (1999) Hemodynamic shear stress and its role in athersclerosis JAMA 282:2035-42.
Myszka, D., He, X., Dembo, M., Morton, T., and Goldstein, B. (1998) Extending the range of rate constants available for BIACORE: Interpreing mass transport influenced binding data Biophysical J 75:583-94.
Oelschläger, C., Römisch, J., Staubitz, A., Stauss, H., Leithäuser, B., Tillmanns, H., and Hölschermann, H. (2002) Antithrombin III inhibits nuclear factor kappa B activation in human monocytes and vascular endothelial cells Blood 99, 4015-20.
Olson, S. T., Frances-Chmura, A. M., Swanson, R., Bjork, I., Zettlmeissl, G. (1997) Effect of individual carbohydrate chains of recombinant antithrombin on heparin affinity and on the generation of glycoforms differing in heparin affinity Arch Biochem Biophys 341(2):212-21.
Peterson, C., and Blackburn, M. (1985) Isolation and characterization of an antithrombin III variant with reduced carbohydrate content and enhanced heparin binding J. Biol. Chem. 260:610-5.
Picard, V., Ersdal-Badju, E., and Bock, S. (1995) Partial glycosylation of antithrombin III asparagine-135 is caused by the serine in the third position of its N-glycosylation consensus sequence and is responsible for production of beta-antithrombin III isoform with enhanced heparin affinity Biochemistry 34:8433-40.
Rothenburger, M., Wilhelm, M., Hammel, D., Schmidt, C., Tjan, T., Bocker, D., Scheld, H., and Schmid, C. (2002) Treatment of thrombus formation associated with theMicroMed DeBakey VAD using recombinant tissue plasminogen activator Circulation 106 [suppl I], I-189-192.
Rubsamen, K., Hornberger, W., and Kirchengast, M. (1995) Inhibition of arterial thrombus formation in two canine models: comparison of ancrod, a fibrinogen-depleting agent, the thrombin-inhibitor r-hirudin, and the glycoprotein 11b/IIIa-receptor antagonist Ro 43-8857 Thromb Haemost. 74:1353-60.
Turk, B., Brieditis, I., Bock, S., Olson, S., and Bjork, I. (1997) The oligosaccharide side chain on Asn-135 of alpha-antithrombin, absent in beta-antithrombin, decreases the affinity of the inhibitor by affecting the heparin-induced conformational change Biochemistry 36:6682-91.
Valentin, J., Vieu, S., Bertolino, F., Faure, P., and John, G. (1997) Differential involvement of serontonin 2A/C and thromboxane A2/Prostanoid receptors in high- vs. low-shear rate arterial thrombosis in rabbits J. Pharmacology and Experimental Therapeutics 280:761-9.
Weitz, J. (2003) Heparan sulfate: Antithrombotic or not? J. Clin. Invest. 111:952-4.
Witmer, M., and Hatton, M. (1991) Antithrombin III-beta associates more readily than antithrombin III-alpha with uninjured and de-endothelialized aortic wall in vitro and in vivo Arteriosclerosis and Thrombosis 11:530-9.
Wong, P., Crain, E., O, O. N., Watson, C., and Racanelli, A. (1996) Antithrombotic actions of selective inhibitors of blood coagulation factor Xa in rat models of thrombosis Thromb Res. 83:117-26.
Lawrence, D., Ginsburg, D., Day, D., Berkenpas, M., Verhamme, I., Kvassman, J., and Shore, J. (1995) Serpin-protease complexes are trapped as stable acyl-enzyme intermediates J Biol Chem 270:25309-12.
Wilczynska, M., Fa, M., Ohlsson, P.-I., and Ny, T. (1995) The inhibition mechanism of serpins. Evidence that the mobile reactive center loop is cleaved in the native protease-inhibitor complex J Biol Chem 270:29652-5.
Huntington, J., Read, R., and Carrell, R. (2000) Structure of a serpin-protease complex shows inhibition by deformation Nature 407:923-6.
Olson, S., Swanson, R., Raub-Segall, E., Bedsted, T., Sadri, M., Petitou, M., Herault, J., Herbert, J., and Bjork, I. (2004) Accelerating ability of synthetic oligosaccharides on antithrombin inhibition of proteinases of the clotting and fibrinolytic systems. Comparison with heparin and low-molecular-weight heparin Thrombosis and Haemostasis 92(5):929-39.
Schreuder, H., deBoer, B., Dijkema, R., Mulders, J., Theunissen, H., Grootenhuis, P., and Hol, W. (1994) The intact and cleaved human antithrombin III complex as a model for serpin-proteinase interactions Struct Biol 1:48-54.
Jin, L., Abrahams, J., Skinner, R., Petitou, M., Pike, R., and Carrell, R. (1997) The anticoagulant activation of antithrombin by heparin Proc Natl Acad Sci USA 94:14683-8.
Skinner, R., Abrahams, J., Whisstock, J., Lesk, A., Carrell, R., and Wardell, M. (1997) The 2.6A structure of antithrombin indicates a conformational change at the heparin binding site J Mol Biol 266:601-9.
Desai, U., Swanson, R., Bock, S., Bjork, I., and Olson, S. (2000) Role of arginine 129 in heparin binding and activation of antithrombin J Biol Chem 275:18967-84.
Schedin-Weiss, S., Desai, U., Bock, S., Gettins, P., Olson, S., and Bjork, I. (2002) Importance of lysine 125 for heparin binding and activation of antithrombin Biochemistry 41:4779-88.
Jairajpuri, M., Lu, A., Desai, U., Olson, S., Bjork, I., and Bock, S. (2003) Antithrombin III phenylalanines 122 and 121 contribute to its high affinity for heparin and its conformational activation J Biol Chem 278:15941-50.
Arocas, V., Turk, B., Bock, S., Olson, S., and Bjork, I. (2000) The region of antithrombin interacting with full-length heparin chains outside the high-affinity pentasaccharide sequence extends to Lys136 but not to Lys139 Biochemistry 39:8512-8.
Meagher, J., Beechem, J., Olson, S., and Gettins, P. (1996) Role of arginine 132 and lysine 133 in heparin binding to and activation of antithrombin J Biol Chem 271:29353-8.
Belzar, K., Zhou, A., Carrell, R., Gettins, P., and Huntington, J. (2002) Helix D elongation and allosteric activation of antithrombin J Biol Chem 277:8551-8.

### K. Sequences

1. SEQ ID NO:1 Protein sequence Human ATIII
Accession no. AH004913.
MYSNVIGTVTSGKRKVYLLSLLLIGFWDCVTCHGSPVDICTAKPRDIPMN
PMCIYRSPEKKATEDEGSEQKIPEATNRRVWELSKANSRFATTFYQHLAD
SKNDNDNIFLSPLSISTAFAMTKLGACNDTLQQLMEVFKFDTISEKTSDQ
IHFFFAKLNCRLYRKANKSSKLVSANRLFGDKSLTFNETYQDISELVYGA
KLQPLDFKENAEQSRAAINKWVSNKTEGRITDVIPSEAINELTVLVLVNT
IYFKGLWKSKFSPENTRKELFYKADGESCSASMMYQEGKFRYRRVAEGTQ
VLELPFKGDDITMVLILPKPEKSLAKVEKELTPEVLQEWLDELEEMMLVV
HMPRFRIEDGFSLKEQLQDMGLVDLFSPEKSKLPGIVAEGRDDLYVSDAF
HKAFLEVNEEGSEAAASTAVVIAGRSLNPNRVTFKANRPFLVFIREVPLN
TIIFMGRVANPCVK

```
2. SEQ ID NO:2 cDNA sequence Human ATIII Accession no. I03102.
      1 caccagcatc atctcctcca attcatccag ctactctgcc catgaagata atagttttca
    61 ggcggattgc ctcagatcac actatctcca cttgcccagc cctgtggaag attagcggcc
  121 atgtattcca atgtgatagg aactgtaacc tctggaaaaa ggaaggttta tcttttgtcc
  181 ttgctgctca ttggcttctg ggactgcgtg acctgtcacg ggagccctgt ggacatctgc
  241 acagccaagc cgcgggacat tcccatgaat cccatgtgca tttaccgctc cccggagaag
  301 aaggcaactg aggatgaggg ctcagaacag aagatcccgg aggccaccaa ccggcgtgtc
  361 tgggaactgt ccaaggccaa ttcccgcttt gctaccactt tctatcagca cctggcagat
  421 tccaagaatg acaatgataa cattttcctg tcacccctga gtatctccac ggcttttgct
  481 atgaccaagc tgggtgcctg taatgacacc ctccagcaac tgatggaggt atttaagttt
  541 gacaccatat ctgagaaaac atctgatcag atccacttct tctttgccaa actgaactgc
  601 cgactctatc gaaaagccaa caaatcctcc aagttagtat cagccaatcg cctttttgga
  661 gacaaatccc ttaccttcaa tgagacctac caggacatca gtgagttggt atatggagcc
  721 aagctccagc ccctggactt caaggaaaat gcagagcaat ccagagcggc catcaacaaa
  781 tgggtgtcca ataagaccga aggccgaatc accgatgtca ttccctcgga agccatcaat
  841 gagctcactg ttctggtgct ggttaacacc atttacttca agggcctgtg gaagtcaaag
  901 ttcagccctg agaacacaag gaaggaactg ttctacaagg ctgatggaga gtcgtgttca
  961 gcatctatga tgtaccagga aggcaagttc cgttatcggc gcgtggctga aggcacccag
1021 gtgcttgagt tgcccttcaa aggtgatgac atcaccatgg tcctcatctt gcccaagcct
1081 gagaagagcc tggccaaggt ggagaaggaa ctcaccccag aggtgctgca ggagtggctg
1141 gatgaattgg aggagatgat gctggtggtt cacatgcccc gcttccgcat tgaggacggc
1201 ttcagtttga aggagcagct gcaagacatg ggccttgtcg atctgttcag ccctgaaaag
1261 tccaaactcc caggtattgt tgcagaaggc cgagatgacc tctatgtctc agatgcattc
1321 cataaggcat ttcttgaggt aaatgaagaa ggcagtgaag cagctgcaag taccgctgtt
1381 gtgattgctg gccgttcgct aaaccccaac agggtgactt tcaaggccaa caggcccttc
1441 ctggttttta taagagaagt tcctctgaac actattatct tcatgggcag agtagccaac
1501 ccttgtgtta agtaaaatgt tcttattctt tgcacctctt cctatttttg gtttgtgaac
1561 agaagtaaaa ataaatacaa actacttcca tctcacatt
```

What is claimed is:

1. A method of decreasing coagulation or thrombosis in a system, comprising administering an ATIII molecule to the system, wherein the ATIII molecule has an increased affinity for heparin or heparan sulfate proteoglycans bound to a solid surface, and wherein the ATIII binds the heparin or heparan sulfate proteoglycans under high wall shear rate conditions with a higher affinity than alpha ATIII.

2. The method of claim 1, further comprising a determination that high wall shear rates on the ATIII will occur.

3. The method of claim 1, wherein the system comprises heparin or heparan sulfate proteoglycans attached to a solid surface.

4. The method of claim 1, wherein the system comprises a graft.

5. The method of claim 1, wherein the system comprises a catheter.

6. The method of claim 1, wherein the system comprises a medical device.

7. The method of claim 1, wherein the system comprises stent, a heart pump, a heart lung bypass machine, a blood oxygenator, a ventricular assist device, an extracorporeal circuit, a blood gas sensor, an intraocular lens, or a heparin coated thermoplastic.

8. The method of claim 1, wherein the ATIII comprises a beta ATIII.

9. The method of claim 1, wherein the dissociation constant of ATIII for heparin is from 0.1 nM to 300 nM.

10. The method of claim 1, wherein the dissociation constant of ATIII for heparin is less than or equal to 54 nM.

11. The method of claim 1, wherein the dissociation constant of ATIII for heparin is less than or equal to 6 nM.

12. The method of claim 1, wherein the dissociation constant of ATIII for heparin is less than or equal to 1 nM.

13. The method of claim 1, wherein the affinity of the ATIII for heparin is at least 5 times that of alpha ATIII.

14. The method of claim 1, wherein the affinity of the ATIII for heparin is at least 50 times that of alpha ATIII.

15. The method of claim 1, wherein the affinity of the ATIII for heparin is at least 250 times that of alpha ATIII.

16. The method of claim 1, wherein the ATIII is produced in an insect or yeast expression system.

17. The method of claim 1, wherein the solid surface comprises a biomaterial.

18. The method of claim 1, wherein the shear rate conditions comprise shear rates of at least 1000 sec⁻¹.

19. The method of claim 1, wherein the wall shear rate conditions comprise shear rates of at least 1500 sec⁻¹.

20. The method of claim 1, wherein the wall shear rate conditions comprise shear rates of at least 2000 sec⁻¹.

21. The method of claim 1, wherein the wall shear rate conditions comprise shear rates of at least 2500 sec⁻¹.

22. The method of claim 1, wherein the wall shear rate conditions comprise shear rates of at least 3000 sec⁻¹.

23. A method of inhibiting coagulation under low and high wall shear rate conditions comprising administering an ATIII molecule, wherein the ATIII molecule binds heparin or heparan sulfate proteoglycans under low and high wall shear rate conditions with an affinity higher than alpha ATIII.

24. The method of claim 23, wherein the wherein the ATIII comprises a beta ATIII.

25. The method of claim 23, wherein the dissociation constant of ATIII for heparin is from 0.1 nM to 300 nM.

26. The method of claim 23, wherein the dissociation constant of ATIII for heparin is less than or equal to 54 nM.

27. The method of claim 23, wherein the dissociation constant of ATIII for heparin is less than or equal to 6 nM.

28. The method of claim 23, wherein the dissociation constant of ATIII for heparin is less than or equal to 1 nM.

29. The method of claim 23, wherein the affinity of the ATIII for heparin is at least 5 times that of alpha ATIII.

30. The method of claim 23, wherein the affinity of the ATIII for heparin is at least 50 times that of alpha ATIII.

31. The method of claim 23, wherein the affinity of the ATIII for heparin is at least 250 times that of alpha ATIII.

32. The method of claim 23, wherein the ATIII is produced in an insect or yeast expression system.

33. The method of claim 23, wherein the shear rate conditions comprise shear rates of at least 1000 sec⁻¹.

34. The method of claim 23, wherein the shear rate conditions comprise shear rates of at least 1500 sec⁻¹.

35. The method of claim 23, wherein the shear rate conditions comprise shear rates of at least 2000 sec¹.

36. The method of claim 23, wherein the shear rate conditions comprise shear rates of at least 2500 sec⁻¹.

37. The method of claim 23, wherein the shear rate conditions comprise shear rates of at least 3000 sec⁻¹.

38. A method of inhibiting coagulation or thrombosis during or following a cardiovascular procedure on a subject comprising administering high affinity ATIII molecules to the subject, wherein the ATIII molecules bind heparin or heparan sulfate proteoglycans under low and high wall shear rate conditions with an affinity higher than alpha ATIII.

39. The method of claim 38, wherein the ATIII is administered upstream of the area where ATIII loading is desired.

40. The method of claim 38, wherein the wherein the ATIII comprises a beta ATIII.

41. The method of claim 38, wherein the dissociation constant of ATIII for heparin is from 0.1 nM to 300 nM.

42. The method of claim 38, wherein the dissociation constant of ATIII for heparin is less than or equal to 54 nM.

43. The method of claim 38, wherein the dissociation constant of ATIII for heparin is less than or equal to 6 nM.

44. The method of claim 38, wherein the dissociation constant of ATIII for heparin is less than or equal to 1 nM.

45. The method of claim 38, wherein the affinity of the ATIII for heparin is at least 5 times that of alpha ATIII.

46. The method of claim 38, wherein the affinity of the ATIII for heparin is at least 50 times that of alpha ATIII.

47. The method of claim 38, wherein the affinity of the ATIII for heparin is at least 250 times that of alpha ATIII.

48. The method of claim 38, wherein the ATIII is produced in an insect or yeast expression system.

49. The method of claim 38, further comprising administering heparin, heparan sulfate proteoglycans, or systemic anticoagulants.

50. A method of preconditioning a heparin or heparan sulfate polyglycan coated material, comprising incubating the material with a solution comprising ATIII molecules, such that the ATIII molecules bind to the heparin or heparan sulfate proteoglycans under low and high wall shear rate conditions with an affinity higher than alpha ATIII.

51. The method of claim 50, wherein the wherein the ATIII comprises a beta ATIII.

52. The method of claim 50, wherein the dissociation constant of ATIII for heparin is from 0.1 nM to 300 nM.

53. The method of claim 50, wherein the dissociation constant of ATIII for heparin is less than or equal to 54 nM.

54. The method of claim 50, wherein the dissociation constant of ATIII for heparin is less than or equal to 6 nM.

55. The method of claim 50, wherein the dissociation constant of ATIII for heparin is less than or equal to 1 nM.

56. The method of claim 50, wherein the affinity of the ATIII for heparin is at least 5 times that of alpha ATIII.

57. The method of claim 50, wherein the affinity of the ATIII for heparin is at least 50 times that of alpha ATIII.

58. The method of claim 50, wherein the affinity of the ATIII for heparin is at least 250 times that of alpha ATIII.

59. The method of claim 50, wherein the ATIII is produced in an insect or yeast expression system.

60. A method of determining an amount of heparin or HSPG on a surface, the method comprising:
a. contacting the surface with a composition comprising an ATIII molecule at a wall shear rate, wherein the ATIII molecule has an increased affinity for heparin or heparan sulfate proteoglycans; and
b. assaying the amount of the ATIII molecule bound to the surface,
the amount of the ATIII bound to the surface being the minimum amount of heparin or heparan sulfate proteoglycan on the surface.

61. The method of claim 60, wherein the surface is contacted with an excess of the ATIII molecule, the amount of ATIII bound to the surface being the amount of heparin or heparan sulfate proteoglycan bound to the surface.

62. The method of claim 60, wherein the shear rate is 50, 630, 1500, or 3500 sec⁻¹.

63. The method of claim 60, wherein the wherein the ATIII comprises beta ATIII.

64. The method of claim 60, wherein the dissociation constant of ATIII for heparin is from 0.1 nM to 300 nM.

65. The method of claim 60, wherein the dissociation constant of ATIII for heparin is less than or equal to 54 nM.

66. The method of claim 60, wherein the dissociation constant of ATIII for heparin is less than or equal to 6 nM.

67. The method of claim 60, wherein the dissociation constant of ATIII for heparin is less than or equal to 1 nM.

68. The method of claim 60, wherein the affinity of the ATIII for heparin is at least 5 times that of alpha ATIII.

69. The method of claim 60, wherein the affinity of the ATIII for heparin is at least 50 times that of alpha ATIII.

70. The method of claim 60, wherein the affinity of the ATIII for heparin is at least 250 times that of alpha ATIII.

71. The method of claim 60, wherein the ATIII is produced in an insect or yeast expression system.

72. A method of determining a wall shear rate on a heparin or HSPG coated surface, the method comprising:
a. contacting the surface with a composition comprising an ATIII molecule, wherein the ATIII molecule has an increased affinity for heparin or heparan sulfate proteoglycan; and
b. assaying the amount of ATIII bound to the surface,
the higher the amount of ATIII bound to the surface the higher the wall shear rate.

73. A method of coating a surface with heparin or heparan sulfate proteoglycan, the method comprising:
a. determining an amount of ATIII that binds to the surface, where the ATIII has a high affinity for heparin or heparan sulfate proteoglycan; and
b. coating the surface with heparin or heparan sulfate proteoglycan in an amount at least that of ATIII bound to the surface.

74. A variant of ATIII, comprising a substitution at Y131 or its positional equivilant.

75. The variant of claim 74, wherein the substitution at Y131 or its positional equivilant is alanine, arginine, asparagine, aspartic acid, cysteine, glutamic acid, glutamine, glycine, histidine, isoleucine, lysine, leucine, methionine, serine, threonine, tryptophan, valine.

76. The variant of claim 74, wherein the substitution at Y131 or its positional equivilant is leucine.

77. The variant of claim 74, wherein the substitution at Y131 or its positional equivilant is alanine.

78. The variant of claim 74, wherein the variant's affinity for heparin is at least 5 times that of alpha ATIII.

79. The variant of claim 74, wherein the variant's affinity for heparin is at least 50 times that of alpha ATIII.

80. The variant of claim 74, wherein the variant's affinity for heparin is at least 250 times that of alpha ATIII.

81. The variant of claim 74, wherein the dissociation constant for heparin is less than or equal to 1 nM.

82. An ATIII variant, wherein the heparin binding affinity and basal factor Xa rate are increased by disrupting interactions between helix D and sheet A of the native ATIII.


SEQUENCE LISTING (2 sequences)

SEQ ID NO: 1 - protein, 464 aa, Artificial Sequence
  Description of Artificial Sequence/note = Synthetic Construct
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 2 - DNA, 1599 bp, Artificial Sequence
  Description of Artificial Sequence/note = Synthetic Construct
caccagcatcatctcctccaattcatccagctactctgcccatgaagataatagttttcaggcggattgcctcagatcacactatctccacttgcccagccctgtggaagattagcggccatgtattccaatgtgataggaactgtaacctctggaaaaaggaaggtttatcttttgtccttgctgctcattggcttctgggactgcgtgacctgtcacgggagccctgtggacatctgcacagccaagccgcgggacattcccatgaatcccatgtgcatttaccgctccccggagaagaaggcaactgaggatgagggctcagaacagaagatcccggaggccaccaaccggcgtgtctgggaactgtccaaggccaattcccgctttgctaccactttctatcagcacctggcagattccaagaatgacaatgataacattttcctgtcacccctgagtatctccacggcttttgctatgaccaagctgggtgcctgtaatgacaccctccagcaactgatggaggtatttaagtttgacaccatatctgagaaaacatctgatcagatccacttcttctttgccaaactgaactgccgactctatcgaaaagccaacaaatcctccaagttagtatcagccaatcgcctttttggagacaaatcccttaccttcaatgagacctaccaggacatcagtgagttggtatatggagccaagctccagcccctggacttcaaggaaaatgcagagcaatccagagcggccatcaacaaatgggtgtccaataagaccgaaggccgaatcaccgatgtcattccctcggaagccatcaatgagctcactgttctggtgctggttaacaccatttacttcaagggcctgtggaagtcaaagttcagccctgagaacacaaggaaggaactgttctacaaggctgatggagagtcgtgttcagcatctatgatgtaccaggaaggcaagttccgttatcggcgcgtggctgaaggcacccaggtgcttgagttgcccttcaaaggtgatgacatcaccatggtcctcatcttgcccaagcctgagaagagcctggccaaggtggagaaggaactcaccccagaggtgctgcaggagtggctggatgaattggaggagatgatgctggtggttcacatgccccgcttccgcattgaggacggcttcagtttgaaggagcagctgcaagacatgggccttgtcgatctgttcagccctgaaaagtccaaactcccaggtattgttgcagaaggccgagatgacctctatgtctcagatgcattccataaggcatttcttgaggtaaatgaagaaggcagtgaagcagctgcaagtaccgctgttgtgattgctggccgttcgctaaaccccaacagggtgactttcaaggccaacaggcccttcctggtttttataagagaagttcctctgaacactattatcttcatgggcagagtagccaacccttgtgttaagtaaaatgttcttattctttgcacctcttcctatttttggtttgtgaacagaagtaaaaataaatacaaactacttccatctcacatt